SEQUENCE LISTING (108 sequences)

SEQ ID NO: 1 - protein, 1272 aa, Artificial Sequence
  ABCA4-1150-CfaN-3FT
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPDYKDHDGDYKDHDIDYKDDDDK

SEQ ID NO: 2 - protein, 1182 aa, Artificial Sequence
  ABCA4-1150-CfaC-3FT
MVKIISRKSLGTQNVYDIGVEKDHNFLLKNGLVASNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 3 - protein, 1262 aa, Artificial Sequence
  ABCA4-1140-CfaN-3FT
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPDYKDHDGDYKDHDIDYKDDDDK

SEQ ID NO: 4 - protein, 1192 aa, Artificial Sequence
  ABCA4-1140-CfaCmut-3FT
MVKIISRKSLGTQNVYDIGVGEPHNFLLKNGLVASNCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 5 - protein, 1310 aa, Artificial Sequence
  ABCA4-1188-CfaN-3FT
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPDYKDHDGDYKDHDIDYKDDDDK

SEQ ID NO: 6 - protein, 1144 aa, Artificial Sequence
  ABCA4-1188-Cfacmut-3FT
MVKIISRKSLGTQNVYDIGVGEPHNFLLKNGLVASNCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 7 - protein, 2295 aa, Artificial Sequence
  ABCA4-3FT
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 8 - protein, 1273 aa, Artificial Sequence
  ABCA4-1150-NpuN-3FT
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNDYKDHDGDYKDHDIDYKDDDDK

SEQ ID NO: 9 - protein, 1182 aa, Artificial Sequence
  ABCA4-1150-NpuC-3FT
MIKIATRKYLGKQNVYDIGVERDHNFALKNGFIASNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 10 - protein, 1263 aa, Artificial Sequence
  ABCA4-1140-NpuN-3FT
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNDYKDHDGDYKDHDIDYKDDDDK

SEQ ID NO: 11 - protein, 1192 aa, Artificial Sequence
  ABCA4-1140-NpuC-3FT
MIKIATRKYLGKQNVYDIGVERDHNFALKNGFIASNCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 12 - protein, 1311 aa, Artificial Sequence
  ABCA4-1188-NpuN-3FT
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNDYKDHDGDYKDHDIDYKDDDDK

SEQ ID NO: 13 - protein, 1144 aa, Artificial Sequence
  ABCA4-1188-NpuC-3FT
MIKIATRKYLGKQNVYDIGVERDHNFALKNGFIASNCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 14 - protein, 179 aa, Artificial Sequence
  EGFP-71- CfaN
MVSKGEELFTGVVPILVELDGDVNGHKFSVSGEGEGDATYGKLTLKFICTTGKLPVPWPTLVTTLTYGVQCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPGHHHHHHG

SEQ ID NO: 15 - protein, 213 aa, Artificial Sequence
  EGFP-71- CfaC
MVKIISRKSLGTQNVYDIGVEKDHNFLLKNGLVASNCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGHHHHHHG

SEQ ID NO: 16 - protein, 247 aa, Artificial Sequence
  EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGHHHHHHG

SEQ ID NO: 17 - protein, 180 aa, Artificial Sequence
  EGFP-71- NpuN
MVSKGEELFTGVVPILVELDGDVNGHKFSVSGEGEGDATYGKLTLKFICTTGKLPVPWPTLVTTLTYGVQCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGHHHHHHG

SEQ ID NO: 18 - protein, 213 aa, Artificial Sequence
  EGFP-71- NpuC
MIKIATRKYLGKQNVYDIGVERDHNFALKNGFIASNCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGHHHHHHG

SEQ ID NO: 19 - protein, 1371 aa, Artificial Sequence
  ABCA4-1150-CfaN-3FT-SopE
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPDYKDHDGDYKDHDIDYKDDDDKTKITLSPQNFRIQKQETTLLKEKSTEKNSLAKSILAVKNHFIELRSKLSERFISHKNTESSATHFHRGSASEGRAVLTNKVVKDFMLQTLNDIDIRGSA

SEQ ID NO: 20 - protein, 1281 aa, Artificial Sequence
  ABCA4-1150-CfaC-3FT-SopE
MTKITLSPQNFRIQKQETTLLKEKSTEKNSLAKSILAVKNHFIELRSKLSERFISHKNTESSATHFHRGSASEGRAVLTNKVVKDFMLQTLNDIDIRGSAVKIISRKSLGTQNVYDIGVEKDHNFLLKNGLVASNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 21 - protein, 1361 aa, Artificial Sequence
  ABCA4-1140-CfaN-3FT-SopE
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPDYKDHDGDYKDHDIDYKDDDDKTKITLSPQNFRIQKQETTLLKEKSTEKNSLAKSILAVKNHFIELRSKLSERFISHKNTESSATHFHRGSASEGRAVLTNKVVKDFMLQTLNDIDIRGSA

SEQ ID NO: 22 - protein, 1291 aa, Artificial Sequence
  ABCA4-1140-CfaC-3FT-SopE
MTKITLSPQNFRIQKQETTLLKEKSTEKNSLAKSILAVKNHFIELRSKLSERFISHKNTESSATHFHRGSASEGRAVLTNKVVKDFMLQTLNDIDIRGSAVKIISRKSLGTQNVYDIGVGEPHNFLLKNGLVASNCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDDDK

SEQ ID NO: 23 - protein, 278 aa, Artificial Sequence
  EGFP-71-CfaN-SopE
MVSKGEELFTGVVPILVELDGDVNGHKFSVSGEGEGDATYGKLTLKFICTTGKLPVPWPTLVTTLTYGVQCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPGHHHHHHGTKITLSPQNFRIQKQETTLLKEKSTEKNSLAKSILAVKNHFIELRSKLSERFISHKNTESSATHFHRGSASEGRAVLTNKVVKDFMLQTLNDIDIRGSA

SEQ ID NO: 24 - protein, 312 aa, Artificial Sequence
  EGFP-71-CfaC-SopE
MTKITLSPQNFRIQKQETTLLKEKSTEKNSLAKSILAVKNHFIELRSKLSERFISHKNTESSATHFHRGSASEGRAVLTNKVVKDFMLQTLNDIDIRGSAVKIISRKSLGTQNVYDIGVEKDHNFLLKNGLVASNCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGHHHHHHG

SEQ ID NO: 25 - protein, 192 aa, Artificial Sequence
  EGFP-71-CfaN-DD1
MVSKGEELFTGVVPILVELDGDVNGHKFSVSGEGEGDATYGKLTLKFICTTGKLPVPWPTLVTTLTYGVQCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPGHHHHHHGRISFGSPPPMAGG

SEQ ID NO: 26 - protein, 226 aa, Artificial Sequence
  EGFP-71-CfaC-DD1
MRISFGSPPPMAGGVKIISRKSLGTQNVYDIGVEKDHNFLLKNGLVASNCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGHHHHHHG

SEQ ID NO: 27 - protein, 101 aa, Artificial Sequence
  CfaN
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 28 - protein, 36 aa, Artificial Sequence
  CfaC
MVKIISRKSLGTQNVYDIGVEKDHNFLLKNGLVASN

SEQ ID NO: 29 - protein, 36 aa, Artificial Sequence
  CfaCmut
MVKIISRKSLGTQNVYDIGVGEPHNFLLKNGLVASN

SEQ ID NO: 30 - protein, 30 aa, Artificial Sequence
  CatN
CLSGDTMIEILDDDGIIQKISMEDLYQRLA

SEQ ID NO: 31 - protein, 104 aa, Artificial Sequence
  CatC
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHN

SEQ ID NO: 32 - protein, 102 aa, Artificial Sequence
  NpuN
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 33 - protein, 36 aa, Artificial Sequence
  NpuC
MIKIATRKYLGKQNVYDIGVERDHNFALKNGFIASN

SEQ ID NO: 34 - protein, 28 aa, Artificial Sequence
  M3
KSVTLESRSPKFLNWFSVFSLFKVITTG

SEQ ID NO: 35 - protein, 21 aa, Artificial Sequence
  M4
YMSILRCASGKISIAAPPYIF

SEQ ID NO: 36 - protein, 36 aa, Artificial Sequence
  NpuCmut
MIKIATRKYLGKQNVYDIGVGEPHNFALKNGFIASN

SEQ ID NO: 37 - protein, 34 aa, Artificial Sequence
  M5
AGESFNFMVKLLYKHPILPCLKTLLSIRSSCSPR

SEQ ID NO: 38 - protein, 88 aa, Artificial Sequence
  GP41N
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 39 - protein, 102 aa, Artificial Sequence
  ConN
CLSYDTEILTVEYGAVPIGKIVEENIECTVYSVDENGFVYTQPIAQWHDRGEQEVFEYCLEDGSTIRATKDHKFMTEDGEMLPIDEIFEQGLDLKQVKGLPD

SEQ ID NO: 40 - protein, 16 aa, Artificial Sequence
  CL1
ACKNWFSSLSHFVIHL

SEQ ID NO: 41 - protein, 62 aa, Artificial Sequence
  Deg1
MNKIPIKDLLNPQITDEFKSSILDINKKLFSICCNLPKLPESVTTEEEVELRDILGFLSRAN

SEQ ID NO: 42 - protein, 30 aa, Artificial Sequence
  PEST
RSSSPSDSDTSGFSSGSDHLSDLISSLRIS

SEQ ID NO: 43 - protein, 13 aa, Artificial Sequence
  DD1
  VARIANT 12: X= any aminoacid, preferably Gly
RISFGSPPPMAXG

SEQ ID NO: 44 - protein, 9 aa, Artificial Sequence
  DD2
  VARIANT 8: X= any aminoacid, preferably Gly
GSPPPMAXG

SEQ ID NO: 45 - protein, 11 aa, Artificial Sequence
  DD3
TNGILKLGCQG

SEQ ID NO: 46 - protein, 17 aa, Artificial Sequence
  M1
CSEIIPMSRSTPISTMG

SEQ ID NO: 47 - protein, 19 aa, Artificial Sequence
  M2
VSFAFNLNSLIVGILRFHW

SEQ ID NO: 48 - protein, 100 aa, Artificial Sequence
  SopE
MTKITLSPQNFRIQKQETTLLKEKSTEKNSLAKSILAVKNHFIELRSKLSERFISHKNTESSATHFHRGSASEGRAVLTNKVVKDFMLQTLNDIDIRGSA

SEQ ID NO: 49 - protein, 78 aa, Artificial Sequence
  SopE-1-78
MTKITLSPQNFRIQKQETTLLKEKSTEKNSLAKSILAVKNHFIELRSKLSERFISHKNTESSATHFHRGSASEGRAVL

SEQ ID NO: 50 - protein, 63 aa, Artificial Sequence
  SopE-15-78
QETTLLKEKSTEKNSLAKSILAVKNHFIELRSKLSERFISHKNTESSATHFHRGSASEGRAVL

SEQ ID NO: 51 - protein, 35 aa, Artificial Sequence
  SopE-15-50
QETTLLKEKSTEKNSLAKSILAVKNHFIELRSKLS

SEQ ID NO: 52 - protein, 35 aa, Artificial Sequence
  L2
SLISLPLPTRVKFSSLLLIRIMKIITMTFPKKLRS

SEQ ID NO: 53 - protein, 16 aa, Artificial Sequence
  L6
FYYPIWFARVLLVHYQ

SEQ ID NO: 54 - protein, 46 aa, Artificial Sequence
  L9
SNPFSSLFGASLLIDSVSLKSNWDTSSSSCLISFFSSVMFSSTTRS

SEQ ID NO: 55 - protein, 39 aa, Artificial Sequence
  L10
CRQRFSCHLTASYPQSTVTPFLAFLRRDFFFLRHNSSAD

SEQ ID NO: 56 - protein, 46 aa, Artificial Sequence
  L11
GAPHVVLFDFELRITNPLSHIQSVSLQITLIFCSLPSLILSKFLQV

SEQ ID NO: 57 - protein, 39 aa, Artificial Sequence
  L12
NTPLFSKSFSTTCGVAKKTLLLAQISSLFFLLLSSNIAV

SEQ ID NO: 58 - protein, 45 aa, Artificial Sequence
  L15
PTVKNSPKIFCLSSSPYLAFNLEYLSLRIFSTLSKCSNTLLTSLS

SEQ ID NO: 59 - protein, 30 aa, Artificial Sequence
  L16
SNQLKRLWLWLLEVRSFDRTLRRPWIHLPS

SEQ ID NO: 60 - protein, 1149 aa, Artificial Sequence
  ABCA4N-1150
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKN

SEQ ID NO: 61 - protein, 1124 aa, Artificial Sequence
  ABCA4C-1150
CFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 62 - protein, 1139 aa, Artificial Sequence
  ABCA4N-1140
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLY

SEQ ID NO: 63 - protein, 1134 aa, Artificial Sequence
  ABCA4C-1140
CSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 64 - protein, 1187 aa, Artificial Sequence
  ABCA4N-1188
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTT

SEQ ID NO: 65 - protein, 1086 aa, Artificial Sequence
  ABCA4C-1188
CPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 66 - protein, 1250 aa, Artificial Sequence
  ABCA4-1150-CfaN
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 67 - protein, 1160 aa, Artificial Sequence
  ABCA4-1150-CfaC
MVKIISRKSLGTQNVYDIGVEKDHNFLLKNGLVASNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 68 - protein, 1240 aa, Artificial Sequence
  ABCA4-1140-CfaN
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 69 - protein, 1170 aa, Artificial Sequence
  ABCA4-1140-CfaCmut
MVKIISRKSLGTQNVYDIGVGEPHNFLLKNGLVASNCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 70 - protein, 1288 aa, Artificial Sequence
  ABCA4-1188-CfaN
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 71 - protein, 1122 aa, Artificial Sequence
  ABCA4-1188-Cfacmut
MVKIISRKSLGTQNVYDIGVGEPHNFLLKNGLVASNCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 72 - protein, 1349 aa, Artificial Sequence
  ABCA4-1150-CfaN-SopE
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTKITLSPQNFRIQKQETTLLKEKSTEKNSLAKSILAVKNHFIELRSKLSERFISHKNTESSATHFHRGSASEGRAVLTNKVVKDFMLQTLNDIDIRGSA

SEQ ID NO: 73 - protein, 1259 aa, Artificial Sequence
  ABCA4-1150-CfaC-SopE
MTKITLSPQNFRIQKQETTLLKEKSTEKNSLAKSILAVKNHFIELRSKLSERFISHKNTESSATHFHRGSASEGRAVLTNKVVKDFMLQTLNDIDIRGSAVKIISRKSLGTQNVYDIGVEKDHNFLLKNGLVASNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 74 - protein, 1339 aa, Artificial Sequence
  ABCA4-1140-CfaN-SopE
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPTKITLSPQNFRIQKQETTLLKEKSTEKNSLAKSILAVKNHFIELRSKLSERFISHKNTESSATHFHRGSASEGRAVLTNKVVKDFMLQTLNDIDIRGSA

SEQ ID NO: 75 - protein, 1269 aa, Artificial Sequence
  ABCA4-1140-CfaCmut-SopE
MTKITLSPQNFRIQKQETTLLKEKSTEKNSLAKSILAVKNHFIELRSKLSERFISHKNTESSATHFHRGSASEGRAVLTNKVVKDFMLQTLNDIDIRGSAVKIISRKSLGTQNVYDIGVGEPHNFLLKNGLVASNCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 76 - protein, 1263 aa, Artificial Sequence
  ABCA4-1150-CfaN-DD1
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPRISFGSPPPMAGG

SEQ ID NO: 77 - protein, 1173 aa, Artificial Sequence
  ABCA4-1150-CfaC-DD1
MRISFGSPPPMAGGVKIISRKSLGTQNVYDIGVEKDHNFLLKNGLVASNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 78 - protein, 1253 aa, Artificial Sequence
  ABCA4-1140-CfaN-DD1
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPRISFGSPPPMAGG

SEQ ID NO: 79 - protein, 1183 aa, Artificial Sequence
  ABCA4-1140-CfaCmut-DD1
MRISFGSPPPMAGGVKIISRKSLGTQNVYDIGVGEPHNFLLKNGLVASNCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 80 - protein, 13 aa, Artificial Sequence
  DD4
  VARIANT 12: X= any aminoacid, preferably Gly
RDSFGSPPPMAXG

SEQ ID NO: 81 - protein, 4 aa, Artificial Sequence
  DD5
GFWI

SEQ ID NO: 82 - protein, 4 aa, Artificial Sequence
  DD6
RFKG

SEQ ID NO: 83 - protein, 4 aa, Artificial Sequence
  DD7
KFYK

SEQ ID NO: 84 - protein, 8 aa, Artificial Sequence
  Hexahistidine
GHHHHHHG

SEQ ID NO: 85 - protein, 23 aa, Artificial Sequence
  V12
TNGSVLREFTLLELEVVTRNTEL

SEQ ID NO: 86 - protein, 21 aa, Artificial Sequence
  T1
SVLEENRPFAQQLSNVYFTIL

SEQ ID NO: 87 - protein, 1176 aa, Artificial Sequence
  ABCA4N-1177
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGT

SEQ ID NO: 88 - protein, 1097 aa, Artificial Sequence
  ABCA4C-1177
CSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 89 - protein, 1178 aa, Artificial Sequence
  ABCA4N-1179
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCS

SEQ ID NO: 90 - protein, 1095 aa, Artificial Sequence
  ABCA4C-1179
CSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 91 - protein, 1277 aa, Artificial Sequence
  ABCA4-1177-CfaN
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 92 - protein, 1133 aa, Artificial Sequence
  ABCA4-1177-CfaCmut
MVKIISRKSLGTQNVYDIGVGEPHNFLLKNGLVASNCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 93 - protein, 1279 aa, Artificial Sequence
  ABCA4-1179-CfaN
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 94 - protein, 1131 aa, Artificial Sequence
  ABCA4-1179-CfaCmut
MVKIISRKSLGTQNVYDIGVGEPHNFLLKNGLVASNCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 95 - protein, 1095 aa, Artificial Sequence
  ABCA4N-1096
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPY

SEQ ID NO: 96 - protein, 1178 aa, Artificial Sequence
  ABCA4C-1096
SRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 97 - protein, 1184 aa, Artificial Sequence
  ABCA4N-1185
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGF

SEQ ID NO: 98 - protein, 1089 aa, Artificial Sequence
  ABCA4C-1185
STTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 99 - protein, 1183 aa, Artificial Sequence
  ABCA4-1096-gp41N
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 100 - protein, 1215 aa, Artificial Sequence
  ABCA4-1096-gp41C
MMLKKILKIEELDERELIDIEVSGNHLFYANDILTHNSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 101 - protein, 1272 aa, Artificial Sequence
  ABCA4-1185-gp41N
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 102 - protein, 1126 aa, Artificial Sequence
  ABCA4-1185-gp41C
MMLKKILKIEELDERELIDIEVSGNHLFYANDILTHNSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 103 - protein, 22 aa, Artificial Sequence
  3FT
DYKDHDGDYKDHDIDYKDDDDK

SEQ ID NO: 104 - protein, 37 aa, Artificial Sequence
  GP41C
MMLKKILKIEELDERELIDIEVSGNHLFYANDILTHN

SEQ ID NO: 105 - protein, 36 aa, Artificial Sequence
  ConC
MVKIISRQSLGKQNVYDIGVEKDHNFLLANGLIASN

SEQ ID NO: 106 - protein, 2273 aa, Artificial Sequence [sequence omitted]

SEQ ID NO: 107 - protein, 21 aa, Artificial Sequence
  T2
KLKGLGKRCKRREDLEIRFIL

SEQ ID NO: 108 - protein, 21 aa, Artificial Sequence
  T3
WKLLLWVGLVLVLKHHDGAAH

(19) United States
(12) Patent Application Publication
(10) Pub. No.: US 2023/0116688 A1
(43) Pub. Date: Apr. 13, 2023

(54) SPLIT INTEINS AND THEIR USES

(71) Applicant: SPLICEBIO, S.L., Barcelona (ES)

(72) Inventors: Silvia FRUTOS DOMÍNGUEZ, Barcelona (ES); Gerard CAELLES VIDAL, Barcelona (ES); Anabel OTERO BILBAO, Barcelona (ES); Miquel VILA PERELLÓ, Barcelona (ES)

(21) Appl. No.: 17/914,212

(22) PCT Filed: Mar. 26, 2021
(86) PCT No.: PCT/EP2021/058016

§ 371 Date: Sep. 23, 2022

(30) Foreign Application Priority Data
Mar. 26, 2020 (EP) 20382236.6

(51) Int. Cl. C07K 14/47; C12N 15/86

(52) U.S. Cl. CPC C07K 14/47; C12N 15/86; C07K 2319/60; C07K 2319/92; C12N 2750/14143

(57) ABSTRACT

The present invention relates to methods of use of engineered split inteins, as well as their combination with degradation signals (destabilizing domains, degrons) to reconstitute large genes for gene therapy.

### TECHNICAL FIELD OF THE INVENTION

The present invention is comprised within the field of biotechnology, it specifically relates to split inteins and their uses.

### BACKGROUND OF THE INVENTION

It is known that genes that cannot be encapsulated inside an AAV (adeno-associated virus) for gene therapy, can be split in two fragments. Each fragment should be thus fused to a split intein and delivered in their own AAV. Upon infection of the target cells inteins will enable the generation of the desired target protein (see FIG. 1).

Nevertheless, one of the key limitations of the approach is the accumulation of protein intermediates (N-Extein-ItnN and IntC-C-Extein), as well as the excised split intein. To address this problem, in the instant application, we have shown that certain inteins can be combined with certain degradation signals providing a significant reduction in the accumulation of such protein intermediates. The instant invention focuses on this combination of inteins and degradation signals. We have also identified certain intein-degron combinations that contribute to increase splicing yields in vivo.

### BRIEF DESCRIPTION OF THE INVENTION

In a preferred initial aspect, the present invention refers to a composition comprising:
a. a first polynucleotide encoding a polypeptide comprising a Split intein N-fragment, wherein the Split intein N-fragment is selected from the list consisting of the CfaN of SEQ ID NO 27, the CatN of SEQ ID NO 30 and the Gp41N of SEQ ID NO 38, or any functionally equivalent variants thereof such as ConN of SEQ ID NO 39, directly linked via a peptide bond, optionally through a peptide linker, to the N-terminal fragment of a protein to be reconstituted; and
b. a second polynucleotide encoding a polypeptide comprising a Split intein C-fragment, wherein the Split intein C-fragment is selected from the list consisting of the CfaC of SEQ ID NO 28, the CatC of SEQ ID NO 31 and the Gp41C of SEQ ID NO 104, or any functionally equivalent variants thereof such as CfaCmut (SEQ ID NO 29) or ConC (SEQ ID NO 105), directly linked via a peptide bond, optionally through a peptide linker, to the C-terminal fragment of the protein to be reconstituted;
wherein both polynucleotides of the composition may be packed together in a single formulation or separately in different formulations;
wherein the first and the second polynucleotides, respectively, encode the N-terminal fragment and the C-terminal fragment of the protein to be reconstituted, in such a way that when both fragments are combined, the N-terminal fragment of the protein is linked to the C-terminal fragment of the protein generating the whole protein;
wherein the protein to be reconstituted is of more than 25 KDa; and
wherein the composition is further characterized in that:
the split intein N-fragment is further directly linked via a peptide bond to a degron, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and/or
the split intein C-fragment is further directly linked via a peptide bond to a degron, wherein the degron is linked to the intein C-fragment via the N-terminus of the intein, with or without a linker between the intein C-fragment and the degron, and wherein the C-terminus of the Split intein C-fragment is directly linked via a peptide bond to the C-terminal fragment of the protein to be reconstituted

In a preferred embodiment of the initial aspect, the degron directly linked to the split intein N-fragment and/or split intein C-fragment is selected from the list consisting of CL1, Deg1, PEST, DD1, DD2, DD3, M1, M2, SopE, SopE-1-78, SopE-15-78, SopE-15-50, L2, L6, L9, L10, L11, L12, L1S, L16, M3, M4, M5, V12, DD4, DD5, DD6 and DD7. Preferably, the degron directly linked to the split intein N-fragment and/or split intein C-fragment is selected from the list consisting of DD1, DD3, PEST, SopE, L2, L9, M4, or V12. More preferably, the degron directly linked to the split intein N-fragment and/or split intein C-fragment is selected from the list consisting of SopE, L2, L9, M4, or V12.

In another preferred embodiment of the initial aspect, the Split intein N-fragment is the CfaN of SEQ ID NO 27, directly linked via a peptide bond, optionally through a peptide linker, to the N-terminal fragment of a protein to be reconstituted, and the Split intein C-fragment is the CfaC of SEQ ID NO 28, directly linked via a peptide bond, optionally through a peptide linker, to the C-terminal fragment of the protein to be reconstituted; wherein, preferably, the degron directly linked to the split intein N-fragment and/or split intein C-fragment is selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7; more preferably, the degron directly linked to the split intein N-fragment and/or split intein C-fragment is selected from the list consisting of DD1, DD3, PEST, SopE, L2, L9, M4, or V12; still more preferably, the degron directly linked to the split intein N-fragment and/or split intein C-fragment is selected from the list consisting of SopE, L2, L9, M4, or V12.

In another preferred embodiment of the initial aspect, the Split intein N-fragment is the CfaN of SEQ ID NO 27, directly linked via a peptide bond, optionally through a peptide linker, to the N-terminal fragment of a protein to be reconstituted, and the Split intein C-fragment is the CfaCmut of SEQ ID NO 29, directly linked via a peptide bond, optionally through a peptide linker, to the C-terminal fragment of the protein to be reconstituted; wherein, preferably, the degron directly linked to the split intein N-fragment and/or split intein C-fragment is selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7; more preferably, the degron directly linked to the split intein N-fragment and/or split intein C-fragment is selected from the list consisting of DD1, DD3, PEST, SopE, L2, L9, M4, or V12; still more preferably, the degron directly linked to the split intein N-fragment and/or split intein C-fragment is selected from the list consisting of SopE, L2, L9, M4, or V12.

In another preferred embodiment of the initial aspect, the Split intein N-fragment is the NpuN of SEQ ID NO 32, directly linked via a peptide bond, optionally through a peptide linker, to the N-terminal fragment of a protein to be reconstituted, and the Split intein C-fragment is the CfaCmut of SEQ ID NO 29, or the NpuCmut SEQ ID NO 36, directly linked via a peptide bond, optionally through a peptide linker, to the C-terminal fragment of the protein to be reconstituted; wherein, preferably, the degron directly linked to the split intein N-fragment and/or split intein C-fragment is selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7; more preferably, the degron directly linked to the split intein N-fragment and/or split intein C-fragment is selected from the list consisting of DD1, DD3, PEST, SopE, L2, L9, M4, or V12; still more preferably, the degron directly linked to the split intein N-fragment and/or split intein C-fragment is selected from the list consisting of SopE, L2, L9, M4, or V12.

In another preferred embodiment of the initial aspect, the composition is characterized by comprising two degrons, in particular, the composition is characterized in that:
the split intein N-fragment is further directly linked via a peptide bond to a degron, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
the split intein C-fragment is further directly linked via a peptide bond to a degron, wherein the degron is linked to the intein C-fragment via the N-terminus of the intein, with or without a linker between the intein C-fragment and the degron, and wherein the C-terminus of the Split intein C-fragment is directly linked via a peptide bond to the C-terminal fragment of the protein to be reconstituted

In a preferred embodiment of the initial aspect or of any of its preferred embodiments, the composition is characterized in that it comprises:
a. the first polynucleotide encoding a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and optionally further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encoding a polypeptide comprising a Split intein C-fragment selected from the list consisting of CfaC of SEQ ID NO 28 or CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and optionally further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted.

In a preferred embodiment of the initial aspect or of any of its preferred embodiments, the composition comprises:
a. the first polynucleotide encoding a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and optionally further directly linked via a peptide bond to a degron selected from the list consisting of DD1, DD3, PEST, SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encoding a polypeptide comprising a Split intein C-fragment selected from the list consisting of CfaC of SEQ ID NO 28 or CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and optionally further directly linked via a peptide bond to a degron selected from the list consisting of DD1, DD3, PEST, SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted.

In another preferred embodiment of the initial aspect or of any of its preferred embodiments, the composition comprises:
a. the first polynucleotide encoding a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and optionally further directly linked via a peptide bond to a degron selected from the list consisting of SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encoding a polypeptide comprising a Split intein C-fragment selected from the list consisting of CfaC of SEQ ID NO 28 or CfaCmut of SEQ ID NO 29, or any functionally equivalent variant thereof, directly linked via a peptide bond, optionally through a peptide linker, to the C-terminal fragment of a protein to be reconstituted, and optionally further directly linked via a peptide bond to a degron selected from the list consisting of SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted.

In another preferred embodiment of the initial aspect or of any of its preferred embodiments, the composition comprises:
a. the first polynucleotide encoding a polypeptide comprising a Split intein N-fragment selected from the list consisting of Gp41N of SEQ ID NO 38 or any functionally equivalent variant thereof, directly linked via a peptide bond, optionally through a peptide linker, to the N-terminal fragment of a protein to be reconstituted, and optionally further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encoding a polypeptide comprising a Split intein C-fragment selected from the list consisting of Gp41C of SEQ ID NO 104 or any functionally equivalent variant thereof, directly linked via a peptide bond, optionally through a peptide linker, to the C-terminal fragment of a protein to be reconstituted, and optionally further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted.

In another preferred embodiment of the initial aspect or of any of its preferred embodiments, the first and the second polynucleotides encode the N-terminal fragment and the C-terminal fragment of the ABCA4 protein respectively, in such a way that when both polynucleotides are translated into their respective protein complexes and combined according to the methods of the invention, the N-terminal fragment of the ABCA4 protein is linked to the C-terminal fragment of the ABCA4 protein thus generating the whole ABCA4 protein.

In connection to the above indicated embodiment wherein the first and the second polynucleotides encode the N-terminal fragment and the C-terminal fragment of the ABCA4 protein respectively, the present invention provides the following alternatives.

First alternative:
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and optionally further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and optionally further directly linked via a peptide bond to a degron selected from the list consisting of CL1, Deg1, PESt, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
   wherein said first polynucleotide encodes positions 1-1149, 1-1139, 1-1176, or 1-1178 of the N-terminal fragment of the ABCA4 protein; and wherein the second polynucleotide encodes positions 1150-2273, 1140-2273, 1177-2273, or 1179-2273 of the C-terminal fragment of the ABCA4 protein, wherein when said first polynucleotide encodes position 1-1149 the second polynucleotide encodes positions 1150-2273, wherein when said first polynucleotide encodes position 1-1139 the second polynucleotide encodes positions 1140-2273, wherein when said first polynucleotide encodes position 1-1176 the second polynucleotide encodes positions 1177-2273, and wherein when said first polynucleotide encodes position 1-1178 the second polynucleotide encodes positions 1179-2273.

Second alternative:
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and optionally further directly linked via a peptide bond to a degron selected from the list consisting of DD1, DD3, PEST, SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and optionally further directly linked via a peptide bond to a degron selected from the list consisting of DD1, DD3, PEST, SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
   wherein said first polynucleotide encodes positions 1-1149, 1-1139, 1-1176, or 1-1178 of the N-terminal fragment of the ABCA4 protein; and wherein the second polynucleotide encodes positions 1150-2273, 1140-2273, 1177-2273, or 1179-2273 of the C-terminal fragment of the ABCA4 protein, wherein when said first polynucleotide encodes position 1-1149 the second polynucleotide encodes positions 1150-2273, wherein when said first polynucleotide encodes position 1-1139 the second polynucleotide encodes positions 1140-2273, wherein when said first polynucleotide encodes position 1-1176 the second polynucleotide encodes positions 1177-2273, and wherein when said first polynucleotide encodes position 1-1178 the second polynucleotide encodes positions 1179-2273.

Third alternative:
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and optionally further directly linked via a peptide bond to a degron selected from the list consisting of SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and optionally further directly linked via a peptide bond to a degron selected from the list consisting of SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
   wherein said first polynucleotide encodes positions 1-1149, 1-1139, 1-1176, or 1-1178 of the N-terminal fragment of the ABCA4 protein; and wherein the second polynucleotide encodes positions 1150-2273, 1140-2273, 1177-2273, or 1179-2273 of the C-terminal fragment of the ABCA4 protein, wherein when said first polynucleotide encodes position 1-1149 the second polynucleotide encodes positions 1150-2273, wherein when said first polynucleotide encodes position 1-1139 the second polynucleotide encodes positions 1140-2273, wherein when said first polynucleotide encodes position 1-1176 the second polynucleotide encodes positions 1177-2273, and wherein when said first polynucleotide encodes position 1-1178 the second polynucleotide encodes positions 1179-2273.

Fourth alternative:
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and optionally further directly linked via a peptide bond to a degron selected from the list consisting of DD1, DD3, PEST, SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaC of SEQ ID NO 28, CfaCmut of SEQ ID NO 29 or NpuCmut of SEQ ID NO 36, or any functionally equivalent variants thereof, directly linked via a peptide bond, optionally through a peptide linker, to the C-terminal fragment of a protein to be reconstituted, and optionally further directly linked via a peptide bond to a degron selected from the list consisting of DD1, DD3, PEST, SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
   wherein said first polynucleotide encodes positions 1-1149 of the N-terminal fragment of the ABCA4 protein; and wherein the second polynucleotide encodes positions 1150-2273 of the C-terminal fragment of the ABCA4 protein, wherein when said first polynucleotide encodes position 1-1149 the second polynucleotide encodes positions 1150-2273.

Fifth alternative:
a. the first polynucleotide encodes a polypeptide comprising a Split intein N-fragment selected from the list consisting of Gp41N of SEQ ID NO 38 or any functionally equivalent variant thereof, directly linked via a peptide bond, optionally through a peptide linker, to the N-terminal fragment of a protein to be reconstituted, and further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of Gp41C of SEQ ID NO 104 or any functionally equivalent variant thereof, directly linked via a peptide bond, optionally through a peptide linker, to the C-terminal fragment of a protein to be reconstituted, and further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
   wherein said first polynucleotide encodes positions 1-1095, or 1-1185 of the N-terminal fragment of the ABCA4 protein; and wherein the second polynucleotide encodes positions 1096-2273, or 1186-2273 of the C-terminal fragment of the ABCA4 protein, wherein when said first polynucleotide encodes position 1-1095 the second polynucleotide encodes positions 1096-2273, and wherein when said first polynucleotide encodes position 1-1185 the second polynucleotide encodes positions 1186-2273.

In a further preferred embodiment of the initial aspect or of any of its preferred embodiments or alternatives, both polynucleotides are comprised within vectors that allow the propagation or insertion of said polynucleotides in suitable host cells. Preferably, said vectors are adeno associated viruses (AAV), more preferably the vectors are AAVs of serotype 1, 2, 3, 4, 5, 6, 7, 8, or 9.

In another further preferred embodiment of the initial aspect or of any of its preferred embodiments or alternatives, the compositions described herein are for use in therapy, in particular for any of the diseases identified in table 1.

A further aspect of the invention refers to an in vitro or in vivo method for expressing a gene of interest in a cell, which comprises:
(i) contacting the cell with
a first polynucleotide and a second polynucleotide as defined in any of the previous aspects, embodiments or alternatives, where preferably both polynucleotides are comprised within adeno associated viruses (AAV), and wherein at least one of the said polynucleotides encodes a split intein fragment directly linked via a peptide bond to a degron,
(ii) allowing the expression of the first and the second polynucleotides so that the first and the second fusion proteins are produced and
(iii) allowing the contact between the first and second proteins so that the split intein N-fragment binds to the split intein C-fragment to form an intein intermediate and the intein intermediate reacts to covalently link the C-terminus of the first polypeptide of interest to the N-terminus of the second polypeptide of interest.

### BRIEF DESCRIPTION OF THE FIGURES

FIG. 1. General scheme of the strategies to use inteins, and inteins combined with degrons to reconstitute large proteins for gene therapy. Top panel: (1) the gene of interest is recombinantly split in a judiciously selected position and the resulting 5′ end is recombinantly fused to and IntN, and the 3′ to the IntC, in such a way that upon protein expression the N-terminal fragment of the protein will be expressed with the IntN fused to its C-terminus, and the C-terminal fragment of the protein will be expressed with the IntC fused to its N-terminus. (2) Each of the construct is encapsulated in a separate AAV. These two AAVs are administered to a patient, so that they are co-transduced to certain cells (3). Upon co-transduction the DNA delivered via the AAVs will be transcribed into RNA and translated into proteins, and at the protein level the inteins will perform the protein trans-splicing reaction to reconstitute the desired spliced product (4). Bottom plane1: inteins are combined with degradation signals to prevent accumulation of the starting materials and eliminate the excised inteins. Degrons are fused to the C-terminus of the N-intein and/or the N-terminus of the C-intein.

FIG. 2. Top panel: reaction scheme between the EGFP^{N}-IntN-H6 and IntC-EGFP^{C}-H6 constructs, which results in the formation of full length EGFP with a H6 tag, and excision of the inteins. Lower panel: flourescence microscopy images of cells transfected with the N-, C-terminal fragments or co-transfected with both fragments, using either the Cfa or the Npu intein.

FIG. 3. Western blot analysis of lysed HEK293 cells transfected with EGFP-intein polynucleotides. Left panel: western blot analysis of cells transfected with EGFP-intein plasmids. Cells were transfected with a full length EGFP plasmid, or either a EGFP^{N}-IntN, an IntC-EGFP^{C} plasmid, or co-transfected with both. Plasmids containing the Cfa or the Npu intein were tested. Right panel: quantification of fold increase of spliced product, relative to Npu. Product yield was determined by densitometry, using β-tubulin as a loading control. The graph represents the fold increase of EGFP product, relative to Npu, when cells were transfected with either full length EGFP plasmids (EGFP), co-transfected with EGFP^{N}-CfaN and CfaC-EGFP^{C} (Cfa), or with EGFP^{N}-NpuN and NpuC-EGFP^{C} (Npu).

FIG. 4. HEK293 transfected cells were analyzed by flow cytometry. Left panel: flow cytometry data of cells transfected with different constructs or combinations of constructs. Black curves correspond to controls transfected with either an N-terminal fragment or C-terminal fragment of EGFP. In blue are 4 replicates of cells transfected with a plasmid encoding full length EGFP, green curves correspond to cells co-transfected with EGFP^{N}-CfaN and CfaC-EGFP^{C} (EGFP split at position 71), yellow curves correspond to cells co-transfected with EGFP^{N}-NpuN and NpuC-EGFP^{C} (EGFP split at position 71). Middle panel: Plot of Mean Fluorescence Intensity (MFI) obtained for each set of samples indicating the use of the Cfa intein allows to recover over 90% of the signal corresponding to full length EGFP. Right panel: plot representing the number of positive cells in each of the samples showing transfection efficiency was comparable for all three sets.

FIG. 5: Comparison of splicing yields of ABCA4 at position 1150 between Cfa and Npu. Cells were co-transfected with ABCA4^{N}1150-IntN and IntC-ABCA4^{C}1150, lysed and analyzed by WB. ABCA4^{N}1150 corresponds to the N-terminal fragment of ABCA4 ranging from residue 1 to 1149 and ABCA4^{C}1150 corresponds to the C-terminal fragment of ABCA4 ranging from residue 1150 to 2273. Constructs with either Cfa or Npu were tested. Left panel shows blot using an anti-ABCA4 mAb. Right panel corresponds to the densitometric quantification of western blots.

FIG. 6. Comparison of splicing yields of ABCA4 at position 1140 between Cfa and Npu. Cells were co-transfected with ABCA4^{N}1140-IntN and IntC-ABCA4^{C}1140, lysed and analyzed by WB. ABCA4^{N}1140 corresponds to the N-terminal fragment of ABCA4 ranging from residue 1 to 1139 and ABCA4^{C}1140 corresponds to the C-terminal fragment of ABCA4 ranging from residue 1140 to 2273. Constructs with either Cfa or Npu were tested. Left panel and middle panel show results blotted with an anti-FLAG tag and anti-ABCA4 mAb, respectively. Right panel corresponds to the densitometric quantification of the western blots.

FIG. 7: Comparison of splicing yields of ABCA4 at positions 1140, 1150, 1179 and 1188. Cells were co-transfected with the corresponding ABCA4^{N}-CfaN and CfaC (or CfaCmut)-ABCA4^{C} constructs, lysed and analyzed by WB. Graph corresponds to the densitometric quantification by WB of the reconstituted ABCA4 protein and its comparison of ABCA4 levels obtained upon transfection with a plasmid encoding for the full-length protein.

FIG. 8: Reaction scheme of PTS including degrons. The N-terminal fragment of the protein of interest is fused to the IntN, a linker, which in this case is a His6 tag, and a degron. The C-terminus of the protein is fused to the IntC, which in its N-terminus is linked to a degron. Splicing reaction results in the formation of the desired full-length protein, without any degron fused to it, and the cleaved inteins, each of them bearing a degradation signal.

FIG. 9: Combination of inteins and degrons to reconstitute reporter protein EGFP. HEK293 cells were transfected with the indicated constructs, lysed and analyzed by western blot using an anti-His6 tag mAb. Results indicate that the use of constructs combining CfaN or C inteins with a degradation signal resulted in disappearance of any signal arising from starting materials (EGFP-CfaN-6H) as well as from the intein (CfaN-6H).

FIG. 10: Combination of inteins and degrons to reconstitute large protein ABCA4. HEK293 cells were transfected with ABCA4^{N}1150-CfaN and CfaC-ABCA4^{C}1150, with and without degrons, lysed and analyzed by western blot using an anti-FLAG tag mAb. Cells at two different timepoints were taken (24 and 48 h). Top panel: western blot analysis. Bottom panel: densitometric quantification of western blot. Results indicate that the use of constructs combining Cfa N or C inteins with a degradation signal resulted in disappearance of signal arising from starting materials (ABCA4-CfaN and CfaC-ABCA4). Interestingly, results also demonstrate that the combination of the Cfa intein with a degradation signal results in an increase in the yields of reconstituted full length ABCA4.

FIG. 11: Effect of intein-degron combination is mediated by the proteosome. Cells were co-transfected with ABCA4^{N}1150-CfaN and CfaC-ABCA4^{C}1150 constructs, with and without degrons; after 24 h cells were incubated for the indicated amount of time (30 min, 3, 6 and 24 h) in the presence, or absence, of the proteosome inhibitor MG132. After the indicated amount of time cells were lysed and analyzed by western blot using an anti-FLAG mAb. The addition of the proteosome inhibitor MG132 causes an increase of the intensity of the bands corresponding to the fragments, suggesting their degradation has been inhibited.

FIG. 12: Comparison of reconstitution yields using different inteins or inteins combined with degrons. Left panel: Western blot of reconstitution via PTS of ABCA4 split at position 1150 using Cfa, Cfa-SopE or Npu. Cells were co-transfected with ABCA4^{N}1150-CfaN and CfaC-ABCA4^{C}1150 (Cfa), ABCA4^{N}1150-CfaN-SopE and SopE-CfaC-ABCA4^{C}1150 (Cfa-SopE), or ABCA4^{N}1150-NpuN and NpuC-ABCA4^{C}1150 (Npu). After 48 h cells were collected, lysed and analysed by WB. A duplicate for the Cfa constructs and a triplicate for the CfaSopE and Npu were analyzed. Right panel: Densitometry quantification of the product obtained with each combination of inteins and degrons, as well as remaining starting materials. Results show how reconstitution of product is higher when using Cfa combined with a degron (SopE). Interestingly, data shows that reconstitution with Npu is the lowest, but also that it is the one with the highest level of unreacted starting materials left over.

FIG. 13: Reconstitution of ABCA4 at position 1140 using CfaCmut, with and without degradation signal. Left panel: Cells were co-transfected with ABCA4^{N}1140-CfaN and CfaCmut-ABCA4^{C}1140, ABCA4^{N}1140-CfaN-SopE and SopE-CfaC-ABCA4^{C}1140 (Cfa-SopE), or full length ABCA4 (ABCA). After 48 h cells were collected, lysed and analyzed by WB using an anti-FLAG tag mAb. Right panel: densitometric quantification of level of ABCA4 reconstitution, compared to transfection with full length ABCA4.

FIG. 14: Combination of inteins and degrons to reconstitute reporter protein EGFP, using DD1 degron. HEK293 cells were transfected with the indicated constructs, lysed and analyzed by western blot using an anti-His6 tag mAb. Combination if the Cfa inteins with DD1 degron results in elimination of undesired starting materials and excised intein.

FIG. 15: Mass spectrometry analysis. Cells were transfected with ABCA4^{N}1150-CfaN-SopE and SopE-CfaC-ABCA4^{C}1150, and after 48 h were collected and lysed. Cell lysate was run in an SDSPAGE gel and a band around 300 KDa was cut, proteolyzed (see materials and methods) and analyzed by LC-MS/MS. Results are summarized in the figure. In green are shown peptides identified by MSMS with a False Discovery Rate (FDR) below 1%. In yellow peptides identified with FDR below 5%. Underlined in red is shown the sequence of the split site.

FIG. 16: Three-piece ligation using orthogonal consensus inteins.

FIG. 17: Three-piece ligation using orthogonal consensus inteins. The N, M and C constructs shown in FIG. 16 were transfected in HEK293 either alone, co-transfected in pairs, or the three fragments co-transfected together. Cells were lysed and analyzed by Western blot using an antibody against a 3FT tag present at the C-terminus of the N, M- and C-terminal fragments (left panel) or an antibody against the POIN-fragment (right).

FIG. 18: A) Analysis of the effect of degrons on the expression levels of split intein N-fragments of the invention. Constructs with and without degrons were transfected into HEK293 cells, and expression levels analyzed by Western blot. Addition of the degron to the N-intein fragment results in a reduction of the detected levels of expressed protein. B) Analysis of the effect of degrons on the expression levels of split intein C-fragments of the invention. Constructs with and without degrons were transfected into HEK293 cells, and expression levels analyzed by Western blot. Addition of the degron to the C-intein fragment results in a reduction of the detected levels of expressed protein. C) Analysis by Western blot of cells transfected with either the N- or C-terminal fragment, or co-transfected with both fragments are shown. Results of constructs without degron (no degron), or with representative degrons (SopE, L2 and L9) are shown. The right-hand panel provides the quantification of the amount of PTS product obtained with each of the indicated degrons, compared to those obtained without degron. It can be seen that some degrons provide higher yield than the constructs without degron. Interestingly, all degrons work, and with all of them at least 25% of the product obtained without degron can be reconstituted. Some degrons allow to reconstitute more than 50% of the levels obtained without degron, and finally, there are some degrons that yield 100% (or higher) reconstitution.

FIG. 19. The PTS reactions with different degrons were analyzed by WB using a different type of gel to be able to detect the inteins. As it can be seen several degrons reduce the amount of excised inteins compared to others (for example SpoE and PEST), while others, like L2, L9, M2, M4 or DD3, completely eliminate the intein band).

FIG. 20. Analysis by Western blot of the PTS reaction at the split position 1150 with different degron combinations and without degron. In this case the degron at the N-fragment was fixed (left panel L9 and right panel L2, labeled with an N) and different degrons were used at the C-fragment (L2, L9 M4, M2, V12, DD3 and DD1, labeled with a C).

FIG. 21: WB and quantification of PTS reactions at site 1140 and 11 1179 with or without the degron L9 in the N- and C-terminal intein ABCA4 fragment.

FIG. 22: Analysis by Western blot of the PTS reaction using the gp41 intein. The Left panel: Western blot of reconstitution via PTS of ABCA4 split at position 1150 using Cfa intein and position 1185 using gp41 intein. After 48 h cells were collected, lysed and analyzed by WB. Right panel: Densitometry quantification of the product obtained with each inteins, as well as the full-length protein.

FIG. 23: in vivo retina EGFP: (A) Fundus Auto Fluorescence (FAF) of mice injected with AAV8s encoding full-length EGFP, or two AAVs encoding for the N (N, EGFP^{N}-CfaN) and C-terminal fragments of the invention (C, CfaC-EGFP^{C}). (B) Immuno histochemical analysis (IHC) of saline or treated retinas. Results indicate native EGFP fluorescence in photo-receptors, indicative of protein splicing mediated EGFP reconstitution. All constructs were under the control of the GRK1 promoter, and two different doses of the N+C constructs were injected. BSS (Balanced Sterile Saline).

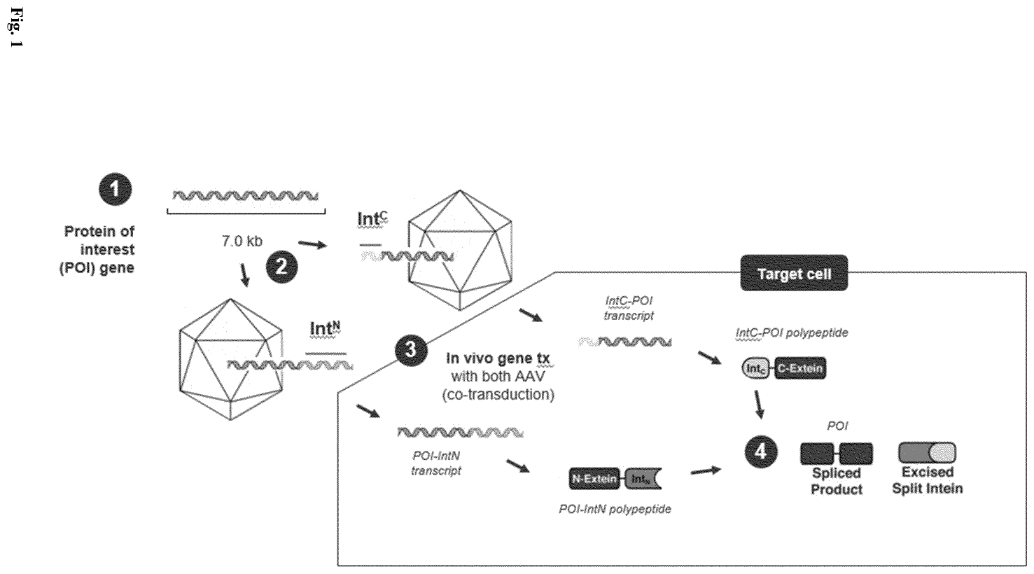

### DESCRIPTION OF THE INVENTION

The present invention relates to methods of use of engineered or naturally occurring split inteins, as well as their combination with degradation signals (destabilizing domains, degrons) to reconstitute large proteins for gene therapy.

The methods described in the present invention allow to reconstitute large proteins in vitro, ex vivo and in vivo. When used in vivo it would allow to reconstitute desired large target proteins in any desired tissue including, but not limited to, the central nervous system (CNS), peripheral nervous system (PNS), muscle, liver, eye, pancreas, retina, kidney, inner ear, heart, lung, blood, spleen, skin.

As used herein, the term “intein” means a naturally-occurring or artificially-constructed polypeptide sequence capable of catalyzing a protein splicing reaction that excises the intein sequence from a precursor protein and joins the flanking sequences (N- and C-exteins) with a peptide bond. They are typically 150-550 amino acids in size and may also contain a homing endonuclease domain. A list of known inteins is published at http://www.inteins.com and https://inteins.biocenter.helsinkifi/index.php

The term “split intein” as used herein refers to any intein in which the N-terminal and C-terminal amino acid sequences are not directly linked via a peptide bond, such that the N-terminal and C-terminal sequences become separate fragments that can non-covalently re-associate, or reconstitute, into an intein that is functional for trans-splicing reactions.

The term “peptide bond” refers to a covalent chemical bond —CO—NH— formed between two molecules when the carboxy part of one molecule, referred to as a carboxy component, reacts with the amino part of another molecule, referred to as an amino component, causing the release of a molecule. For example, proteinogenic L-amino acids can form the peptide bond upon joining with the release of a molecule of water. Therefore, proteins and peptides can be regarded as chains of amino acid residues held together by peptide bonds. A peptide bond is an “amide bond” or “amide linkage”.

The terms “polypeptide”, “peptide” or “protein” are used interchangeably herein to refer to polymers of amino acids.

The term “amino acid” refers to naturally occurring and synthetic amino acids, as well as amino acid analogs and amino acid mimetics that function in a manner similar to the naturally occurring amino acids. Furthermore, the term “amino acid” includes both D- and L-amino acids (stereoisomers).

The term “natural amino acids” or “naturally occurring amino acid” comprises the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine.

As used herein the term “non-natural amino acid” or “synthetic amino acid” refers to a carboxylic acid, or a derivative thereof, substituted at position “a” (alpha) with an amine group and being structurally related to a natural amino acid. Illustrative non-limiting examples of modified or uncommon amino acids include 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxy lysine, alio hydroxy lysine, 3-hydroxyproline, 4-hydroxyproline, isodesmosine, alloisoleucine, N-methylglycine, N-methyliso leucine, 6-N-methyl-lysine, N-methylvaline, norvaline, norleucine, ornithine, p-acetylphenylalanina, p-halophenylalanina, p-proparglyoxyphenylalanina, p-azidophenylalanina, p-benzoylphenylalanina, etc. This group also includes the D-isomers of the “natural amino acids”.

As used herein, the term “split intein N-fragment” or “N-terminal split intein” or “N-terminal intein fragment” or “N-terminal intein sequence” (abbreviated “IntN”) refers to any intein sequence that comprises an N-terminal amino acid sequence that is functional for trans-splicing reactions, that is, that is capable of associating with a functional split intein C-fragment to form a complete intein that is capable of excising itself from the host protein, catalyzing the ligation of the extein or flanking sequences with a peptide bond, or that upon association with a split intein C-fragment catalyzes the “N-terminal cleavage”, that is, the nucleophilic attack of the peptide bond between the extein and the N-terminus of the split intein N-fragment resulting in the breaking of said peptide bond. An lntN thus also comprises a sequence that is spliced out when trans-splicing occurs. An lntN can comprise a sequence that is a modification of the N-terminal portion of a naturally occurring intein sequence. For example, it can comprise additional amino acid residues and/or mutated residues so long as the inclusion of such additional and/or mutated residues does not render the lntN non-functional in trans-splicing. Preferably, the inclusion of the additional and/or mutated residues improves or enhances the trans-splicing activity of the lntN.

As used herein, the term “Degrons” means a naturally-occurring or artificially-constructed polypeptide sequence which when recombinantly fused to another polypeptide it accelerates its protein degradation via the proteosomal degradation pathway, or any other cellular degradation mechanism.

As interchangeably used herein, the terms “split intein C-fragment”, “C-terminal split intein”, “C-terminal intein fragment” and “C-terminal intein sequence” (abbreviated “lntC”) refer to any intein sequence that comprises a C-terminal amino acid sequence that is functional for trans-splicing reactions, that is, that upon association is capable of associating with a functional split intein N-fragment to form a complete intein that is capable of excising itself from the host protein, catalyzing the ligation of the extein or flanking sequences with a peptide bond, or that upon association with a split N-intein catalyzes the “C-terminal cleavage”, that is, the nucleophilic attack of the peptide bond between the extein and the C-terminus of the split intein C-fragment resulting in the breaking of said peptide bond. An lntC thus also comprises a sequence that is spliced out when trans-splicing occurs. An lntC can comprise a sequence that is a modification of the C-terminal portion of a naturally occurring intein sequence. For example, it can comprise additional amino acid residues and/or mutated residues so long as the inclusion of such additional and/or mutated residues does not render the lntC non-functional in trans-splicing. Preferably, the inclusion of the additional and/or mutated residues improves or enhances the trans-splicing activity of the lntC.

It is herein noted that, in the context of the present invention, “the N-terminal fragment of a protein to be reconstituted” refers to the N-terminal fragment of a protein, more particularly, a fragment of a protein of more than 25 KDa, more than 50 KDa or more than 100 KDa. The term “N-terminal fragment of a protein”, as used herein, thus refers to a fragment of variable length that includes the N-terminus of the protein (in its mature or immature form). In a particular embodiment, the N-terminal fragment is a fragment comprising less than 100%, less than 90%, less than 80%, less than 70%, less than 60%, less than 50%, less than 40%, less than 30%, less than 20%, less than 10%, less than 5% of the length of the whole protein.

It is herein noted that, in the context of the present invention, “the C-terminal fragment of a protein to be reconstituted” refers to the C-terminal fragment of a protein, more particularly, a fragment of a protein of more than 25 KDa, more than 50 KDa or more than 100 KDa. The term “C-terminal fragment of a protein”, as used herein, thus refers to a fragment of variable length that includes the C-terminus of the protein. In a particular embodiment, the C-terminal fragment is a fragment comprising less than 100%, less than 90%, less than 80%, less than 70%, less than 60%, less than 50%, less than 40%, less than 30%, less than 20%, less than 10%, less than 5% of the length of the whole protein.

As used herein, the term “polynucleotide” refers to a polymer composed of a multiplicity of nucleotide units (deoxyribonucleotides or ribonucleotides, or related structural variants or synthetic analogues thereof) linked via phosphodiester bonds (or related structural variants on synthetic analogues thereof). The term polynucleotide includes double or single stranded genomic and cDNA, RNA, any synthetic and genetically manipulated polynucleotide, and both sense and anti-sense polynucleotide (although only sense stands are being disclosed in the present invention). This includes single- and double-stranded molecules, i.e., DNA-DNA, DNA-RNA and RNA-RNA hybrids.

Split Intein N-Fragments

The present invention refers to a split intein N-fragment directly linked via a peptide bond to the N-terminal fragment of a protein to be reconstituted. The construct would have the general architecture from the N to the C-terminus:
(N-terminal fragment of the protein to be reconstituted)-InteinN.

The protein to be reconstituted can be any large gene, whose reconstitution could result in a positive therapeutic effect. A non-limited example of proteins that could be reconstituted with the instant invention, is the ABCA4 protein, and the proteins encoded by the genes listed in the following table 1. The goal of reconstituting these proteins is to treat diseases associated with mutations in their encoding genes, by providing correct versions of such genes. Additionally, the protein to be reconstituted could be a protein or enzyme to treat a disease, but not necessarily to replace a mutated one. For example, the protein to be reconstituted could be CRISPR/Cas9 systems for gene, base or prime editing.

TABLE 1

| Disease | Protein (gene) | Uniprot ID | Coding region Size |
|---|---|---|---|
| Usher Syndrome 2C | Adhesion G-protein coupled | Q8WXG9 | 18.9 |
| (USH2C) | receptor V1 (ADGRV1) | | |
| Macular degeneration, | Hemicentin-1 (HMCN1) | Q96RW7 | 18.9 |
| age-lreated, 1 (ARMD1) | | | |
| Febrile seizures, familial, | Adhesion G-protein coupled | Q8WXG9 | 18.9 |
| 4 (FEB4) | receptor V1 (ADGRV1) | | |
| Usher Syndrome 2A | Usherin (USH2A) | O75445 | 15.6 |
| (USH2A) | | | |
| Retinitis pigmentosa 39 | Usherin (USH2A) | O75445 | 15.6 |
| (RP39) | | | |
| Alstom Syndrome | Alstrom syndrome protein 1 | Q8TCU4 | 12.5 |
| | (ALMS1) | | |
| Duchenne Muscular | Dystrophin (DMD) | P11532 | 11.6 |
| dystrophy | | | |
| Deafness, autosomal | Unconventional myosin-XV | Q9UKN7 | 10.8 |
| recessive, 3 (DFNB3) | (MYO15A) | | |
| Usher syndrome 1D/F | Cadherin-23 (CDH23) | Q9H251 | 10.1 |
| (USH1DF) | | | |
| Usher syndrome 1D | Cadherin-23 (CDH23) | Q9H251 | 10.1 |
| (USH1D) | | | |
| Pituitary adenoma 5, | Cadherin-23 (CDH23) | Q9H251 | 10.1 |
| multiple types (PITA5) | | | |
| Deafness, autosomal | Cadherin-23 (CDH23) | Q9H251 | 10.1 |
| recessive, 12 (DFNB12) | | | |
| Retinitis pigmentosa 25 | Protein eyes shut homolog (EYS) | Q5T1H1 | 9.4 |
| (RP25) | | | |
| Muscular dystrophy, | Laminin subunit alpha-2 | P24043 | 9.4 |
| limb-girdle ar 23 | (LAMA2) | | |
| (LGMDR23) | | | |
| Merosin-deficient | Laminin subunit alpha-2 | P24043 | 9.4 |
| congenital muscular | (LAMA2) | | |
| dystrophy 1A (MDC1A) | | | |
| Von Willebrand disease 1 | von Willebrand factor (VWF) | P04275 | 8.4 |
| (VWD1) | | | |
| Von Willebrand disease 2 | von Willebrand factor (VWF) | P04275 | 8.4 |
| (VWD2) | | | |
| Von Willebrand disease 3 | von Willebrand factor (VWF) | P04275 | 8.4 |
| (VWD3) | | | |
| Transient bullous dermolysis | Collagen alpha-1(VII) chain | Q02388 | 8.1 |
| of the newborn (TBDN) | (COL7A1) | | |
| Nail disorder, no-syndromic | Collagen alpha-1(VII) chain | Q02388 | 8.1 |
| congenital, 8 (NDNC8) | (COL7A1) | | |
| Epidermolysis bullosa | Collagen alpha-1(VII) chain | Q02388 | 8.1 |
| dystrophica, with | (COL7A1) | | |
| subcorneal cleavage | | | |
| (EBDSC) | | | |
| Epidermolysis bullosa | Collagen alpha-1(VII) chain | Q02388 | 8.1 |
| dystrophica, Bart type (B-DEB) | (COL7A1) | | |
| Epidemolysis bullosa | Collagen alpha-1(VII) chain | Q02388 | 8.1 |
| pruriginosa (EBP) | (COL7A1) | | |
| Epidemolysis bullosa | Collagen alpha-1(VII) chain | Q02388 | 8.1 |
| dystrophica, pretibial | (COL7A1) | | |
| type (PR-DEB) | | | |
| Epidemolysis bullosa | Collagen alpha-1(VII) chain | Q02388 | 8.1 |
| dystrophica ar (RDEB) | (COL7A1) | | |
| Epidemolysis bullosa | Collagen alpha-1(VII) chain | Q02388 | 8.1 |
| dystrophica ad (DDEB) | (COL7A1) | | |
| Spinocerebellar ataxia 6 | Voltage-dependent P/Q-type | O00555 | 7.7 |
| (SCA6) | calcium channel subunit alpha-1A | | |
| | (CACNA1A) | | |
| Migraine, familial | Voltage-dependent P/Q-type | O00555 | 7.7 |
| hemiplegic, 1 (FHM1) | calcium channel subunit alpha-1A | | |
| | (CACNA1A) | | |
| Episodic ataxia 2 (EA2) | Voltage-dependent P/Q-type | O00555 | 7.7 |
| | calcium channel subunit alpha-1A | | |
| | (CACNA1A) | | |
| Epileptic encephalopathy, | Voltage-dependent P/Q-type | O00555 | 7.7 |
| early infantile, 42 | calcium channel subunit alpha-1A | | |
| (EIEE42) | (CACNA1A) | | |
| Senior-Loken syndrome 6 | Centrosomal protein of 290 kDa | O15078 | 7.5 |
| (SLSN6) | (CEP290) | | |
| Meckel syndrome 4 | Centrosomal protein of 290 kDa | O15078 | 7.5 |
| (MKS4) | (CEP290) | | |
| Leber Congenital | Centrosomal protein of 290 kDa | O15078 | 7.5 |
| Amaurosis 10 (LCA10) | (CEP290) | | |
| Joubert syndrome 5 | Centrosomal protein of 290 kDa | O15078 | 7.5 |
| (JBTS5) | (CEP290) | | |
| Bardet-Biedl syndrome | Centrosomal protein of 290 kDa | O15078 | 7.5 |
| 14 (BBS14) | (CEP290) | | |
| Occult macular dystrophy | Retinitis pigmentosa 1-like 1 | Q8IWN7 | 7.2 |
| (OCMD) | protein (RP1L1) | | |
| Hyperaldosteronism, | Voltage-dependent T-type | O95180 | 7.1 |
| familial, 4 (HALD4) | calcium channel subunit alpha- | | |
| | 1H (CACNA1H) | | |
| Hemophilia A | Coagulation factor FVIII | P00451 | 7.1 |
| Epilepsy, idiopathic | Voltage-dependent T-type | O95180 | 7.1 |
| generalized 6 (EIG6) | calcium channel subunit alpha- | | |
| | 1H (CACNA1H) | | |
| Epilepsy, childhood | Voltage-dependent T-type | O95180 | 7.1 |
| absence 6 (ECA6) | calcium channel subunit alpha- | | |
| | 1H (CACNA1H) | | |
| Deafness, ar, 28 | TRIO and F-actin-binding protein | Q9H2D6 | 7.1 |
| (DFNB28) | (TRIOBP) | | |
| Retinitis pigmentosa 13 | Pre-mRNA-processing-splicing | Q6P2Q9 | 7.0 |
| (RP13) | factor 8 (PRPF8) | | |
| Stargardt disease 1 | Retinal-specific phospholipid- | P78363 | 6.8 |
| (STGD1) | transporting ATPase ABCA4 (ABCA4) | | |
| Retinitis pigmentosa 19 | Retinal-specific phospholipid- | P78363 | 6.8 |
| (RP19=) | transporting ATPase ABCA4 (ABCA4) | | |
| Macular degeneration, | Retinal-specific phospholipid- | P78363 | 6.8 |
| age-related, 2 (ARMD2) | transporting ATPase ABCA4 (ABCA4) | | |
| Fundus flavimaculatus | Retinal-specific phospholipid- | P78363 | 6.8 |
| (FFM) | transporting ATPase ABCA4 (ABCA4) | | |
| Cone-rod dystrophy 3 | Retinal-specific phospholipid- | P78363 | 6.8 |
| (CORD3) | transporting ATPase ABCA4 (ABCA4) | | |
| Usher syndrome 1B (USH1B) | Unconventional myosin-VIIa (MYO7A) | Q13402 | 6.7 |
| Deafness ar 2 (DFNB2) | Unconventional myosin-VIIa (MYO7A) | Q13402 | 6.7 |
| Deafness ad 11 (DFNA11) | Unconventional myosin-VIIa (MYO7A) | Q13402 | 6.7 |
| Retinitis pigmentosa 1 (RP1) | Oxygen-regulated protein 1 (RP1) | P56715 | 6.5 |
| Muscular dystrophy, | Dysferlin (DYSF) | O75923 | 6.5 |
| limb-girdle, ar 2 | | | |
| (LGMDR2) | | | |
| Miyoshi muscular | Dysferlin (DYSF) | O75923 | 6.5 |
| dystrophy 1 (MMD1) | | | |
| Distal myopathy with | Dysferlin (DYSF) | O75923 | 6.5 |
| anterior tibial onset | | | |
| (DMAT) | | | |
| Deafness, ad, 12 (DFNA12) | Alpha-tectorin (TECTA) | O75443 | 6.5 |
| Deafness, ar, 21 (DFNB21) | Alpha-tectorin (TECTA) | O75443 | 6.5 |
| Retinitis pigmentosa 33 | U5 small nuclear | O75643 | 6.4 |
| (RP33) | ribonucleoprotein 200 kDa | | |
| | helicase (SNRNP200) | | |
| Progressive familial heart | Sodium channel protein type 5 | Q14524 | 6.2 |
| block 1A (PFHB1A) | subunit alpha (SCN5A) | | |
| Long QT syndrome 3 | Sodium channel protein type 5 | Q14524 | 6.2 |
| (LQT3) | subunit alpha (SCN5A) | | |
| Brugada syndrome 1 | Sodium channel protein type 5 | Q14524 | 6.2 |
| (BRGDA1) | subunit alpha (SCN5A) | | |
| Sick sinus syndrome 1 | Sodium channel protein type 5 | Q14524 | 6.2 |
| (SSS1) | subunit alpha (SCN5A) | | |
| Familial paroxysmal | Sodium channel protein type 5 | Q14524 | 6.2 |
| ventricular fibrillation 1 | subunit alpha (SCN5A) | | |
| (VF1) | | | |
| Sudden infant death | Sodium channel protein type 5 | Q14524 | 6.2 |
| syndrome (SIDS) | subunit alpha (SCN5A) | | |
| Atrial standstill 1 | Sodium channel protein type 5 | Q14524 | 6.2 |
| (ATRST1) | subunit alpha (SCN5A) | | |
| Cardiomyopathy, dilated | Sodium channel protein type 5 | Q14524 | 6.2 |
| 1E (CMD1E) | subunit alpha (SCN5A) | | |
| Atrial fibrillation, familial, | Sodium channel protein type 5 | Q14524 | 6.2 |
| 10 (ATFB10) | subunit alpha (SCN5A) | | |
| Seizures, benign familial | Sodium channel protein type 2 | Q99250 | 6.2 |
| infantile, 3 (BFIS3) | subunit alpha (SCN2A) | | |
| Migraine, familial | Sodium channel protein type 1 | P35498 | 6.2 |
| hemiplegic, 3 (FHM3) | subunit alpha (SCN1A) | | |
| Intractable childhood | Sodium channel protein type 1 | P35498 | 6.2 |
| epilepsy with generalized | subunit alpha (SCN1A) | | |
| tonic-clonic seizures (ICEGTC) | | | |
| Infantile spasms | Sodium channel protein type 2 | Q99250 | 6.2 |
| | subunit alpha (SCN2A) | | |
| Generalized epilepsy with | Sodium channel protein type 1 | P35498 | 6.2 |
| febrile seizures plus 2 | subunit alpha (SCN1A) | | |
| (GEFS + 2) | | | |
| Febrile seizures, familial, | Sodium channel protein type 1 | P35498 | 6.2 |
| 3A (FEB3A) | subunit alpha (SCN1A) | | |
| Epileptic enfephalopathy, | Sodium channel protein type 1 | P35498 | 6.2 |
| early infantile, 6 (EIEE6) | subunit alpha (SCN1A) | | |
| Epileptic encephalopathy, | Sodium channel protein type 3 | Q9NY46 | 6.2 |
| early infantile, 62 (EIEE62) | subunit alpha (SCN3A) | | |
| Epileptic encephalopathy, | Sodium channel protein type 2 | Q99250 | 6.2 |
| early infantile, 11 (EIEE11) | subunit alpha (SCN2A) | | |
| Epilepsy, familial focal, | Sodium channel protein type 3 | Q9NY46 | 6.2 |
| with variable foci 4 (FFEVF4) | subunit alpha (SCN3A) | | |
| Dravet Syndrome | Sodium channel protein type 1 | P35498 | 6.2 |
| | subunit alpha (SCN1A) | | |
| Deafness, ar, 9 (DFNB9) | Otoferlin (OTOF) | Q9HC10 | 6.2 |
| Auditory neuropathy, ar, | Otoferlin (OTOF) | Q9HC10 | 6.2 |
| 1 (AUNB1) | | | |
| Deafness, ad, 4A (DFNA4A) | Myosin-14 (MYH14) | Q7Z406 | 6.2 |
| Peripheral neuropathy, | Myosin-14 (MYH14) | Q7Z406 | 6.2 |
| myopathy, hoarseness | | | |
| and hearing liss (PNMHH) | | | |
| Deafness, ar, 77 | Lipoxygenase homology domain- | Q8IVV2 | 6.2 |
| (DFNB77) | containing protein 1 (LOXHD1) | | |
| Seizures, benign familial | Sodium channel protein type 8 | Q9UQD0 | 6.1 |
| infantile, 5 (BFIS5) | subunit alpha (SCN8A) | | |
| Myoclonus, familial, 2 | Sodium channel protein type 8 | Q9UQD0 | 6.1 |
| (MYOCL2) | subunit alpha (SCN8A) | | |
| Epileptic encephalopathy, | Sodium channel protein type 8 | Q9UQD0 | 6.1 |
| early infantile, 13 (EIEE13) | subunit alpha (SCN8A) | | |
| Cognitive impairment with or | Sodium channel protein type 8 | Q9UQD0 | 6.1 |
| without cerebellar ataxia (CIAT) | subunit alpha (SCN8A) | | |
| Night blindness, congenital | Voltage-dependent L-type calcium | O60840 | 6.0 |
| stationary 2A (CSNB2A) | channel subunit alpha-1F (CACNA1F) | | |
| Cone-rod dystrophy, X- | Voltage-dependent L-type calcium | O60840 | 6.0 |
| linked 3 (CORDX3) | channel subunit alpha-1F (CACNA1F) | | |
| Aaland island eye disease | Voltage-dependent L-type calcium | O60840 | 6.0 |
| (AIED) | channel subunit alpha-1F (CACNA1F) | | |
| Usher syndrome 1F (USH1F) | Photocadherin-15 (PCDH15) | Q96QU1 | 5.9 |
| Usher syndrome 1D/F (USH1DF) | Photocadherin-15 (PCDH15) | Q96QU1 | 5.9 |
| Deafness, ar, 23 (DFNB23) | Photocadherin-15 (PCDH15) | Q96QU1 | 5.9 |
| Deafness, ar, 16 (DFNB16) | Stereocilin (STRC) | Q7RTU9 | 5.2 |
| Deafness-infertility | Stereocilin (STRC) | Q7RTU9 | 5.2 |
| syndrome (DIS) | | | |
| Deafness, ar, 30 (DFNB30) | Myosin-IIIa (MYO3A) | Q8NEV4 | 4.8 |
| Otospondylomegaepiphyseal | Collagen alpha-2(XI) chain | P13942 | 4.7 |
| dysplasia ad (OSMEDA) | (COL11A2) | | |
| Otospondylomegaepiphyseal | Collagen alpha-2(XI) chain | P13942 | 4.7 |
| dysplasia ar (OSMEDB) | (COL11A2) | | |
| Deafness ad 13 (DFNA13) | Collagen alpha-2(XI) chain | P13942 | 4.7 |
| | (COL11A2) | | |
| Deafness ar 53 (DFNB53) | Collagen alpha-2(XI) chain | P13942 | 4.7 |
| | (COL11A2) | | |
| Fibrochondrogenesis 2 | Collagen alpha-2(XI) chain | P13942 | 4.7 |
| (FBCG2) | (COL11A2) | | |
| Cystic Fibrosis (CF) | Cystic fibrosis transmembrane | P13569 | 4.6 |
| | conductance regulator (CFTR) | | |
| Congenital bilateral | Cystic fibrosis transmembrane | P13569 | 4.6 |
| absence of the vas | conductance regulator (CFTR) | | |
| deferens (CBAVD) | | | |
| Fanconi anemia, | Fanconi anemia group A protein | O15360 | 4.4 |
| complementation group A | (FANCA) | | |
| (FANCA) | | | |
| Retinitis pigmentosa 12 | Protein crumbs homolog 1 | P82279 | 4.2 |
| (RP12) | (CRB1) | | |
| Pigmented paravenous | Protein crumbs homolog 1 | P82279 | 4.2 |
| chorioretinal atrophy | (CRB1) | | |
| (PPCRA) | | | |
| Leber congenital | Protein crumbs homolog 1 | P82279 | 4.2 |
| amaurosis 8 (LCA8) | (CRB1) | | |
| Deafness ad 22 (DFNA22) | Unconventional myosin-VI | Q9UM54 | 4.1 |
| | (MYO6) | | |
| Deafness ar 37 (DFNB37) | Unconventional myosin-VI | Q9UM54 | 4.1 |
| | (MYO6) | | |
| Deafness ad 22, with | Unconventional myosin-VI | Q9UM54 | 4.1 |
| hypertrophic | (MYO6) | | |
| cardiomyopathy (DFNHCM) | | | |
| Deafness, ar, 30 (DFNB30) | Myosin-IIIb (MYO3B) | Q8WXR4 | 4.0 |
| Leber congenital | X-linked retinitis pigmentosa | Q96KN7 | 4.0 |
| amaurosis 6 (LCA6) | GTPase regulator-interacting | | |
| | protein 1 (RPGRIP1) | | |
| Cone-rod dystrophy 13 | X-linked retinitis pigmentosa | Q96KN7 | 4.0 |
| (CORD13) | GTPase regulator-interacting | | |
| | protein 1 (RPGRIP1) | | |

LCA = leber congenital amaurosis
RP = retinitis pigmentosa
ad = autosomal dominant
ar = autosomal recessive
PR = photoreceptors
RPE = retinal pigment epithelium
ECM = extracellular matrix

Therefore, a first aspect the invention refers to a Split intein N-fragment directly linked via a peptide bond to the N-terminal fragment of a protein to be reconstituted, wherein, as reflected above, the IntN is linked, either directly or by using a linker, to the C-terminus of the N terminal fragment (from hereinafter “the split intein N-fragment of the invention”).

A preferred embodiment of the first aspect of the invention refers to a split intein N-fragment directly linked via a peptide bond to a degron (from hereinafter “the split intein N-fragment degron of the invention”), wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of a protein to be reconstituted.

Therefore, the architecture of the construct of the split intein N-fragment degron of the invention, from the N to the C-terminus would be:

(N-terminal portion of the protein to be reconstituted)-(Intein-N fragment)-(Degron).

If a linker is introduced between the IntN and the degron then the architecture of the construct of the split intein N-fragment degron of the invention, from the N to the C-terminus would be:

(N-terminal portion of the protein to be reconstituted)-(Intein-N fragment)-(linker)-(Degron)

Preferably, the Intein N of any of the split intein N-fragment of the invention or the split intein N-fragment degron of the invention can be selected from any of the following listed in table 2 below:

TABLE 2

| | | SEQ ID |
|---|---|---|
| Name | Sequence | NO |
| Cfa^{N} | CLSYDTEILTVEYGFLPIGKIVEERIECTVYTVDKN | 27 |
| | GFVYTQPIAQWHNRGEQEVFEYCLEDGSIIRATKDH | |
| | KFMTTDGQMLPIDEIFERGLDLKQVDGLP | |
| Cat^{N} | CLSGDTMIEILDDDGIIQKISMEDLYQRLA | 30 |
| Npu^{N} | CLSYETEILTVEYGLLPIGKIVEKRIECTVYSVDNN | 32 |
| | GNIYTQPVAQWHDRGEQEVFEYCLEDGSLIRATKDH | |
| | KFMTVDGQMLPIDEIFERELDLMRVDNLPN | |
| Gp41N | CLDLKTQVQTPQGMKEISNIQVGDLVLSNTGYNEVL | 38 |
| | NVFPKSKKKSYKITLEDGKEIICSEEHLFPTQTGEM | |
| | NISGGLKEGMCLYVKE | |
| ConN | CLSYDTEILTVEYGAVPIGKIVEENIECTVYSVDEN | 39 |
| | GFVYTQPIAQWHDRGEQEVFEYCLEDGSTIRATKDH | |
| | KFMTEDGEMLPIDEIFEQGLDLKQVKGLPD | |

wherein preferably the Intein N is the CfaN intein of SEQ ID NO 27 or any variants thereof such as ConN of SEQ ID NO 39; or the CatN intein of SEQ ID NO 30 or any variants thereof; or the NpuN intein of SEQ ID NO 32 or any variants thereof; or the Gp41^{N} intein of SEQ ID NO 38 or any variants thereof.

The term “variant” as used herein refers to a polypeptide molecule that is substantially similar to a particular polypeptide sequence. In the present invention we refer to variants of the N-inteins or the C-inteins of Cfa, Npu, Cat or Gp41 inteins, as polypeptides substantially similar to any of their polypeptide sequences, for example the Con intein is understood herein as a variant of Cfa. Thus, the variant may be similar in structure and biological activity to the polypeptide from which it derives. Therefore, the variant may refer to a mutant of a polypeptide sequence. The term “mutant” refers to a polypeptide molecule the sequence of which has one or more amino acids added, deleted, substituted or otherwise chemically modified in comparison to the polypeptide molecule from which it derives. The mutant may retain substantially the same properties as the polypeptide molecule from which it derives or lack the biological activity of the claimed sequences. In a particular embodiment, intein variants include mutants in which the non-catalytic Cys residues, that is the 1st residue of the N-intein, have been mutated to Serine, or Alanine.

Preferably, the term “variants” as normally understood in the present invention refers to variants with increased promiscuity, wherein promiscuity is understood as the ability of the intein to perform the protein trans-splicing (PTS) reaction independently of the identity of the residues immediately adjacent to the split site. More specifically, inteins promiscuity refers to their ability to perform the PTS reaction independently of the identity of the amino acids immediately adjacent to the split site, that is, the site where the intein is inserted in the protein of interest. Below is shown the schematic representation of a split site:
−3−2 −1 +1+2+3
X-X-X-Intein N Intein C-X-X-X

Typically, inteins have strong preferences for certain amino acids in those positions. For example, the Cat intein (SEQ ID NO 30) favors a Cys at position +1, and a Glu at position −1 (Stevens, A. J., Sekar, G., Gramespacher, J. A., Cowburn, D., & Muir, T. W. (2018)). An Atypical Mechanism of Split Intein Molecular Recognition and Folding. Journal of the American Chemical Society, 140(37), 11791-11799. http://doi.org/10.1021/jacs.8b07334). More promiscuous variants would be those able to perform the protein trans-splicing reaction in good yields even in the absence of such preferred residues.

In a particular embodiment, intein variants include mutants in which the catalytic and second shell accelerator residues of the intein are maintained, and only non-catalytic residues, or residues outside the second shell are mutated. Second shell accelerator residues are those adjacent to the active site of the intein, which play a critical role in tuning the splicing activity of the inteins. For example, catalytic and accelerator residues of the Cfa inteins, and inteins of the DnaE family, are well known and have been described in the art (Stevens, A. J., Brown, Z. Z., Shah, N. H., Sekar, G., Cowburn, D., & Muir, T. W. (2016). Journal of the American Chemical Society. http://doi.org/10.1021/jacs.5b13528). Similarly, catalytic residues for multiple other intein families, including DnaE, GyrA, GyrB, DnaB, TerL, gp41, IMPDH, have been studied and characterized (Shah, N. H., & Muir, T. W. (2014). Inteins: Nature's Gift to Protein Chemists. Chemical Science (Royal Society of Chemistry: 2010), 5(1), 446-461. http://doi.org/10.1039/C3SC52951G). Methodologies to identify second shell accelerator residues have also been described (Stevens et al. 2016 Journal of the American Chemical Society. http://doi.org/10.1021/jacs.5b13528) and could be used to generate intein variants (both N-intein and C-inteins) with suitable properties to be used in the invention.

In a particular embodiment, intein variants include mutants in which the catalytic residues of the intein are maintained, and the variant retains key functional features of CfaN, NpuN, CatN, or gp41N inteins, including fast splicing rates (half-lives below 5 min), high activity in the presence of chaotropic agents or high temperature. In a more particular embodiment, the variant of the CfaN, CatN, NpuN or gp41N inteins is a functionally equivalent variant of any of these sequences.

The term “functionally equivalent variant” as used herein is understood to mean all those proteins derived from a sequence by modification, insertion and/or deletion or one or more amino acids, whenever the function is substantially maintained or improved, particularly in the case of a functionally equivalent variant of the split intein N-fragment refers to maintaining its activity. The term “activity” as used herein refers to the ability of the split intein N-fragment to perform a protein trans-splicing reaction upon binding to a split intein C-fragment.

Examples of functionally equivalent variants of CfaN, NpuN, CatN, or gp41N inteins are indicated as follows:
CfaN functionally equivalent variants can be selected from any of the following list consisting of:

The CfaN sequence (SEQ ID NO 27) in which Cys28 and/or Cys59 are mutated to Ser, Thr or Ala.

The CfaN sequence (SEQ ID NO 27) in which additional amino acids are included at the N-terminus of the Met1. For example, a linker, a degron, or a tag for detection.

The CfaN sequence (SEQ ID NO 27) in which any of its residues is mutated to a residue with similar physicochemical properties, except for the following residues which must be maintained: Cys1, Lys70, His72, Met75, Met81.

The CfaN sequence (SEQ ID NO 27) in which any of its residues is mutated to a residue with similar physicochemical properties, except for the following residues which must be maintained: Cys1, Asp5, Phe15, Glu24, Thr32, Lys35, Phe38, Val39, Ile44, Asn49, Ile65, Thr69, Lys70, His72, Met75, Thr77, Met81, Gly91, Lys95, Gln96, Gly99.

The CfaN sequence (SEQ ID NO 27) in which the following residues are maintained: Cys1, Lys70, His72, Met75, Met81, and any of the following mutations or any combination thereof is introduced: Asp5Glu, Phe15Leu, Glu24Lys, Thr32Ser, Lys35Asn, Phe38Asn, Val39Ile, Ile44Val, Asn49Asp, Ile65Leu, Thr77Val, Gly91Glu, Lys95Met, Gln96Arg, Gly99Asn.

The CfaN sequence (SEQ ID NO 27) in which the following residues are maintained: Cys1, Lys70, His72, Met75, Met81, and from 1 to 6 (wherein 1 and 6 are included within the range) of the following mutations are introduced Asp5Glu, Phe15Leu, Glu24Lys, Thr32Ser, Lys35Asn, Phe38Asn, Val39Ile, Ile44Val, Asn49Asp, Ile65Leu, Thr77Val, Gly91Glu, Lys95Met, Gln96Arg, Gly99Asn.

The CfaN sequence (SEQ ID NO 27) in which the following residues are maintained: Cys1, Asp5, Glu24, Phe38, Val39, Ile44, Lys70, His72, Met75, Met81, Gly91, Lys95, Gln96, Gly99 and any of the following mutations, or combination of mutations are introduced Phe15Ala, Thr32Ser, Lys35Glu, Asn49Asp, Ile65Thr, Thr77Glu.
NpuN functionally equivalent variants can selected from any of the following list consisting of:

The NpuN sequence (SEQ ID NO 32) in which Cys28 and/or Cys59 are mutated to Ser, Thr or Ala.

The NpuN sequence (SEQ ID NO 32) in which any residue of NpuN can be mutated to a residue with similar physicochemical properties, except for the following residues which must be maintained: Cys1, Thr69, Lys70, His72, Met75, Met81.

The NpuN sequence (SEQ ID NO 32) in which any residue of NpuN can be mutated to the corresponding amino acid in the sequence of CfaN.
Gp41N functionally equivalent variants can selected from any of the following list consisting of:

The Gp41N sequence (SEQ ID NO 38) in which Cys59 and/or Cys83 are mutated to Ser, Thr or Ala.

The Gp41N sequence (SEQ ID NO 38) in which any residue of Gp41N can be mutated to a residue with similar physicochemical properties, except for the following residues which must be maintained: Cys1, His63, and residue 60 is a Ser or a Thr.
CatN functionally equivalent variants can be selected from any of the following list consisting of:

The CatN sequence (SEQ ID NO 30) in which any residue of CatN can be mutated to a residue with similar physicochemical properties, except for the Cys1 residue which must be maintained.

In a particular embodiment, the Intein N comprises or consists of a variant of the amino acid sequence of SEQ ID NO: 27, SEQ ID NO 30, SEQ ID 32, or SEQ ID NO 38 having at least 90% sequence identity with SEQ ID NO: 27, SEQ ID NO 30, SEQ ID 32, or SEQ ID NO 38, respectively, over the whole sequence. In a particular embodiment, the variant of the Intein N of SEQ ID NO: 27, SEQ ID NO 30, SEQ ID 32, or SEQ ID NO 38, has at least 91%, at least 92%, at least 93%, at least 94%, at least 95%, at least 96%, at least 97%, at least 98%, or at least 99% sequence identity with SEQ ID NO: 27, SEQ ID NO 30, SEQ ID 32, or SEQ ID NO 38, respectively, over the whole sequence.

The terms “identity”, “identical”, “percent identity” or “sequence identity” in the context of two or more amino acid or nucleotide sequences, refer to two or more sequences or subsequences that are the same or have a specified percentage of amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid sequences. One such non-limiting example of a sequence alignment algorithm is the algorithm described in Karlin et al., 1990, Proc. Natl. Acad. Sci., 87:2264-8, as modified in Karlin et al., 1993, Proc. Natl. Acad. Sci., 90:5873-7, and incorporated into the N BLAST and XBLAST programs (Altschul et al., 1991, Nucleic Acids Res., 25:3389-402). In certain embodiments, Gapped BLAST can be used as described in Altschul et al., 1997, Nucleic Acids Res. 25:3389-402. BLAST-2, WU-BLAST-2 (Altschul et al., 1996, Methods in Enzymology, 266:460-80), ALIGN, ALIGN-2 (Genentech, South San Francisco, Calif.) or Megalign (DNASTAR) are additional publicly available software programs that can be used to align sequences. In certain alternative embodiments, the GAP program in the GCG software package, which incorporates the algorithm of Needleman and Wunsch (J. Mol. Biol. 48:444-53 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between amino acid sequences is determined using the algorithm of Myers and Miller (CABIOS, 4:1 1-7 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second amino acid sequence is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the second sequence is longer than the first sequence, then the global alignment taken the entirety of both sequences into consideration is used, therefore all letters and null in each sequence must be aligned. In this case, the same formula as above can be used but using as Z value the length of the region wherein the first and second sequence overlaps, said region having a length which is substantially the same as the length of the first sequence.

As a non-limiting example, whether any particular polypeptide has a certain percentage of sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 5371 1). Bestfit uses the local homology algorithm of Smith and Waterman, Advances in Applied Mathematics 2:482-9 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference amino acid sequence and that gaps in homology of up to 5% of the total number of nucleotides in the reference sequence are allowed.

The term “linker” between the split intein N-fragment and the degron refers to a sequence of amino acids that connect the C-terminus of the intein N-fragment and the N-terminus of the degron sequence. The linker would preferably be a polypeptide of 1 to 100 amino acids, or 1 to 5, or 1 to 10, or 1 to 50, or 1 to 25 amino acids. The linker could be a Gly rich peptide, or a Gly-Ser rich peptide. An example of a Gly-Ser rich peptide would be the sequence GGS, or polymers of this linker with the general formula (GGS)n, where n can go from 1 to 10. A general formula for the linker would be ((G)nS)y, where n goes from 1 to 5 and y from 1 to 10. Additionally, epitope tags can be used as linkers, for example the hexahistidine (His6, sequence: GHHHHHHG (SEQ ID NO 84) tag, or a triple flag tag (3FT, sequence:

| (SEQ ID NO 103) | |
|---|---|
| | DYKDHDGDYKDHDIDYKDDDDK). |

In another particular embodiment, the degron directly linked via a peptide bond to the split intein N-fragment is selected from any of those listed in table 3 below:

TABLE 3

| | | SEQ |
|---|---|---|
| | | ID |
| Degron | Sequence | NO |
| CL1 | ACKNWFSSLSHFVIHL | 40 |
| Deg1 | MNKIPIKDLLNPQITDEFKSSILDINKKLFSICCNLP | 41 |
| | KLPESVTTEEEVELRDILGFLSRAN | |
| PEST | RSSSPSDSDTSGFSSGSDHLSDLISSLRIS | 42 |
| DD1 | RISFGSPPPMAXG; X = any amino acid, | 43 |
| | preferably Gly (G) | |
| DD2 | GSPPPMAXG; X = any amino acid, | 44 |
| | preferably Gly (G) | |
| DD3 or | TNGILKLGCQG | 45 |
| VI5 | | |
| M1 | CSEIIPMSRSTPISTMG | 46 |
| M2 | VSFAFNLNSLIVGILRFHW | 47 |
| SopE or | MTKITLSPQNFRIQKQETTLLKEKSTEKNSLAKSILA | 48 |
| SopE100 | VKNHFIELRSKLSERFISHKNTESSATHFHRGSASEG | |
| | RAVLTNKVVKDFMLQTLNDIDIRGSA | |
| SopE-1- | MTKITLSPQNFRIQKQETTLLKEKSTEKNSLAKSILA | 49 |
| 78 | VKNHFIELRSKLSERFISHKNTESSATHFHRGSASEG | |
| | RAVL | |
| SopE-15- | QETTLLKEKSTEKNSLAKSILAVKNHFIELRSKLSER | 50 |
| 78 | FISHKNTESSATHFHRGSASEGRAVL | |
| SopE-15- | QETTLLKEKSTEKNSLAKSILAVKNHFIELRSKLS | 51 |
| 50 | | |
| L2 or | SLISLPLPTRVKFSSLLLIRIMKIITMTFPKKLRS | 52 |
| CL2 | | |
| L6 | FYYPIWFARVLLVHYQ | 53 |
| L9 or | SNPFSSLFGASLLIDSVSLKSNWDTSSSSCLISFFSS | 54 |
| CL9 | VMFSSTTRS | |
| L10 | CRQRFSCHLTASYPQSTVTPFLAFLRRDFFFLRHNSS | 55 |
| | AD | |
| L11 | GAPHVVLFDFELRITNPLSHIQSVSLQITLIFCSLPS | 56 |
| | LILSKFLQV | |
| L12 or | NTPLFSKSFSTTCGVAKKTLLLAQISSLFFLLLSSNI | 57 |
| CL12 | AV | |
| L15 | PTVKNSPKIFCLSSSPYLAFNLEYLSLRIFSTLSKCS | 58 |
| | NTLLTSLS | |
| L16 | SNQLKRLWLWLLEVRSFDRTLRRPWIHLPS | 59 |
| M3 | KSVTLESRSPKFLNWFSVFSLFKVITTG | 34 |
| M4 | YMSILRCASGKISIAAPPYIF | 35 |
| M5 | AGESFNFMVKLLYKHPILPCLKTLLSIRSSCSPR | 37 |
| VI2 | TNGSVLREFTLLELEVVTRNTEL | 85 |
| DD4 | RDSFGSPPPMAXG; X = any amino acid, | 80 |
| | preferably Gly (G) | |
| DD5 | GFWI | 81 |
| DD6 | RFKG | 82 |
| DD7 | KFYK | 83 |

Other non-limiting examples of degrons include polypeptides related to DHFR (dehydrofolate reductase), FKBP (FK506 Binding Protein), FRB (FKBP-Rapamycin binding protein) and PDE5 (phsophodiesterase type 5). As well as degradation signals of the Endoplasmic Reticulum Associated Degradation (ERAD) pathway.

In yet another preferred embodiment the degron is a polypeptide of less than 75 amino acids, which once fused to the intein N fragment of the invention it induces its degradation.

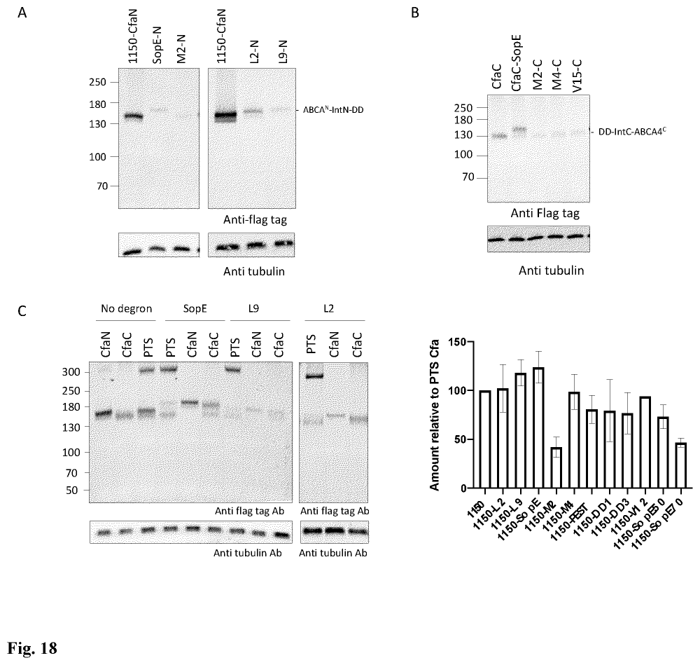

In yet another preferred embodiment the degron is a polypeptide which when fused to the the split intein N- or C-terminal fragment of the invention, it results in a reduction of the expression levels of said fragment by more than 10%, or more than 20%, or more than 30%, or more than 50%, or more than 75%, or more than 90%. Importantly, the incorporation of the degron in the fragments of the invention does not significantly affect the yield of the reconstituted protein resulting from the protein trans-splicing between the split intein N- and C-terminal fragment degron of the invention. For the purposes of this preferred embodiment, the expression levels are measured as described in FIG. 18, that is the expression levels of the fragments with degron are compared with those of the fragments without degron, under conditions analogous to the ones described in FIG. 18, and the former needs to be at least 10%, or at least 20%, or at least 30%, or at least 50%, or at least 75%, or at least 90%, lower than the latter.

In yet another preferred embodiment, preferred combinations of N inteins and degrons of the split intein N-fragment degron of the invention are selected from the list consisting of:
CfaN or any functionally equivalent variant thereof in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7. Preferably, CfaN or any functionally equivalent variant thereof in combination with any of the following degrons: CL1, Deg1, DD1, DD2, DD3, SopE, L2, L9, M4, V12, or DD4; or
Gp41N or any functionally equivalent variant thereof in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7. Preferably, Gp41^{N}, or any functionally equivalent variant thereof, in combination with any of the following degrons: CL1, Deg1, DD1, DD2, DD3, SopE, L2, L9, M4, V12, or DD4.

In FIG. 18A it is shown the effect that some representative degrons have when fused to a split N-intein fragment, in particular to the CfaN intein. The addition of the degrons (SopE, L2, L9, M4, V12) causes a significant reduction in the detectable expression levels of the N-intein containing protein fusion to which the degrons are fused. Interestingly, as described in the examples, and in FIGS. 18, 20 and 21, the inclusion of some preferred degrons (SopE, L2, L9, M4, or V12) does not negatively affect the yield of splicing product formation. This result was not anticipated by the state of the art, as, when reducing the levels of starting materials for a protein trans-splicing reaction, as accomplished by the addition of the degrons, one would expect a reduction in the protein splicing yield.

In yet another preferred embodiment, preferred combinations of split intein N-fragments and degrons are selected from the list consisting of: the CfaN intein with any of the following degrons DD1, DD2, DD3, SopE, L2, L9, M4, V12, DD4, DD5, DD6 or DD7.

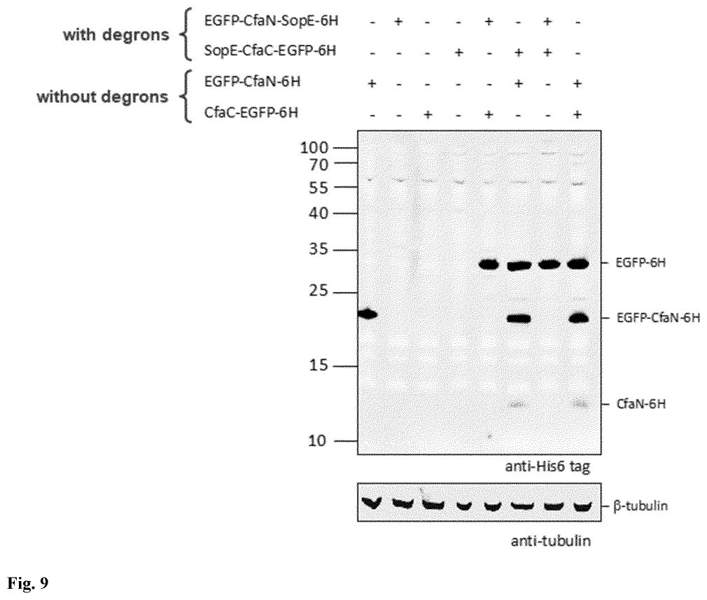

Thus, we have observed that when the CfaN intein is combined with any of the above mentioned degrons, particularly the following: DD1, DD2, DD3, SopE, L2, L9, M4, V12, DD4, DD5, DD6 or DD7, a reduction on the expression levels of the N-terminal intein fragment is detected (FIG. 9, 18A). Importantly, this does not affect the yields of the protein trans-splicing reaction (FIG. 9, 18C, 20 or 21). This result is unexpected, based on the prior art, as typically, a reduction on the yield, expression, or stability of one of the two intein fragments involved in a protein trans-splicing reaction results in an overall reduction of the amount of splicing product formed.

We have observed this effect for a membrane protein such as ABCA4 (FIGS. 18, 20 and 21), as well as for a cytosolic soluble protein, such as EGFP, illustrating the generality of the effect. In FIG. 9 it can be observed how the addition of a degron to the EGFPN-CfaN, results in the complete disappearance of the band corresponding to that protein. However, upon addition of the CfaC-EGFPC fragment (with, or without degron) the full-length EGFP product is formed with the same yields than if no degrons were used. This example illustrates that both the addition of one, or even two degrons (one on each fragment), do not negatively affect the yields of the target protein.

Complex Comprising a Split Intein N-Fragment

In another aspect, the invention relates to any of the following complex/es: the split intein N-fragment of the invention or the split intein N-fragment degron of the invention, hereinafter first complex/es of the invention, wherein each of these complexes comprises:

(i) the N-terminal fragment of a protein of interest to be reconstituted, and

(ii) a split intein N-fragment or a split intein N-fragment directly linked via a peptide bond to a degron, optionally through a linker, as defined in the above section (the split intein N-fragment degron of the invention);

wherein the complex/es optionally comprise a linker between (i) and (ii) and

wherein
the N-terminal fragment of the protein of interest is linked to the N-terminus of the split intein N-fragment by an amide linkage, or
if the complex/es comprise a linker, the N-terminal fragment of the protein of interest is bound to the linker by an amide linkage and/or the linker is bound to the N-terminus of the split intein N-fragment by an amide linkage.

Illustrative non-limitative examples of proteins of interest, useful in the any of the above complexes, are those shown in table 1 above. Further proteins of interest can be selected from antibodies, antibody fragments, including Fc domain, scFv, nanobodies, bi-specific antibodies, proteins, and, preferably, any proteins larger than 25, 50, 100 KDa.

As already indicated, optionally, the proteins of interest and the split intein N-fragment may be joined through a linker, so the linker is located in between the protein of interest and the N-intein. The nature of the linker will depend on the nature of the protein of interest. In a particular embodiment, the linker is a peptide. In a particular embodiment, the linker is a peptide having a length of 1, 2, 3, 4, 5, 10, 20, 50, 100 or more amino acid residues; specifically, it may be 1 to 3 amino acid residues. Preferably, the N-terminus of the linker is linked to the C-terminus of the protein of interest and the C-terminus of the linker is linked to the N-terminus of the N-intein through peptide bonds.

In another particular embodiment, the complex does not comprise a linker between the N-terminal fragment of a protein of interest and the split intein N-fragment. In this particular embodiment, the protein of interest is linked to the N-terminus of the split intein N-fragment by an amide linkage.

In another particular embodiment, if the complexes comprise a linker, the complex is a fusion protein. The term “fusion protein” is well known in the art, referring to a single polypeptide chain artificially designed which comprises two or more sequences from different origins, natural and/or artificial. The fusion protein, per definition, is never found in nature as such.

Split Intein C-Fragment

In another aspect, the invention relates to a Split intein C-fragment directly linked via a peptide bond to the C-terminal fragment of a protein to be reconstituted (from hereinafter “the split intein C-fragment of the invention”).

More preferably, the invention refers to a split intein C-fragment directly linked via a peptide bond to a degron (from hereinafter “the split intein C-fragment degron of the invention”), wherein the degron is linked to the intein C-fragment via the N-terminus of the intein, with or without a linker between the intein C-fragment and the degron, and wherein the C-terminus of the Split intein C-fragment is directly linked via a peptide bond to the N-terminus of the C-terminal fragment of a protein to be reconstituted.

Therefore, the architecture of the construct of the split intein C-fragment degron of the invention, from the N to the C-terminus would be:

(Degron)-(Intein-C fragment)-(C-terminal portion of the protein to be reconstituted)

If a linker is included between Degron and Intein C, the architecture of the construct of the split intein C-fragment degron of the invention, from the N to the C-terminus would be:

(Degron)-(linker)-(Intein-C fragment)-(C-terminal portion of the protein to be reconstituted)

Preferably, the Intein C of any of the split intein C-fragment of the invention or of the split intein C-fragment degron of the invention is selected from any of the following listed in table 4 below:

TABLE 4

| | | SEQ ID |
|---|---|---|
| Name | Sequence | NO |
| Cfa^{C} | MVKIISRKSLGTQNVYDIGVEKDHNFLLKNGLVASN | 28 |
| Cfa^{Cmut} | MVKIISRKSLGTQNVYDIGVGEPHNFLLKNGLVASN | 29 |
| Cat^{C} | MFKLNTKNIKVLTPSGFKSFSGIQKVYKPFYHHIIF | 31 |
| | DDGSEIKCSDNHSFGKDKIKASTIKVGDYLQGKKVL | |
| | YNEIVEEGIYLYDLLNVGEDNLYYTNGIVSHN | |
| Npu^{C} | MIKIATRKYLGKQNVYDIGVERDHNFALKNGFIASN | 33 |
| Npu^{Cmut} | MIKIATRKYLGKQNVYDIGVGEPHNFALKNGFIASN | 36 |
| Gp41C | MMLKKILKIEELDERELIDIEVSGNHLFYANDILTH | 104 |
| | N | |
| ConC | MVKIISRQSLGKQNVYDIGVEKDHNFLLANGLIASN | 105 |

wherein preferably the Intein C is the CfaC intein of SEQ ID NO 28 or any variants thereof such as CfaCmut (SEQ ID NO 29) or ConC (SEQ ID NO 105); or the CatC intein of SEQ ID NO 31 or any variants thereof, or the NpuC intein (SEQ ID NO 33) or any variants thereof such as NpuCmut (SEQ ID NO 36), or the GP41C intein of SEQ ID NO 104 or any variants thereof; or any variants with increased promiscuity as defined above.

The term variant is as defined for the split intein N-fragment. Variants of the Intein C include SEQ ID NO 28, 29, 31, 33, 36, 104 and 105 without the N-terminal Methionine residue.

In a particular embodiment the CfaC, NpuC, or ConC variants with increased promiscuity contain the amino acids Gly, Glu and Pro, in positions 21, 22 and 23, as described in Stevens et al. Proc. Natl. Acad. Sci. 2017.

In a particular embodiment, examples of functionally equivalent variants of Cfac, NpuC CatC or gp41c inteins are indicated as follows:
CfaC functionally equivalent variants can be selected from any of the following list consisting of:

The CfaC (SEQ ID NO 28) sequence in which Met1 is removed.

The CfaC (SEQ ID NO 28) sequence in which additional amino acids are included at the N-terminus Met1. For example, a linker, a degron, or a tag for detection.

The CfaC (SEQ ID NO 28) sequence in which any residue of CfaC is mutated to a residue with similar physicochemical properties except the following residues which should be maintained: Asp17, His24, Asn36.

The CfaC (SEQ ID NO 28) sequence in which any residue of CfaC is mutated to a residue with similar physicochemical properties, except the following residues which should be maintained: Asp17, Asp23, His24, Ser35, Asn36.

The CfaC (SEQ ID NO 28) sequence in which any residue of CfaC is mutated to a residue with similar physicochemical properties, except the following residues which should be maintained: Asp17, Asp23, His24, Ser35, Asn36.

The CfaC (SEQ ID NO 28) sequence in which the following residues are maintained: Asp17, Asp23, His24, Ser35, Asn36, and any of the following amino acids is mutated Val2, Ile5, Serb, Ser9, Lys22, Leu27, Leu32, Val33.

The CfaC (SEQ ID NO 28) sequence in which the following residues are mutated: Glu21Gly, Lys22Glu, and Asp23Pro. This variant corresponds to the CfaCmut intein (SEQ ID NO 29).

The CfaC (SEQ ID NO 28) sequence in which any residue is mutated to a residue with similar physicochemical properties, except the following residues which should be at the indicated positions: Asp17, Gly21, Glu22, Pro23, His24, Asn36.

The CfaC (SEQ ID NO 28) sequence in which the following residues should be at the indicated positions: Asp17, Gly21, Glu22, Pro23, His24, Ser35, Asn36, and any of the following amino acids are mutated Val2, Ile5, Ser9, Leu27, Leu32, Val33.

The CfaC (SEQ ID NO 28) or CfaCmut (SEQ ID NO 29) sequence, in which from 1 to 5 (wherein 1 and 5 are included within the range) of the following mutations are introduced Val2Ile, Ile5Ala, Ser6Thr, Ser9Tyr, Thr12Lys, Leu27Ala, Leu32Phe, Val33Ile.
NpuC functionally equivalent variants can be selected from any of the following list consisting of:

The NpuC (SEQ ID NO 33) sequence in which in which Met1 is removed.

The NpuC (SEQ ID NO 33) sequence in which additional amino acids are included at the N-terminus of the Met1. For example, a linker, a degron, or a tag for detection.

The NpuC (SEQ ID NO 33) sequence in which any residue of NpuC can be mutated to a residue with similar physicochemical properties, except the following residues which should be maintained: Asp17, His24, Asn36.

The NpuC (SEQ ID NO 33) sequence in which any residue of NpuC can be mutated to a residue with similar physicochemical properties, except the following residues need to be maintained: Asp17, His24, Ser35, Asn36.

The NpuC (SEQ ID NO 33) sequence in which the following residues are maintained: Asp17, His24, Ser35, Asn36, and any of the following amino acids is mutated Val2, Ile5, Ser9, Leu27, Leu32, Val33.

The NpuC (SEQ ID NO 33) sequence in which the following residues are maintained: Asp17, His24, Ser35, Asn36, and any of the following mutations is incorporated Ile2Val, Ala5Ile, Tyr9Ser, Ala27Leu, Phe32Leu, Ile33Val.

The NpuC (SEQ ID NO 33) sequence in which the following residues are mutated Glu21Gly, Arg22Glu, Asp23Pro, to increase the promiscuity of Npu.
Gp41C functionally equivalent variants can selected from any of the following list consisting of:

The Gp41C (SEQ ID NO 104) sequence in which Met1 is removed.

The Gp41C (SEQ ID NO 104) sequence in which additional amino acids are included at the N-terminus of the Met1. For example, a linker, a degron, or a tag for detection.

The Gp41C (SEQ ID NO 104) sequence in which any residue of Gp41C can be mutated to a residue with similar physicochemical properties, except the following residues need to be maintained: His26, His36, Asn37.

In a particular embodiment, the Intein C comprises or consists of a variant of the amino acid sequence of SEQ ID NO: 28, SEQ ID NO: 31, SEQ ID NO: 33, or SEQ ID NO: 104 having at least 90% sequence identity with SEQ ID NO: 28, SEQ ID NO: 31, SEQ ID NO: 33, or SEQ ID NO: 104, respectively, over the whole sequence. In a particular embodiment, the variant of the Intein C of SEQ ID NO: 28, SEQ ID NO: 31, SEQ ID NO: 33, or SEQ ID NO: 104, has at least 91%, at least 92%, at least 93%, at least 94%, at least 95%, at least 96%, at least 97%, at least 98%, or at least 99% sequence identity with SEQ ID NO: 28, SEQ ID NO: 31, SEQ ID NO: 33, or SEQ ID NO: 104, respectively, over the whole sequence.

In another particular embodiment, the degron directly linked via a peptide bond to the split intein C-fragment is selected from any of those listed in table 3. Illustrative non-limitative examples of proteins of interest, useful in this section, are those shown in table 1 above.

In yet another preferred embodiment, preferred combinations of C inteins and degrons of the split intein C-fragment degron of the invention are selected from the list consisting of:
CfaC or any functionally equivalent variant thereof, such as CfaCmut, in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7. Preferably, CfaC or any functionally equivalent variant thereof, such as CfaCmut, in combination with any of the following degrons: CL1, Deg1, DD1, DD2, DD3, SopE, L2, L9, M4, V12, DD4, DD5, DD6 or DD7; or
Gp41C or any functionally equivalent variant thereof in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7. Preferably, Gp41C or any functionally equivalent variant thereof in combination with any of the following degrons: CL1, Deg1, DD1, DD2, DD3, SopE, L2, L9, M4, V12, or DD4.

In FIG. 18B, the effect of fusing degrons to the C-terminal construct can be observed. As observed for the N-terminal degrons, fusion of degrons to the C-terminal construct, reduces the expression levels of the construct, but no effect on the splicing yields is observed, when preferred combinations are used. The following degrons work particularly well with CfaC: DD1, DD2, DD3, SopE, L2, L9, M4, V12, DD4, DD5, DD6 or DD7. As observed for the N-terminal fragments, incorporation of a degron to the C-terminal fragment of the invention resulted in a reduction of its expression (as detected by WB), but again, interestingly, no negative effect on the PTS yields, that is the formation of the desired product was observed. This result has been observed for several proteins, including the examples shown here for EGFP and ABCA4.

Complex Comprising a Split Intein C-Fragment

In another aspect, the invention relates to any of the following complex/es: the split intein C-fragment of the invention or the split intein C-fragment degron of the invention, hereinafter second complex/es of the invention, wherein each of these complexes comprises:

(i) the C-terminal fragment of a protein of interest, and

(ii) a split intein C-fragment or a split intein C-fragment directly linked via a peptide bond to a degron, optionally through a linker, as defined in the above section (the split intein C-fragment degron of the invention);

wherein the complex/es optionally comprise a linker between (i) and (ii) and

wherein
the C-terminal fragment of the protein of interest is linked to the C-terminus of the split intein C-fragment by an amide linkage, or
if the complex/es comprise a linker, the C-terminal fragment of the protein of interest is bound to the linker by an amide linkage and/or the linker is bound to the C-terminus of the split intein C-fragment by an amide linkage.

Illustrative non-limitative examples of proteins of interest, useful in the present invention, are those listed in table 1 above. Further examples of proteins of interest can be selected from antibodies, antibody fragments, including Fc domain, scFv, nanobodies, bi-specific antibodies, proteins, and, preferably, any proteins larger than 25, 50, 100 KDa.

The terms “protein of interest” and “linker” have been previously defined in connection with the first complex of the invention. All the particular embodiments of the protein of interest and linker of the first complex of the invention fully apply to the second complex of the invention.

In a particular embodiment, the complex does not comprise a linker between the compound of interest and the split intein C-fragment. In this particular embodiment, the compound of interest is linked to the C-terminus of the split intein C-fragment by an amide linkage.

In a particular embodiment, the complex comprises a linker between the compound of interest and the split intein C-fragment. In this particular embodiment, the compound of interest may be bound to the linker by any suitable means, depending on the chemical nature of the compound of interest and of the linker. In this particular embodiment, the linker is bound to the C-terminus of the split intein C-fragment by an amide linkage. In another particular embodiment, the compound of interest is bound to the linker by an amide linkage, in which case the linker may be bound to the C-terminus of the split intein C-fragment by any suitable means. In another particular embodiment, the compound of interest is bound to the linker by an amide linkage and the linker is bound to the C-terminus of the split intein C-fragment by an amide linkage.

In a particular embodiment, if the complex comprises a linker, the linker is a peptide linker. In this particular embodiment, the complex is a fusion protein.

Composition Comprising the Complexes of the Invention

In another aspect, the invention refers to a composition, hereinafter first composition of the invention, comprising the first and/or the second complex/es of the invention.

The term “composition” is intended to encompass a product containing the specified components, as well as any product that results, directly or indirectly, from a combination of the specified components in the specified amounts. The components of the composition may be packed together in a single formulation or separately in different formulations. Thus, in an embodiment, the first complex of the invention is packed together with the second complex of the invention in a single formulation. In another embodiment, the first complex of the invention and of the second complex of the invention are separately packed.

In a particularly preferred embodiment, the first and the second complex comprise the N-terminal fragment and the C-terminal fragment of the same protein respectively, in such a way that when both complexes are combined according to the methods of the invention, the N-terminal fragment of the protein is linked to the C-terminal fragment of the protein generating the whole protein.

Polynucleotides, Vectors and Host Cells of the Invention

In another aspect, the invention relates to polynucleotides encoding the first, or second complex/es of the invention.

Preferably, in a preferred embodiment, the invention refers to two polynucleotides, wherein one of these polynucleotides encodes the first complex/es of the invention (hereinafter first polynucleotide of the invention), and the other encodes the second complex/es of the invention (hereinafter second polynucleotide of the invention), wherein the first and the second polynucleotide of the invention encode the N-terminal fragment and the C-terminal fragment of the same protein respectively, in such a way that when both polynucleotides are translated into their respective protein complexes and or when combined according to the methods of the invention, the N-terminal fragment of the protein is linked to the C-terminal fragment of the protein thus generating the whole protein to be reconstituted. It is important to note that the first and second polynucleotides of the invention each preferably encodes split inteins of the same intein, that is, the N-intein and the C-inteins are cognate pairs (see Shah et al. Journal of the American Chemical Society 2012 https://doi.org/10.1021/ja303226x). Examples of cognate inteins pairs include the CfaN/CfaC (or any of their variants including CfaCmut), NpuN/NpuC (or any of their variants), CatN/CatC (or any of their variants), Gp41N/Gp41C (or any of their variants). However, independently of the fact of whether both polynucleotides encode split inteins pertaining to the same intein or not, once translated into a protein, the N-terminal and C-terminal sequences of each of the split inteins must become separate fragments that can non-covalently re-associate, or reconstitute, into an intein that is functional for trans-splicing reactions.

In a preferred embodiment, the invention refers to the two polynucleotides referred to above, wherein one of these polynucleotides encodes the split intein N-fragment degron of the invention (hereinafter first polynucleotide encoding degron of the invention), and the other encodes the split intein C-fragment degron of the invention (hereinafter second polynucleotide encoding degron of the invention), wherein the first and the second polynucleotide encoding degrons of the invention encode the N-terminal fragment and the C-terminal fragment of the same protein respectively, in such a way that when both polynucleotides are translated into their respective protein complexes and combined according to the methods of the invention, the N-terminal fragment of the protein is linked to the C-terminal fragment of the protein thus generating the whole protein. As with the previous embodiment, it is important to note that the first and second polynucleotides encoding degrons of the invention each preferably encode split inteins of the same intein. At any rate and as reflected above, as used herein each polynucleotide must encode a split intein so that, once translated into a protein, the N-terminal and C-terminal sequences become separate fragments that can non-covalently re-associate, or reconstitute, into an intein that is functional for trans-splicing reactions.

In yet another preferred embodiment, the invention refers to a three-piece ligation strategy using orthogonal split intein pairs. In this methodology, there are three polynucleotides, wherein the first polynucleotide encodes the POI^{N}-CfaN (wherein POIN is understood as the N fragment of a “protein of interest); wherein the second polynucleotide encodes the CfaC-POI^{M}-CatN (wherein the POI^{M} in this case, is an intermediate fragment of a protein of interest); and wherein the third polynucleotide encodes the CatC-POI^{C} (wherein POI^{C} is understood as the C fragment of a protein of interest). Alternatively, the positions of the Cfa and Cat inteins could be swapped, to generate constructs with the following architecture: POI^{N}-CatN, CatC-POI^{M}-CfaN and CfaC-POI^{C}. CfaN is the CfaN intein fragment SEQ ID NO 27, as well as any of its variants. CatN is the CatN intein fragment SEQ ID NO 30 as well as any of its variants. The CfaC is the CfaC intein fragment SEQ ID NO 28, as well as any of its variants, including SEQ ID NO 29. The CatC is the CatC intein fragment SEQ ID NO 31, as well as any of its variants.

It is herein noted that for those embodiments referring to two polynucleotides, the first polynucleotide (or specifically the first polynucleotide encoding degron of the invention) can be any split intein N-fragment of the invention or any split intein-N degron fragment of the invention. In particular, the first polynucleotide of the invention (or specifically the first polynucleotide encoding degron of the invention) can be selected from any of the following list consisting of: SEQ ID NO 1, 3, 5, 8, 10, 12, 14, 17, 19, 21, 23, 24, 25, 66, 68, 70, 72, 74, 76 and 78. More preferably, the first polynucleotide encoding degron of the invention encodes any of the following specific combinations of inteins and degrons:
CfaN or any functionally equivalent variant thereof, such as ConN, in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7. Preferably, CfaN or any functionally equivalent variant thereof, such as ConN, in combination with any of the following degrons: CL1, Deg1, DD1, DD2, DD3, SopE, L2, L9, M4, V12, or DD4; or
Gp41N or any functionally equivalent variant thereof in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7. Preferably, Gp41N or any functionally equivalent variant thereof in combination with any of the following degrons: CL1, Deg1, DD1, DD2, DD3, SopE, L2, L9, M4, V12, or DD4.

It is herein further noted that for those embodiments referring to two polynucleotides, the second polynucleotide (or specifically the second polynucleotide encoding degron of the invention) can be any split intein C-fragment of the invention or any split intein-C degron fragment of the invention. In particular, the second polynucleotide of the invention (or specifically the second polynucleotide encoding degron of the invention) can be selected from any of the following list consisting of: SEQ ID NO 2, 4, 6, 9, 11, 13, 15, 18, 20, 22, 26, 67, 69, 71, 73, 75, 77 and 79. More preferably, the second polynucleotide encoding degron of the invention encodes any of the following specific combinations of inteins and degrons:
CfaC or any functionally equivalent variant thereof, such as CfaCmut or ConC, in combination with any of the following degrons: CL1, Deg1, PESTt, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7. Preferably, CfaC or any functionally equivalent variant thereof, such as CfaCmut, in combination with any of the following degrons: CL1, Deg1, DD1, DD2, DD3, SopE, L2, L9, M4, V12, DD4, DD5, DD6 or DD7; or
Gp41C or any functionally equivalent variant thereof in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7. Preferably, Gp41C or any functionally equivalent variant thereof in combination with any of the following degrons: CL1, Deg1, DD1, DD2, DD3, SopE, L2, L9, M4, V12, or DD4.

In a preferred embodiment, the first and the second polynucleotide encoding degron of the invention encode the N-terminal fragment and the C-terminal fragment of the ABCA4 protein respectively, in such a way that when both polynucleotides are translated into their respective protein complexes and combined according to the methods of the invention, the N-terminal fragment of the ABCA4 protein is linked to the C-terminal fragment of the ABCA4 protein thus generating the whole ABCA4 protein. More preferably, the first polynucleotide encoding degron of the invention encodes positions 1-1149, 1-1139, 1-1178, or 1-1187 of the N-terminal fragment of the ABCA4 protein and any of the following specific combinations of inteins and degrons:
CfaN or any functionally equivalent variant thereof, such as ConN, in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7. Preferably, CfaN or any functionally equivalent variant thereof, such as ConN, in combination with any of the following degrons: CL1, Deg1, DD1, DD2, DD3, SopE, L2, L9, M4, V12, or DD4; and

the second polynucleotide encoding degron of the invention encodes positions 1150-2273, 1140-2273, 1179-2273, or 1188-2273 of the C-terminal fragment of the ABCA4 protein and any of the following specific combinations of inteins and degrons:
CfaC or any functionally equivalent variant thereof, such as CfaCmut or ConC, in combination with any of the following degrons: CL1, Deg1, PESt, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7. Preferably, CfaC or any functionally equivalent variant thereof, such as CfaCmut, in combination with any of the following degrons: CL1, Deg1, DD1, DD2, DD3, SopE, L2, L9, M4, V12, DD4, DD5, DD6 or DD7.

It is important to note that since the N-terminal fragment of the ABCA4 protein must be linked to the C-terminal fragment of the ABCA4 protein thus generating the whole ABCA4 protein, when the first polynucleotide encoding degron of the invention encodes positions 1-1149, the second polynucleotide encoding degron of the invention thus encodes positions 1150-2273 of the C-terminal fragment of the ABCA4 protein. The same for the rest of the positions.

Using the preferred combinations described above it can be observed, that efficient reconstitution of the target protein, ABCA4 in these examples, can be obtained, while significantly reducing the levels of the starting N-terminal and C-terminal fragments. Inclusion of at least one degron in one of the constructs, or in both, results in a reduction of the expression, due to accelerated degradation, of the N- and/or C-terminal fragment. Based on the state of the art, it would be expected that such reduction in the levels of starting materials would result in a reduction on the splice product. Nevertheless, we observe the opposite behavior, and the yields of full-length spliced product are not affected, and in some instances can even be improved. Moreover, we observe that by reconstituting the protein using the methods described here we can generate a functional protein, as determined using ATPase activity assays. We also have observed efficient reconstitution of the target protein in vivo in mice retinas.

In another preferred embodiment, the first polynucleotide encoding degron of the invention encodes positions 1-1095, or 1-1185 of the N-terminal fragment of the ABCA4 protein and any of the following specific combinations of inteins and degrons:
Gp41N or any functionally equivalent variant thereof in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7. Preferably, Gp41N or any functionally equivalent variant thereof in combination with any of the following degrons: CL1, Deg1, DD1, DD2, DD3, SopE, L2, L9, M4, V12, or DD4; and

the second polynucleotide encoding degron of the invention encodes positions 1096-2273, or 1186-2273 of the C-terminal fragment of the ABCA4 protein and any of the following specific combinations of inteins and degrons:
Gp41C or any functionally equivalent variant thereof in combination with any of the following degrons: CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7. Preferably, Gp41C or any functionally equivalent variant thereof in combination with any of the following degrons: CL1, Deg1, DD1, DD2, DD3, SopE, L2, L9, M4, V12, or DD4.

As in the previous case, it is important to note that since the N-terminal fragment of the ABCA4 protein must be linked to the C-terminal fragment of the ABCA4 protein thus generating the whole ABCA4 protein, when the first polynucleotide encoding degron of the invention encodes positions 1-1095, the second polynucleotide encoding degron of the invention thus encodes positions, 1096-2273 of the C-terminal fragment of the ABCA4 protein. The same for the rest of the positions.

Further preferred combinations of nucleotide sequences coding for the above sequences (respectively for the first and second polynucleotides of the invention or the first and second polynucleotide encoding degrons of the invention) are selected from any of the following pairs: 1 and 2; 3 and 4; 5 and 6; 66 and 67; 68 and 69; 70 and 71; 72 and 73; 74 and 75; 76 and 77; and 78 and 79.

It is noted that the terms “first and second polynucleotides of the invention” encompass, among others, the “first and second polynucleotide encoding degrons of the invention”, and from herein below we shall thus only refer to the first and second polynucleotides of the invention as terms that already encompass all of the above-mentioned alternatives of polynucleotides.

Preferably, the invention refers to a composition, hereinafter second composition of the invention, comprising the first and/or the second polynucleotide of the invention or the first and/or the second polynucleotide encoding degrons of the invention and/or separately or jointly each of the POI-intein fragments of the three-piece ligation system using orthogonal split intein pairs. The term “composition”, in this specific context, is intended to encompass a product containing the specified components. The components of the composition may be packed together in a single formulation or separately in different formulations. Thus, in an embodiment, the first polynucleotide of the invention is packed together with the second polynucleotide of the invention in a single formulation. In another embodiment, the first polynucleotide of the invention and the second polynucleotide of the invention are separately packed. More preferably, the first and the second polynucleotides of the invention, respectively, encode the N-terminal fragment and the C-terminal fragment of the protein to be reconstituted, in such a way that when both complexes are combined according to the methods of the invention, the N-terminal fragment of the protein is linked to the C-terminal fragment of the protein generating the whole protein to be reconstituted.

All of the above-referred polynucleotides of the invention can be found isolated as such or forming part of vectors allowing the delivery and/or propagation of said polynucleotides in suitable host cells. Therefore, in another aspect, the invention relates to a vector comprising any of the above-mentioned polynucleotides of the invention as described above.

Vectors suitable for the insertion of said polynucleotides are vectors derived from expression vectors in prokaryotes such as pUC18, pUC19, Bluescript and the derivatives thereof, mp18, mp19, pBR322, pMB9, ColEl, pCRl, RP4, phages and “shuttle” vectors such as pSA3 and pAT28; expression vectors in yeasts such as vectors of the type of 2 micron plasmids, integration plasmids, YEP vectors, centromere plasmids and the like; expression vectors in insect cells such as vectors of the pAC series and of the pVL; expression vectors in plants such as pIBI, pEarleyGate, pAVA, pCAMBIA, pGSA, pGWB, pMDC, pMY, pORE series and the like; and expression vectors in eukaryotic cells, including baculovirus suitable for transfecting insect cells using any commercially available baculovirus system. The vectors for eukaryotic cells include preferably viral vectors (adenoviruses, adeno associated viruses (AAV), viruses associated to adenoviruses such as, retroviruses and, particularly, lentiviruses) as well as non-viral vectors such as pSilencer 4.1-CMV (Ambion), pcDNA3, pcDNA3.1/hyg, pHMCV/Zeo, pCR3.1, pEFI/His, pIND/GS, pRc/HCMV2, pSV40/Zeo2, pTRACER-HCMV, pUB6/V5-His, pVAXl, pZeoSV2, pCl, pSVL and PKSV-10, pBPV-1, pML2d and pTDTl. Preferably, the vectors are adeno associated viruses (AAV). Preferably the vectors are AAVs of serotype 1, 2, 3, 4, 5, 6, 7, 8, or 9. Dimeric, or self-complementary AAV vectors (scAAV), could also be used for the insertion of said polynucleotides.

The present invention is thus preferably directed to development of AAVs as gene therapy vectors. Preferably, these vectors have eliminated their integrative capacity by removal of the rep and cap from the DNA of the vector. The desired gene (polynucleotide) together with a promoter to drive transcription of the gene is inserted between the inverted terminal repeats (ITR) that aid in concatamer formation in the nucleus after the single-stranded vector DNA is converted by host cell DNA polymerase complexes into double-stranded DNA. AAV-based gene therapy vectors form episomal concatamers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency.

The desired gene (polynucleotide) might be combined with regulatory elements that are functional in the intended host cell in which the construct is to be expressed. A person of ordinary skill in the art can select regulatory elements for use in appropriate host cells, for example, mammalian or human host cells. Regulatory elements include, for example, promoters, transcription termination sequences, translation termination sequences, enhancers, signal peptides, and polyadenylation elements. The polynucleotides of the invention can be operably linked to a promoter sequence. Promoters contemplated for use in the subject invention include, but are not limited to, native gene promoters, cytomegalovirus (CMV) promoter (KF853603.1, bp 149-735), chimeric CMV/chicken beta-actin promoter (CBA) and the truncated form of CBA (smCBA) promoter (U.S. Pat. No. 8,298,818 and Light-Driven Cone Arrestin Translocation in Cones of Postnatal Guanylate Cyclase-1 Knockout Mouse Retina Treated with AAVGC 1), Rhodopsin promoter (NG 009115, bp 4205-5010), Interphotoreceptor retinoid binding protein (IRBP) promoter (NG_029718.1, bp 4777-5011), vitelliform macular dystrophy 2 (VMD2) promoter (NG 009033.1, bp 4870-5470), PR-specific human G protein-coupled receptor kinase 1 (hGRKl; AY327580.1 bpl793-2087 or bp 1793-1991) (Haire et al. 2006; U.S. Pat. No. 8,298,818), proximal murine rhodopsin promoter (MOPS). However, any suitable promoter known in the art may be used. In a specific embodiment, the promoter is a CMV or hGRKl promoter. In one embodiment, the promoter is a tissue-specific promoter that shows selective activity in one or a group of tissues but is less active or not active in other tissue. In one embodiment, the promoter is a photoreceptor-specific promoter. In a further embodiment, the promoter is a cone cell-specific and/or rod cell-specific promoter.

Preferred promoters are CMV, GRK1, CBA and IRBP promoters. Still preferred promoters are hybrid promoter which combine regulatory elements from various promoters (as example the chimeric CBA promoter which combines an enhancer from the CMV promoter, the CBA promoter and the Sv40 chimeric intron, herein called CBA hybrid promoter.

AAVs also present very low immunogenicity, seemingly restricted to generation of neutralizing antibodies, while they induce no clearly defined cytotoxic response. This feature, along with the ability to infect quiescent cells present their dominance over adenoviruses as vectors for human gene therapy.

AAV genome, transcriptome and proteome: The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

The Inverted Terminal Repeat (ITR) sequences comprise 145 bases each. They were named so because of their symmetry, which was shown to be required for efficient multiplication of the AAV genome. Another property of these sequences is their ability to form a hairpin, which contributes to so-called self-priming that allows primase-independent synthesis of the second DNA strand. The ITRs were also shown to be required for both integration of the AAV DNA into the host cell genome (19th chromosome in humans) and rescue from it, as well as for efficient encapsidation of the AAV DNA combined with generation of a fully assembled, deoxyribonuclease-resistant AAV particles.

With regard to gene therapy, ITRs seem to be the only sequences required in cis next to the therapeutic gene: structural (cap) and packaging (rep) genes can be delivered in trans. With this assumption many methods were established for efficient production of recombinant AAV (rAAV) vectors containing a reporter or therapeutic gene. However, it was also published that the ITRs are not the only elements required in cis for the effective replication and encapsidation. A few research groups have identified a sequence designated cis-acting Rep-dependent element (CARE) inside the coding sequence of the rep gene. CARE was shown to augment the replication and encapsidation when present in cis.

By 2006 11 AAV serotypes had already been described. All of the known serotypes can infect cells from multiple diverse tissue types. Tissue specificity is determined by the capsid serotype and pseudotyping of AAV vectors to alter their tropism range will likely be important to their use in therapy. In the present invention ITRs of AVV serotype 1, 2, 3, 4, 5, 6, 7, 8, or 9 are preferred.

Serotype 2 Serotype 2 (AAV2) has been the most extensively examined so far. AAV2 presents natural tropism towards skeletal muscles, neurons, vascular smooth muscle cells and hepatocytes.

The vectors may also comprise a reporter or marker gene which allows identifying those cells that have incorporated the vector after having been put in contact with it.

Useful reporter genes in the context of the present invention include lacZ, luciferase, thymidine kinase, GFP and the like. Useful marker genes in the context of this invention include, for example, the neomycin resistance gene, conferring resistance to the aminoglycoside G418; the hygromycin phosphotransferase gene, conferring resistance to hygromycin; the ODC gene, conferring resistance to the inhibitor of the ornithine decarboxylase (2-(difluoromethyl)-DL-ornithine (DFMO); the dihydrofolatereductase gene, conferring resistance to methotrexate; the puromycin-N-acetyl transferase gene, conferring resistance to puromycin; the ble gene, conferring resistance to zeocin; the adenosine deaminase gene, conferring resistance to 9-beta-D-xylofuranose adenine; the cytosine deaminase gene, allowing the cells to grow in the presence of N-(phosphonacetyl)-L-aspartate; thymidine kinase, allowing the cells to grow in the presence of aminopterin; the xanthine-guanine phosphoribosyltransferase gene, allowing the cells to grow in the presence of xanthine and the absence of guanine; the trpB gene of E. coli, allowing the cells to grow in the presence of indol instead of tryptophan; the hisD gene of E. coli, allowing the cells to use histidinol instead of histidine. The selection gene is incorporated into a plasmid that can additionally include a promoter suitable for the expression of said gene in eukaryotic cells (for example, the CMV or SV40 promoters), an optimized translation initiation site (for example, a site following the so-called Kozak's rules or an IRES), a polyadenylation site such as, for example, the SV40 polyadenylation or phosphoglycerate kinase site, introns such as, for example, the beta-globulin gene intron. Alternatively, it is possible to use a combination of both the reporter gene and the marker gene simultaneously in the same vector.

On the other hand, as the skilled person in the art knows, the choice of the vector will depend on the host cell in which it will subsequently be introduced. By way of example, the vector in which said polynucleotide is introduced can also be a yeast artificial chromosome (YAC), a bacterial artificial chromosome (BAC) or a PI-derived artificial chromosome (PAC). The characteristics of the YAC, BAC and PAC are known by the person skilled in the art. Detailed information on said types of vectors has been provided, for example, by Giraldo and Montoliu (Giraldo, P. & Montoliu L., 2001 Size matters: use of YACs, BACs and PACs in transgenic animals, Transgenic Research 10(2): 83-110). The vector of the invention can be obtained by conventional methods known by persons skilled in the art (Sambrook J. et al., 2000 “Molecular cloning, a Laboratory Manual”, 3rd ed., Cold Spring Harbor Laboratory Press, N.Y. Vol 1-3).

The polynucleotides of the invention can be introduced into the host cell in vivo as naked DNA plasmids, but also using vectors by methods known in the art, including but not limited to transfection, electroporation (e.g. transcutaneous electroporation), microinjection, transduction, cell fusion, DEAE dextran, calcium phosphate precipitation, use of a gene gun, or use of a DNA vector transporter. Methods for formulating and administering naked DNA to mammalian muscle tissue are also known. See Feigner P, et al., U.S. Pat. Nos. 5,580,859, and 5,589,466. Other molecules are also useful for facilitating transfection of a nucleic acid in vivo, such as cationic oligopeptides, peptides derived from DNA binding proteins, or cationic polymers. See Bazile D, et al., WO 1995021931, and Byk G, et al., WO 1996025508.

Another well-known method that can be used to introduce polynucleotides into host cells is particle bombardment (aka biolistic transformation). Biolistic transformation is commonly accomplished in one of several ways. One common method involves propelling inert or biologically active particles at cells. See Sanford J, et al., U.S. Pat. Nos. 4,945,050, 5,036,006, and 5,100,792.

Alternatively, the vector can be introduced in vivo by lipofection. The use of cationic lipids can promote encapsulation of negatively charged nucleic acids, and also promote fusion with negatively charged cell membranes. See Feigner P, Ringold G, Science 1989; 337:387-388. Particularly useful lipid compounds and compositions for transfer of nucleic acids have been described. See Feigner P, et al., U.S. Pat. No. 5,459,127, Behr J, et al., WO1995018863, and Byk G, WO1996017823.

Finally, and specially preferred, the vector can be introduced in vivo by viral delivery systems including but not limited to adenoviral vectors, adeno-associated viral (AAV) vectors, pseudotyped AAV vectors, herpes viral vectors, retroviral vectors, lentiviral vectors, baculoviral vectors. Pseudotyped AAV vectors are those which contain the genome of one AAV serotype in the capsid of a second AAV serotype; for example, an AAV2/8 vector contains the AAV8 capsid and the AAV 2 genome (Auricchio et al. (2001) Hum. Mol. Genet. 10(26):3075-81). Such vectors are also known as chimeric vectors. Other examples of delivery systems include ex vivo delivery systems, which include but are not limited to DNA transfection methods such as electroporation, DNA biolistics, lipid-mediated transfection, compacted DNA-mediated transfection.

The construction of an AAV vector can be carried out following procedures and using techniques which are known to a person skilled in the art. The theory and practice for adeno-associated viral vector construction and use in therapy are illustrated in several scientific and patent publications (the following bibliography is herein incorporated by reference: Flotte T R. Adeno-associated virus-based gene therapy for inherited disorders. Pediatr Res. 2005 December; 58(6):1143-7; Goncalves M A. Adeno-associated virus: from defective virus to effective vector, Virol J. 2005 May 6; 2:43; Surace E M, Auricchio A. Adeno-associated viral vectors for retinal gene transfer. Prog Retin Eye Res. 2003 November; 22(6):705-19; Mandel R J, Manfredsson F P, Foust K D, Rising A, Reimsnider S, Nash K, Burger C. Recombinant adeno-associated viral vectors as therapeutic agents to treat neurological disorders. Mol Ther. 2006 March; 13(3):463-83).

Suitable administration forms of a pharmaceutical composition containing AAV vectors include, but are not limited to, injectable solutions or suspensions, eye lotions and ophthalmic ointment. Thus, in another aspect, the invention relates to a host cell comprising the polynucleotide or the vector of the invention. The cells can be obtained by conventional methods known by persons skilled in the art (see e.g. Sambrook et al., cited ad supra).

The term “host cell”, as used herein, refers to a cell into which a nucleic acid of the invention, such as a polynucleotide or a vector according to the invention, has been introduced and is capable of expressing the split intein N-fragment of the invention or the fusion protein comprising said split intein N-fragment. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact be identical to the parent cell, but are still included within the scope of the term as used herein. The term includes any cultivatable cell that can be modified by the introduction of heterologous DNA. Preferably, a host cell is one in which the polynucleotide of the invention can be stably expressed, post-translationally modified, localized to the appropriate subcellular compartment, and made to engage the appropriate transcription machinery. The choice of an appropriate host cell will also be influenced by the choice of detection signal. For example, reporter constructs, as described above, can provide a selectable or screenable trait upon activation or inhibition of gene transcription in response to a transcriptional regulatory protein; in order to achieve optimal selection or screening, the host cell phenotype will be considered. A host cell of the present invention includes prokaryotic cells and eukaryotic cells. Prokaryotes include gram negative or gram-positive organisms, for example, E. coli or Bacilli. It is to be understood that prokaryotic cells will be used, preferably, for the propagation of the transcription control sequence comprising polynucleotides or the vector of the present invention. Suitable prokaryotic host cells for transformation include, for example, E. coli, Bacillus subtilis, Salmonella typhimurium, and various other species within the genera Pseudomonas, Streptomyces, and Staphylococcus. Eukaryotic cells include, but are not limited to, yeast cells, plant cells, fungal cells, insect cells (e.g., baculovirus), mammalian cells, and the cells of parasitic organisms, e.g., trypanosomes. As used herein, yeast includes not only yeast in a strict taxonomic sense, i.e., unicellular organisms, but also yeast-like multicellular fungi of filamentous fungi. Exemplary species include Kluyverei lactis, Schizosaccharomyces pombe, and Ustilaqo maydis, with Saccharomyces cerevisiae being preferred. Other yeasts which can be used in practicing the present invention are Neurospora crassa, Aspergillus niger, Aspergillus nidulans, Pichia pastoris, Candida tropicalis, and Hansenula polymorpha. Mammalian host cell culture systems include established cell lines such as COS cells, L cells, 3T3 cells, Chinese hamster ovary (CHO) cells, embryonic stem cells, with BHK, HeK or HeLa cells being preferred. Eukaryotic cells are, preferably, used for recombinant gene expression.

In a preferred embodiment, the second composition of the invention is for use in therapy, more particularly, for use in any of the diseases identified in table 1 depending on the type of gene coded for in the composition (a correlation between disease and gene is clearly indicated in table 1, and it is thus evident for the skilled person to identify the correct combinations).

Methods for Expressing a Gene Encoding a Protein of Interest in a Cell

In another aspect, the invention relates to a method for expressing a gene of interest in a cell, in vitro or in vivo, hereinafter first method for expressing a gene of interest, comprising:
(i) contacting the cell with
   (a) a first polynucleotide of the invention or a first polynucleotide encoding degron of the invention, and
   (b) a second polynucleotide of the invention or a second polynucleotide encoding degron of the invention,
(ii) allowing the expression of the first and the second polynucleotides so that the first and the second fusion proteins are produced and
(iii) allowing the contact between the first and second proteins so that the split intein N-fragment binds to the split intein C-fragment to form an intein intermediate and the intein intermediate reacts to covalently link the C-terminus of the first polypeptide of interest to the N-terminus of the second polypeptide of interest.

In a particular embodiment, the first polynucleotide is the first polynucleotide encoding degron and the second polynucleotide is the second polynucleotide encoding degron, wherein, preferably, the protein of interest is of more than 25 KDa, more than 50 KDa or more than 100 KDa, so that upon covalently linking the C-terminus of the first polypeptide of interest to the N-terminus of the second polypeptide of interest the whole protein is obtained.

The contacting of the cell with any of the first and/or second polynucleotides of the invention can be made, in vitro or in vivo, by any suitable means for allowing introducing a polynucleotide of interest into a cell, for example, transfection, electroporation, microinjection, transduction, lipofection, cell fusion, DEAE dextran, calcium phosphate precipitation, use of a gene gun, or use of a DNA vector transporter. Preferably, the vectors are adeno associated viruses (AAV).

In a preferred embodiment of the invention, relates to a method for expressing a gene encoding a protein of interest in a cell, hereinafter first method for expressing a gene of interest, comprising:

(i) contacting, or transducing, the cell with
(a) a first AAV comprising a first polynucleotide of the invention or a first polynucleotide encoding degron of the invention, and
(b) a second AAV comprising a second polynucleotide of the invention or a second polynucleotide encoding degron of the invention,

(ii) allowing the expression of the first and the second polynucleotides so that the first and the second fusion proteins are produced and

(iii) allowing the contact between the first and second fusion proteins so that the split intein N-fragment binds to the split intein C-fragment to form an intein intermediate and the intein intermediate reacts to covalently link the C-terminus of the first polypeptide of interest to the N-terminus of the second polypeptide of interest, wherein preferably, the first polynucleotide is the first polynucleotide encoding degron and the second polynucleotide is the second polynucleotide encoding degron, and wherein, more preferably, the protein of interest is of more than 25 KDa, more than 50 KDa or more than 100 KDa, so that upon covalently linking the C-terminus of the first polypeptide of interest to the N-terminus of the second polypeptide of interest the whole protein is obtained.

In this sense and as illustrated in the examples, the presence of degrons will cause the fast degradation of starting materials (that is the split intein N- and/or C-terminal fragment degron of the invention). Selected degrons have kinetics of degradation compatible with protein splicing in such a manner, that in the steady state the amount of starting material is reduced relative to what is observed in the absence of degrons. But the levels of splicing product are maintained, or increased, relative to what is observed in the absence of degrons.

In the method for expressing a gene of interest of the invention, it is contemplated that the cell is contacted simultaneously with the first and second polynucleotide, or sequentially with the first and second polynucleotide in any order, that is, the cell can be contacted firstly with the first polynucleotide and secondly with the second polynucleotide or firstly with the second polynucleotide and secondly with the first polynucleotide. The same should hold true with the vectors encoding said polynucleotides.

Any cell previously defined as a host cell can be used in these methods.

The invention will be described by way of the following examples which are to be considered as merely illustrative and not limitative of the scope of the invention.

### EXAMPLES

In the following tables we establish a nomenclature to represent sequences that are formed by appending together sequences already listed in the present document. Such sequences are represented, in the following tables, by listing, from the N-terminus to the C-terminus, the sequences, identified by SEQ ID NOs, that constitute them. For example, for a sequence in which aminoacids 1 to 1149 of SEQ ID NO 106 are directly linked to SEQ ID NO 27 (CfaN) which in turn is directly linked to SEQ ID NO 103 (3FT), the nomenclature used would be: SEQ ID NO 106 (from 1 to 1149)-SEQ ID NO 27-SEQ ID NO 103. This specific example should be interpreted as a sequence that starts, from the N-terminus, with amino acids 1 to 1149 of SEQ ID NO 106, which are immediately followed by the polypeptide corresponding to SEQ ID NO 27, directly linked via a peptide bond, which are in turn immediately followed by the polypeptide corresponding to SEQ ID NO 103, also liked via a peptide bond. So that the sequence, would have the following architecture, from N-term to C-term:

[SEQ ID NO 106 (residues from 1 to 1149)]-[SEQ ID NO 27]-[SEQ ID NO 103]

Where the sequences in brackets “[ ]” represent all the amino acids in that sequence, and the “-” represents a peptide bond linking the two sequences in brackets. Residues from x to y of a certain sequence, mean all amino acid residues from position x, to position y, both included. For example, SEQ ID NO 106 (residues from 1 to 1149), means a sequence comprising from residue 1 to residue 1149, both included, from SEQ ID NO 106.

TABLE 5
List of sequences used throughout the examples:

| Construct | ^{a}Sequence | SEQ NO |
|---|---|---|
| ABCA4 | M¹GFVRQIQLLLWKNWTLRKRQKIRFVVELVWPLSLF | 106 |
| | LVLIWLRNANPLYSHHECHFPNKAMPSAGMLPWLQGI | |
| | FCNVNNPCFQSPTPGESPGIVSNYNNSILARVYRDFQ | |
| | ELLMNAPESQHLGRIWTELHILSQFMDTLRTHPERIA | |
| | GRGIRIRDILKDEETLTLFLIKNIGLSDSVVYLLINS | |
| | QVRPEQFAHGVPDLALKDIACSEALLERFIIFSQRRG | |
| | AKTVRYALCSLSQGTLQWIEDTLYANVDFFKLFRVLP | |
| | TLLDSRSQGINLRSWGGILSDMSPRIQEFIHRPSMQD | |
| | LLWVTRPLMQNGGPETFTKLMGILSDLLCGYPEGGGS | |
| | RVLSFNWYEDNNYKAFLGIDSTRKDPIYSYDRRTTSF | |
| | CNALIQSLESNPLTKIAWRAAKPLLMGKILYTPDSPA | |
| | ARRILKNANSTFEELEHVRKLVKAWEEVGPQIWYFFD | |
| | NSTQMNMIRDTLGNPTVKDFLNRQLGEEGITAEAILN | |
| | FLYKGPRESQADDMANFDWRDIFNITDRTLRLVNQYL | |
| | ECLVLDKFESYNDETQLTQRALSLLEENMFWAGVVFP | |
| | DMYPWTSSLPPHVKYKIRMDIDVVEKTNKIKDRYWDS | |
| | GPRADPVEDFRYIWGGFAYLQDMVEQGITRSQVQAEA | |
| | PVGIYLQQMPYPCFVDDSFMIILNRCFPIFMVLAWIY | |
| | SVSMTVKSIVLEKELRLKETLKNQGVSNAVIWCTWFL | |
| | DSFSIMSMSIFLLTIFIMHGRILHYSDPFILFLFLLA | |
| | FSTATIMLCFLLSTFFSKASLAAACSGVIYFTLYLPH | |
| | ILCFAWQDRMTAELKKAVSLLSPVAFGFGTEYLVRFE | |
| | EQGLGLQWSNIGNSPTEGDEFSFLLSMQMMLLDAAVY | |
| | GLLAWYLDQVFPGDYGTPLPWYFLLQESYWLGGEGCS | |
| | TREERALEKTEPLTEETEDPEHPEGIHDSFFEREHPG | |
| | WVPGVCVKNLVKIFEPCGRPAVDRLNITFYENQITAF | |
| | LGHNGAGKTTTLSILTGLLPPTSGTVLVGGRDIETSL | |
| | DAVRQSLGMCPQHNILFHHLTVAEHMLFYAQLKGKSQ | |
| | EEAQLEMEAMLEDTGLHHKRNEEAQDLSGGMQRKLSV | |
| | AIAFVGDAKWILDEPTSGVDPYS¹⁰⁹⁶RRSIWDLLLK | |
| | YRSGRTIIMSTHHMDEADLLGDRIAIIAQGRLY | |
| | C¹¹⁴⁰SGTPLFLKNC¹¹⁵⁰FGTGLYLTLVRKMKNIQS | |
| | QRKGSEGTC¹¹⁷⁷SC¹¹⁷⁹SSKGFS¹¹⁸⁵TTC¹¹⁸⁸P | |
| | AHVDDLTPEQVLDGDVNELMDVVLHHVPEAKLVECIG | |
| | QELIFLLPNKNFKHRAYASLFRELEETLADLGLSSFG | |
| | ISDTPLEEIFLKVTEDSDSGPLFAGGAQQKRENVNPR | |
| | HPCLGPREKAGQTPQDSNVCSPGAPAAHPEGQPPPEP | |
| | ECPGPQLNTGTQLVLQHVQALLVKRFQHTIRSHKDFL | |
| | AQIVLPATFVFLALMLSIVIPPFGEYPALTLHPWIYG | |
| | QQYTFFSMDEPGSEQFTVLADVLLNKPGFGNRCLKEG | |
| | WLPEYPCGNSTPWKTPSVSPNITQLFQKQKWTQVNPS | |
| | PSCRCSTREKLTMLPECPEGAGGLPPPQRTQRSTEIL | |
| | QDLTDRNISDFLVKTYPALIRSSLKSKFWVNEQRYGG | |
| | ISIGGKLPVVPITGEALVGFLSDLGRIMNVSGGPITR | |
| | EASKEIPDFLKHLETEDNIKVWFNNKGWHALVSFLNV | |
| | AHNAILRASLPKDRSPEEYGITVISQPLNLTKEQLSE | |
| | ITVLTTSVDAVVAICVIFSMSFVPASFVLYLIQERVN | |
| | KSKHLQFISGVSPTTYWVTNFLWDIMNYSVSAGLVVG | |
| | IFIGFQKKAYTSPENLPALVALLLLYGWAVIPMMYPA | |
| | SFLFDVPSTAYVALSCANLFIGINSSAITFILELFEN | |
| | NRTLLRFNAVLRKLLIVFPHFCLGRGLIDLALSQAVT | |
| | DVYARFGEEHSANPFHWDLIGKNLFAMVVEGVVYFLL | |
| | TLLVQRHFFLSQWIAEPTKEPIVDEDDDVAEERQRII | |
| | TGGNKTDILRLHELTKIYPGTSSPAVDRLCVGVRPGE | |
| | CFGLLGVNGAGKTTTFKMLTGDTTVTSGDATVAGKSI | |
| | LTNISEVHQNMGYCPQFDAIDELLTGREHLYLYARLR | |
| | GVPAEEIEKVANWSIKSLGLTVYADCLAGTYSGGNKR | |
| | KLSTAIALIGCPPLVLLDEPTTGMDPQARRMLWNVIV | |
| | SIIREGRAVVLTSHSMEECEALCTRLAIMVKGAFRCM | |
| | GTIQHLKSKFGDGYIVTMKIKSPKDDLLPDLNPVEQF | |
| | FQGNFPGSVQRERHYNMLQFQVSSSSLARIFQLLLSH | |
| | KDSLLIEEYSVTQTTLDQVFVNFAKQQTESHDLPLHP | |
| | RAAGASRQAQD²²⁷³ | |
| 3FT | DYKDHDGDYKDHDIDYKDDDDK | 103 |
| ABCA4-1150-Cfa^{N}- | SEQ ID NO 106 (from 1 to 1149)-SEQ ID | 1 |
| 3FT | NO 27-SEQ ID NO 103 | |
| ABCA4-1150-Cfa^{C}- | SEQ ID NO 28-SEQ ID NO 106 (from 1150 | 2 |
| 3FT | to 2273)-SEQ ID NO 103 | |
| ABCA4-1140-Cfa^{N}- | SEQ ID NO 106 (from 1 to 1139)-SEQ ID | 3 |
| 3FT | NO 27-SEQ ID NO 103 | |
| ABCA4-1140- | SEQ ID 29-SEQ ID NO 106 (from 1140 to | 4 |
| Cfa^{Cmut}-3FT | 2273)-SEQ ID 103 | |
| ABCA4-1188-Cfa^{N}- | SEQ ID NO 106 (from 1 to 1187)-SEQ ID | 5 |
| 3FT | NO 27-SEQ ID NO 103 | |
| ABCA4-1188- | SEQ ID NO 29-SEQ ID NO 106 (from 1188 | 6 |
| Cfa^{cmut}-3FT | to 2273)-SEQ ID 103 | |
| ABCA4-3FT | SEQ ID NO 106-SEQ ID NO 103 | 7 |
| ABCA4-1150- | SEQ ID NO 106 (from 1 to 1149)-SEQ ID | 8 |
| Npu^{N}-3FT | NO 32-SEQ ID NO 103 | |
| ABCA4-1150- | SEQ ID NO 33-SEQ ID NO 106 (from 1150 | 9 |
| Npu^{C}-3FT | to 2273)-SEQ ID NO 103 | |
| ABCA4-1140- | SEQ ID NO 106 (from 1 to 1139)-SEQ ID | 10 |
| Npu^{N}-3FT | NO 32-SEQ ID NO 103 | |
| ABCA4-1140- | SEQ ID NO 33-SEQ ID NO (from 1140 to | 11 |
| Npu^{C}-3FT | 2273)-SEQ ID NO 103 | |
| ABCA4-1188- | SEQ ID NO 106 (from 1 to 1187)-SEQ ID | 12 |
| Npu^{N}-3FT | NO 32- SEQ ID NO 103 | |
| ABCA4-1188- | SEQ ID NO 33-SEQ ID NO 106 (from 1188 | 13 |
| Npu^{C}-3FT | to 2273)-SEQ Id NO 103 | |
| EGFP-71-Cfa^{N} | MVSKGEELFTGVVPILVELDGDVNGHKFSVSGEGEGD | 14 |
| | ATYGKLTLKFICTTGKLPVPWPTLVTTLTYGVQCLSY | |
| | DTEILTVEYGFLPIGKIVEERIECTVYTVDKNGFVYT | |
| | QPIAQWHNRGEQEVFEYCLEDGSIIRATKDHKFMTTD | |
| | GQMLPIDEIFERGLDLKQVDGLPGHHHHHHG | |
| EGFP-71-Cfa^{C} | MVKIISRKSLGTQNVYDIGVEKDHNFLLKNGLVASNC | 15 |
| | FSRYPDHMKQHDFFKSAMPEGYVQERTIFFKDDGNYK | |
| | TRAEVKFEGDTLVNRIELKGIDFKEDGNILGHKLEYN | |
| | YNSHNVYIMADKQKNGIKVNFKIRHNIEDGSVQLADH | |
| | YQQNTPIGDGPVLLPDNHYLSTQSALSKDPNEKRDHM | |
| | VLLEFVTAAGITLGMDELYKGHHHHHHG | |
| EGFP | MVSKGEELFTGVVPILVELDGDVNGHKFSVSGEGEGD | 16 |
| | ATYGKLTLKFICTTGKLPVPWPTLVTTLTYGVQCFSR | |
| | YPDHMKQHDFFKSAMPEGYVQERTIFFKDDGNYKTRA | |
| | EVKFEGDTLVNRIELKGIDFKEDGNILGHKLEYNYNS | |
| | HNVYIMADKQKNGIKVNFKIRHNIEDGSVQLADHYQQ | |
| | NTPIGDGPVLLPDNHYLSTQSALSKDPNEKRDHMVLL | |
| | EFVTAAGITLGMDELYKGHHHHHHG | |
| EGFP-71-Npu^{N} | MVSKGEELFTGVVPILVELDGDVNGHKFSVSGEGEGD | 17 |
| | ATYGKLTLKFICTTGKLPVPWPTLVTTLTYGVQCLSY | |
| | ETEILTVEYGLLPIGKIVEKRIECTVYSVDNNGNIYT | |
| | QPVAQWHDRGEQEVFEYCLEDGSLIRATKDHKFMTVD | |
| | GQMLPIDEIFERELDLMRVDNLPNGHHHHHHG | |
| EGFP-71-Npu^{C} | MIKIATRKYLGKQNVYDIGVERDHNFALKNGFIASNC | 18 |
| | FSRYPDHMKQHDFFKSAMPEGYVQERTIFFKDDGNYK | |
| | TRAEVKFEGDTLVNRIELKGIDFKEDGNILGHKLEYN | |
| | YNSHNVYIMADKQKNGIKVNFKIRHNIEDGSVQLADH | |
| | YQQNTPIGDGPVLLPDNHYLSTQSALSKDPNEKRDHM | |
| | VLLEFVTAAGITLGMDELYKGHHHHHHG | |
| ABCA4-1150-Cfa^{N}- | SEQ ID NO 106 (from 1 to 1149)-SEQ ID | 19 |
| 3FT-SopE | NO 27-SEQ ID NO 103-SEQ ID NO 48 | |
| ABCA4-1150-Cfa^{C}- | SEQ ID NO 28-SEQ ID NO 106 (from 1150 | 20 |
| 3FT-SopE | to 2273)-SEQ ID NO 103-SEQ ID NO 48 | |
| ABCA4-1140-Cfa^{N}- | SEQ ID NO 106 (from 1 to 1139)-SEQ ID | 21 |
| 3FT-SopE | NO 27-SEQ Id NO 103-SEQ ID NO 48 | |
| ABCA4-1140- | SEQ ID NO 29SEQ ID NO 106 (from 1140 | 22 |
| Cfa^{Cmut}-3FT-SopE | to 2273)-SEQ ID NO 103-SEQ ID NO 48 | |
| EGFP-71-Cfa^{N}- | SEQ ID NO 14-SEQ ID NO 48 | 23 |
| SopE | | |
| EGFP-71-Cfa^{C}- | MTKITLSPQNFRIQKQETTLLKEKSTEKNSLAKSILA | 24 |
| SopE | VKNHFIELRSKLSERFISHKNTESSATHFHRGSASEG | |
| | RAVLTNKVVKDFMLQTLNDIDIRGSAVKIISRKSLGT | |
| | QNVYDIGVEKDHNFLLKNGLVASNCFSRYPDHMKQHD | |
| | FFKSAMPEGYVQERTIFFKDDGNYKTRAEVKFEGDTL | |
| | VNRIELKGIDFKEDGNILGHKLEYNYNSHNVYIMADK | |
| | QKNGIKVNFKIRHNIEDGSVQLADHYQQNTPIGDGPV | |
| | LLPDNHYLSTQSALSKDPNEKRDHMVLLEFVTAAGIT | |
| | LGMDELYKGHHHHHHG | |
| EGFP-71-Cfa^{N}-DD1 | SEQ ID NO 14-SEQ ID NO 43 | 25 |
| EGFP-71-Cfa^{C}-DD1 | MRISFGSPPPMAGGVKIISRKSLGTQNVYDIGVEKDH | 26 |
| | NFLLKNGLVASNCFSRYPDHMKQHDFFKSAMPEGYVQ | |
| | ERTIFFKDDGNYKTRAEVKFEGDTLVNRIELKGIDFK | |
| | EDGNILGHKLEYNYNSHNVYIMADKQKNGIKVNFKIR | |
| | HNIEDGSVQLADHYQQNTPIGDGPVLLPDNHYLSTQS | |
| | ALSKDPNEKRDHMVLLEFVTAAGITLGMDELYKGHHH | |
| | HHHG | |
| ABCA4^{N}-1150 | SEQ ID NO 106 (from 1 to 1149) | 60 |
| ABCA4^{C}-1150 | SEQ ID NO 106 (from 1150 to 2273) | 61 |
| ABCA4^{N}-1140 | SEQ ID NO 106 (from 1 to 1139) | 62 |
| ABCA4^{C}-1140 | SEQ ID NO 106 (from 1140 to 2273) | 63 |
| ABCA4^{N}-1188 | SEQ IS NO 106 (from 1 to 1187) | 64 |
| ABCA4^{C}-1188 | SEQ ID NO 106 (from 1188 to 2273) | 65 |
| ABCA4-1150-Cfa^{N} | SEQ ID NO 60-SEQ ID NO 27 | 66 |
| ABCA4-1150-Cfa^{C} | SEQ ID NO 28-SEQ ID NO 61 | 67 |
| ABCA4-1140-Cfa^{N} | SEQ ID NO 62-SEQ ID NO 27 | 68 |
| ABCA4-1140- | SEQ ID NO 29-SEQ ID NO 63 | 69 |
| Cfa^{cmut} | | |
| ABCA4-1188-Cfa^{N} | SEQ ID NO 64-SEQ ID NO 27 | 70 |
| ABCA4-1188- | SEQ ID NO 29-SEQ ID NO 65 | 71 |
| Cfa^{cmut} | | |
| ABCA4-1150-Cfa^{N}- | SEQ ID NO 66-SEQ ID NO 103-SEQ ID NO | 72 |
| SopE | 48 | |
| ABCA4-1150-Cfa^{C}- | SEQ ID NO 48-SEQ ID NO 67- SEQ ID NO | 73 |
| SopE | 103 | |
| ABCA4-1140-Cfa^{N}- | SEQ ID NO 68-SEQ ID NO 103-SEQ ID NO | 74 |
| SopE | 48 | |
| ABCA4-1140- | SEQ ID NO 48-SEQ ID NO 69-SEQ ID NO | 75 |
| Cfa^{Cmut}-SopE | 103 | |
| ABCA4-1150-Cfa^{N}- | SEQ ID NO 66-SEQ ID NO 43, where X is | 76 |
| DD1 | Gly. | |
| ABCA4-1150-Cfa^{C}- | SEQ ID NO 67-SEQ ID NO 43, where X is | 77 |
| DD1 | Gly. | |
| ABCA4-1140-Cfa^{N}- | SEQ ID NO 68-SEQ ID NO 43, where X is | 78 |
| DD1 | Gly. | |
| ABCA4-1140- | SEQ ID NO 69-SEQ ID NO 43, where X is | 79 |
| Cfa^{Cmut}-DD1 | Gly. | |

Materials and Methods

Materials:

Oligonucleotides were purchased from Eurofins genomics. Synthetic genes were purchased from GENEWIZ. Pfu Ultra fusion polymerase for cloning and all restriction enzymes were purchased from Thermofisher Scientific. High-competency cells used for cloning were generated from XL10-Gold chemically competent E. coli. HEK293T cells were purchased from ATCC. DNA purification kits were purchased from Qiagen. All plasmids were sequenced by Macrogen. Luria Bertani (LB) media, and all buffering salts were purchased from Thermofisher Scientific. Coomassie brilliant blue, MG-132 proteosome inhibitor, phenylmethane sulfonyl fluoride, iodoacetamide, NH4HCO3, DTT, formic acid, fetal bovine serum and asolectin from soybean were purchased from Sigma-Aldrich. Acetonitrile (ACN) was purchased from Carlo-Erba. EDTA-free complete protease inhibitors were purchased from Roche. Lipofectamine 2000 transfection reagent, DMEM high glucose GlutaMAX supplement, RPMI 1640 medium GlutaMAX supplement, RIPA lysis and extraction buffer, BCA protein assay kit, MES-SDS running buffer, pre-stained protein ladder and SDS-PAGE (Bis-tris and Tris-acetate gels) were purchased from Thermofisher Scientific. The primary anti-6×His tag mouse monoclonal antibody, anti-flag tag mouse monoclonal antibody, anti-ABCA4 rabbit polyclonal antibody and anti-tubulin rabbit polyclonal antibody were purchased from Invitrogen. The secondary goat anti-mouse IgG (H+L) highly cross-adsorbed antibody alexa fluor plus 680 and goat anti-rabbit IgG (H+L) secondary antibody dylight 800 4×PEG were purchased from Invitrogen. Dodecyl maltoside (D310) and cholesteryl hemisuccinate (CH210) solution were purchased from Anatrace. Trypsin was purchased from Promega.

Equipment:

Electrospray ionization mass spectrometric analysis (ESI-MS) was carried out on a no Acquity liquid chromatographer (Waters) coupled to a LTQ-Orbitrap Velos (Thermo Scientific) mass spectrometer. Gels and Western-blots were imaged with a LI-COR Odyssey Infrared Imager. Cell lysis was carried out using a SFX550 Branson sonifier. FACS measurements were performed on a Gallios Beckman Coulter.

Cloning of Recombinant DNA

Synthetic genes to prepare constructs ABCA4-1150-CfaN (SEQ ID NO 1) and ABCA4-1150-CfaC (SEQ ID NO 2) were purchased and introduced into pEGFP-N1 expression vectors using Kpnl and Notl restriction enzymes. Synthetic genes for NpuN and NpuC were purchased and introduced into ABCA4-1150-CfaN and ABCA4-1150-CfaC by restriction enzyme free cloning to obtain the constructs ABCA4-1150-NpuN (SEQ ID NO 8) and ABCA4-1150-NpuC (SEQ ID NO 9).

Constructs ABCA4-3FT (SEQ ID NO 7), ABCA4-1140-CfaN (SEQ ID NO 3), ABCA4-1140-CfaCmut (SEQ ID NO 4), ABCA4-1188-CfaN (SEQ ID NO 5), ABCA4-1188-CfaCmut (SEQ ID NO 6) ABCA4-1179-CfaN (SEQ ID NO 93) and ABCA4-1179-CfaCmut (SEQ ID NO 94) were prepared by restriction enzyme free cloning. The mutations at the CfaCmut were introduced using inverse PCR with Pfu Ultra II HF Polymerase. ABCA4-1140-NpuN (SEQ ID NO 10), ABCA4-1140-NpuC (SEQ ID NO 11), ABCA4-1188-NpuN (SEQ ID NO 12) and ABCA4-1188-NpuC (SEQ ID NO 13) were prepared by restriction enzyme free cloning.

Construct EGFP-71-CfaN (SEQ ID NO 14), EGFP-71-CfaC (SEQ ID NO 15), EGFP (SEQ ID NO 16), EGFP-71-NpuN (SEQ ID NO 17) and EGFP-71-NpuC (SEQ ID NO 18) were prepared by restriction enzyme free cloning.

Synthetic gene for SopE was purchased and introduced into ABCA4-1150-CfaN, ABCA4-1150-CfaC, ABCA4-1140-CfaN, ABCA4-1140-CfaC, EGFP-71-CfaN and EGFP-71-CfaC by restriction enzyme free cloning to obtain the constructs ABCA4-1150-CfaN-SopE (SEQ ID NO 19), ABCA4-1150-CfaC-SopE (SEQ ID NO 20), ABCA4-1140-CfaN-SopE (SEQ ID NO 21), ABCA4-1140-CfaC-SopE (SEQ ID NO 22), EGFP-71-CfaN-SopE (SEQ ID NO 23) and EGFP-71-CfaC-SopE (SEQ ID NO 24). The gene for DD1 was prepared by overlapped extension PCR and introduced into EGFP-71-CfaN and EGFP-71-CfaC by restriction enzyme free cloning to obtain the constructs EGFP-71-CfaN-DD1 (SEQ ID NO 25) and EGFP-71-CfaC-DD1 (SEQ ID NO 26). The identity of all recombinant plasmids was confirmed through sequencing and the corresponding protein sequences are reported in Table 1. Constructs with degrons, were generated analogously by cloning the different elements including the split protein gene, the split intein and the degron into expression plasmids.

AAV Production.

AAVs encoding N-fragments of the invention encoding EGFP or ABCA4 fragments with the CfaN intein were produced using standard triple transfection strategies. rAAV8 vectors with wild-type AAV2 ITRs were produced by polyethylenimine (PEI) mediated co-transfection of plasmids pAAV-Rep2-Cap8, pHelper and AAV-transgene-plasmids in HEK293 cells.

Cells and medium were harvested 48 hr after transfection. Virus were released from the cells following treatment with 0.1% Triton and AAV particles from culture medium was obtained by PEG-precipitation. Purification of crude lysate and removal of empty capsid was performed by iodixanol gradients according to the method of Zolotukhin and colleagues [Zolotukhin S, Byrne B J, Mason E, Zolotukhin I, Potter M, Chestnut K, Summerford C, Samulski, R J, Mucyczka N. (1999) Recombinant adeno-associated virus purification using novel methods improves infectious titer and yield. Gene Ther; 6(6): 973-85.].

The purified batches were concentrated and formulated using Vivaspin 20 Centrifugal Concentrator 100.000 MWCO (Sartorius, Cat.No. VS 0641) to a final concentration of 1×10¹³ vg/ml, as determined by quantitative polymerase chain reaction (qPCR). The formulation buffer used is characterized by BSS (Alcon)+Pluronic.

After concentration, the viral batches were filtered using Acrodisc® syringe filters (Acrodisc PP, PES, 0.2 μM 1 cm2) and stored at −80° C. Viral titers in terms of genome copies per milliliter were determined by qPCR using ITR specific PCR-primers. Moreover, capsid titers were obtained using AAV8 ELISA (Progen) following manufacturer's instructions. Protein present in the final product and corresponding to 1×10¹⁰ vg was separated on a 10% SDS PAGE using Pierce Silver stain kit (Cat #24612) for visualization of proteins. VP1, VP2 and VP3 proteins in the correct stoichiometry of 1:1:10 are detectable indicating a purity of the AAV preparation of >95%.

Transfection of Intein Plasmids in HEK293 Cells:

HEK293T cells were maintained in DMEM with 10% FBS and antibiotics at 37° C. in a 6% CO2 atmosphere. Cells were co-transfected at around 80% confluence using Lipofectamine 2000 and 1.25 μg of each plasmid in 6-well plate format. For the experiments where the plasmid encoded the full-length gene was used, a scramble plasmid was co-transfected with the full-length plasmid to achieve the same amount of DNA transfected when two intein plasmid were used. Cells were harvested after 48 h post-transfection and EGFP was analyzed by Western blot.

Transfection of Degron-Intein Plasmids in HEK293 Cells:

Cells were co-transfected at around 80% confluence using Lipofectamine 2000 and 1.25 μg of each plasmid in 6-well plate format. Cells transfected with intein-degron plasmids were harvested after 48 h post-transfection and analyzed by Western blot. For time course experiments, cells were harvested after 24 and 48 h post-transfection and analyzed by Western blot.

Proteosome inhibitor experiments were performed using MG-132 inhibitor. Cells were co-transfected and 24 h post-transfection DMSO or MG-132 (dissolved in DMSO) was added to a final concentration of 50 [symbol] M. Cells were harvested after 30 min, 3, 6 and 24 hours and analyzed by Western blot.

Western Blot Analysis:

EGFP transfected cells (HEK293 and WERI-RB1 cells) were lysed in RIPA buffer supplemented with protease inhibitors and 1 mM phenylmethylsulfonyl. After lysis, samples were quantified by BCA protein assay kit. Samples with 10 μg of total protein were denatured at 95° C. for 10 minutes in 1× Laemmli sample buffer. Lysates were separated by 12% Bis-tris SDS-PAGE gels for 40 min at 165V. The antibodies used for immuno-blotting were anti-6×His tag to detect the EGFP and anti-β-tubulin as loading control. The quantification of EGFP bands detected by Western blot was performed using LI-COR Odyssey Infrared Imager.

ABCA4 transfected cells (HEK293) were lysed in dodecyl maltoside (D310) and cholesteryl hemisuccinate (CH210) solution in PBS (1:1) supplemented with protease inhibitors and 1 mM phenylmethylsulfonyl. After lysis, ABCA4 samples were quantified by BCA protein assay kit. Samples with 25 μg of total protein were denatured at 37° C. for 15 minutes in 1×Laemmli sample buffer containing 2.5 mg/ml of asolectin. Lysates were separated by 3-8% Tris-acetate SDS-PAGE gels for 1.5 h at 150V. The antibodies used for immuno-blotting were either anti-flag tag or anti-ABCA4 to detect the ABCA4 protein and anti-8-tubulin as loading control. The quantification of ABCA4 bands detected by Western blot was performed using LI-COR Odyssey Infrared Imager.

Fluorescence-Activated Cell Sorting Measurement:

HEK293T cells were co-transfected with 2 μg of each EGFP-intein plasmid and as a negative control HEK293T cells were grown but they were not transfected. For the experiments where the plasmid encoded the full-length gene was used, a scramble plasmid was co-transfected with the full-length plasmid to achieve the same amount of DNA transfected when two intein plasmid were used. Cells were analyzed by flow cytometry after 48 h post-transfection. Transfected and untransfected cells were respectively resuspended in FACS analysis buffer (PBS, 2% FSA, 2 mM EDTA). The percentages of EGFP+ cells were assessed by comparing the different transfected cells to un-transfected cells in a Beckman Coulter Gallios flow cytometer using the Kaluza flow cytometry analysis software. Dapi staining was used to detect the number of dead cells.

Mass Spectrometry Analysis:

The gel bands were washed with ammonium bicarbonate (50 mM NH4HCO3) and acetonitrile (ACN). The samples were reduced with 20 mM DTT for 60 min at 60 [symbol] C and alkylated with 55 mM iodoacetamide at 25° C. for 30 min in the dark. Afterwards, the samples were digested double digested for 2 h and overnight at 37° C. with trypsin (sequence grade modified Trypsin). Finally, the resulting peptide mixtures were extracted from the gel matrix with 5% formic acid (FA) in 50% ACN, and 100% ACN, and dried-down in a SpeedVac vacuum system. The resulting peptide mixtures were cleaned-up with a C18 tip (PolyLC Inc.) as per manufacturer's protocol. Finally, the cleaned-up peptide solutions were dried-down. The tryptic digest mixture was resuspended in 1% FA solution and for each sample, an aliquot was injected for chromatographic separation. Peptides were trapped on a Symmetry C18 trap column (Sum 180 μm×20 mm; Waters) and were separated using a C18 reverse phase capillary column (ACQUITY UPLC M-Class Peptide BEH column; 130 Å, 1.7 μm, 75 μm×250 mm, Waters). The gradient used for the elution of the peptides was 1 to 40% B in 25 minutes, followed by gradient from 40% to 60% in 5 min (A: 0.1% FA; B: 100% ACN, 0.1% FA), with a 250 nL/min flow rate. Eluted peptides were subjected to electrospray ionization in an emitter needle (PicoTip™, New Objective) with an applied voltage of 2000V. Peptide masses (m/z 300-1700) were analyzed in data dependent mode where a full Scan MS was acquired in the Orbitrap with a resolution of 60,000 FWHM at 400 m/z. Up to the 15th most abundant peptides (minimum intensity of 500 counts) were selected from each MS scan and then fragmented in the linear ion trap using CID (38% normalized collision energy) with helium as the collision gas. Database search was performed with Sequest HT search engine using Thermo Proteome Discover.

### In Vivo Experiments

Experiments were performed with 4 weeks-old wild-type animals, strain 1295v. Mice were injected subretinally with test samples.

Subretinal injection were performed following pupil dilation, using a 33G needle on a 5 ml Hamilton syringe. 4-week post injection EGFP expression eye fundus evaluation was achieved using a Canon UVI retinal camera connected to a digital imaging system. Retinal structure and quantification of Outer nuclear layer (ONL) was evaluated by SD-OCT (Optical Coherence Tomography) using a Spectralis imaging system (Heidelberg Engineering Inc.). After imaging, animals were euthanized, and eyes enucleated for further analysis by immune histochemistry and western blot. Additional experiments were performed in which vectors were injected in the inner ear of 6 weeks C57BL/6 mice.

### Results and Discussion

Recently several new inteins have been engineered based on consensus design ((Stevens et al., 2016; Stevens, Sekar, Gramespacher, Cowburn, & Muir, 2018)) and shown to have superior properties than naturally occurring inteins. One of these inteins, termed Cfa, was generated by consensus design from an alignment of DnaE inteins and shown to have superior properties to some of the best performing inteins of the DnaE family such as Npu. Cfa was reported to have faster kinetics, higher expression levels and high tolerance to extreme conditions such as high temperature and concentration of denaturing agents. Interestingly, Cfa variants with degrees of homology from 90% or higher display similar properties. The same consensus design strategy was applied to the TerL-AceL intein family, which resulted in a consensus sequence termed Cat, which also shows improved properties relative to the rest of the members of the TerL-AceL family.

In order to demonstrate that consensus inteins provided a benefit for the reconstitution of proteins via protein trans-splicing for gene therapy applications, a direct comparison was performed. Comparisons were performed by transfecting cells with purified plasmids.

Initial experiments were performed using EGFP as a reporter gene. Briefly, EGFP was split at position 71 and the N-terminal fragment (residues 1-70, EGFPN) recombinantly fused to N-inteins, and the C-terminal fragment (residues 71-239, EGFPC) to the C-inteins. The following four constructs were generated: EGFPN-CfaN, EGFPN-NpuN, CfaC-EGFPC and NpuC-EGFPC, in order to compare the reconstitution efficiency of the Cfa consensus sequence with Npu. Cultured HEK293 cells were co-transfected with equimolar amounts of plasmids encoding for the N and C-terminal fragments and splicing efficiency monitored by fluorescence microscopy, flow cytometry analysis and Western blotting.

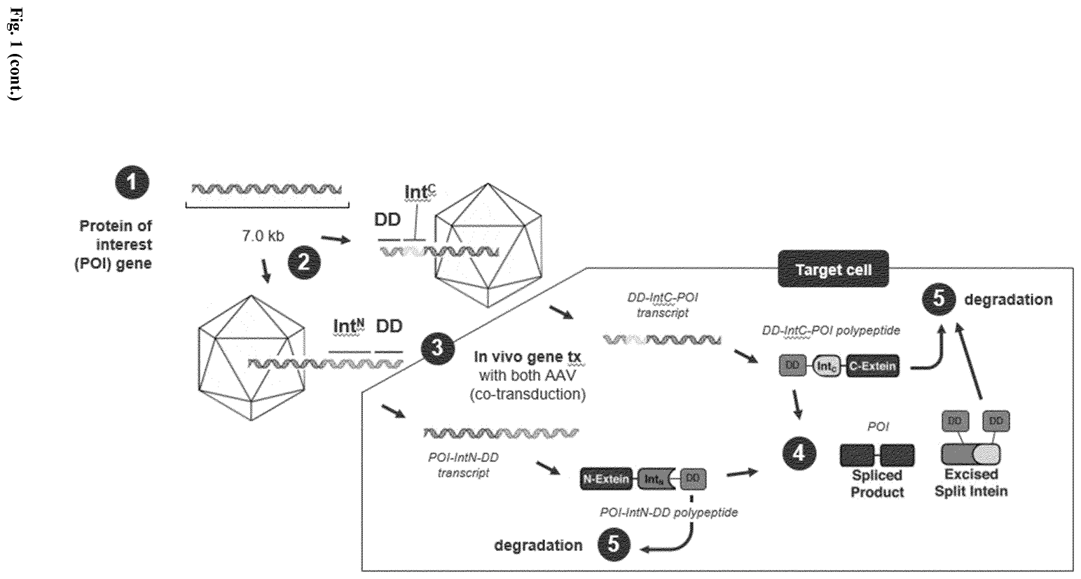

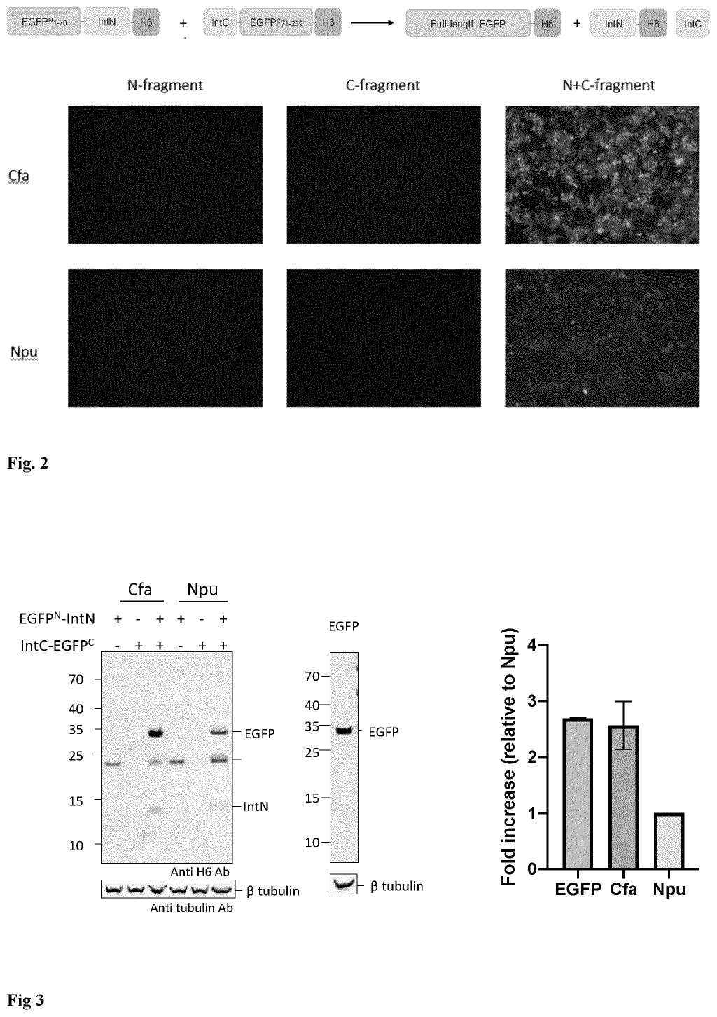

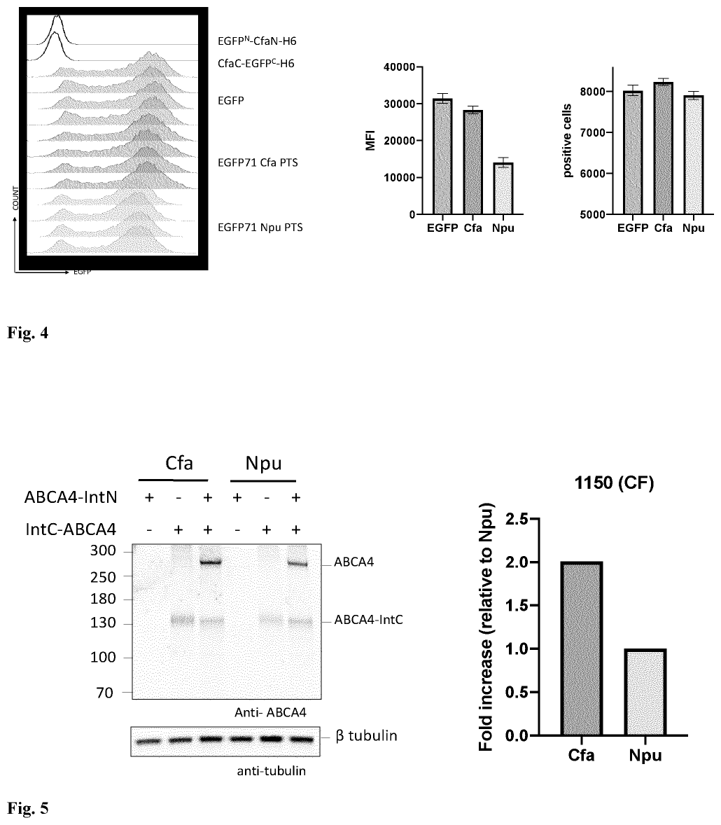

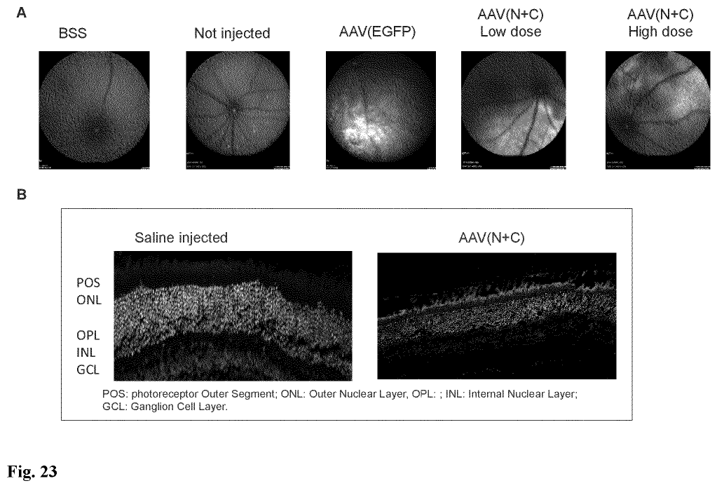

Our results, see FIGS. 2, 3 and 4, show that the consensus Cfa intein provides a 2.5 fold increase in EGFP reconstitution compared with the Npu intein, indicating that inteins obtained by consensus design provided higher yields of target protein upon co-transfection. It is important to point out that although Cfa was known to split at faster rates than Npu in vitro, that its N-terminal fragments expressed better when individually transformed or transfected in cells, it had not been shown if this resulted in a benefit when both fragments were co-transfected in the same cell. Through these experiments we demonstrate, that when both IntN and IntC fragments are expressed in the same cell, the use of Cfa versus previously known ultra-fast split intein, such as Npu, provides a clear benefit in the reconstitution yield of the target protein. The genes encoding for the N- and C-fragments of EGFP and the N and C-inteins were also incorporated into recombinant AAVs for their in vivo delivery. Results demonstrated that the Cfa intein mediated protein splicing resulted in EGFP reconstitution in several organs, including the retina and the inner ear (FIG. 23).

In order to confirm that this positive feature of the Cfa consensus sequence was observed in other proteins we repeated the experiment using the ABCA4 protein. ABCA4 is a large protein, which is mutated in Stargardt disease, and whose reconstitution has been proposed as a viable strategy to treat the disease. Several approaches based on AAV gene therapy are currently being explored to reconstitute it. Due to its large size ABACA4 cannot be encapsulated into a single AAV, and so different strategies have been proposed to be able to deliver ABCA4 in two fragments, to be reconstituted inside the target cells. In order to confirm that engineered consensus sequences such as Cfa provided a benefit over naturally occurring inteins such as Npu, we split ABCA4 at position 1150 and cloned the N-inteins and C-inteins to the N- and C-terminal fragments of the protein, respectively.

ABCA4^{N}(1-1149)-IntN and IntC-ABCA4^{C}(1150-2273) with the Cfa, or Npu inteins were cloned into expression plasmids and co-transfected into HEK293 cells. Cells were lysed and protein reconstitution yields determined by Western Blot. Full-length ABCA4 was also co-transfected to serve as control.

As observed with EGFP we observed higher reconstitution yields with Cfa than with Npu (see FIG. 5).

A variant of Cfa, which contains specific mutations in the CfaC fragment, termed CfaCmut ((Stevens et al., 2017)), has also been recently reported to add to the Cfa benefits. Particularly the CfaCmut variant has been shown to be able to efficiently splice protein fragments even if the +2 position of the C-extein does not contain the native Phe residue. The +2 position, as defined above, refers to the position +2, relative to the last amino acid of the IntC. When ABCA4 is split at position 1150, that leaves a Phe in the C-extein +2 position, which is an optimal residue for Npu, as well as Cfa.

Despite ABCA4 can be reconstituted by splitting it at position 1150, this might not be the best possible position for therapeutic purposes. In order to test our ability to reconstitute ABCA4 by splitting it at different sites than 1150, we used the CfaCmut, in order not to be limited to positions that would render a Phe at the position +2, relative to the intein.

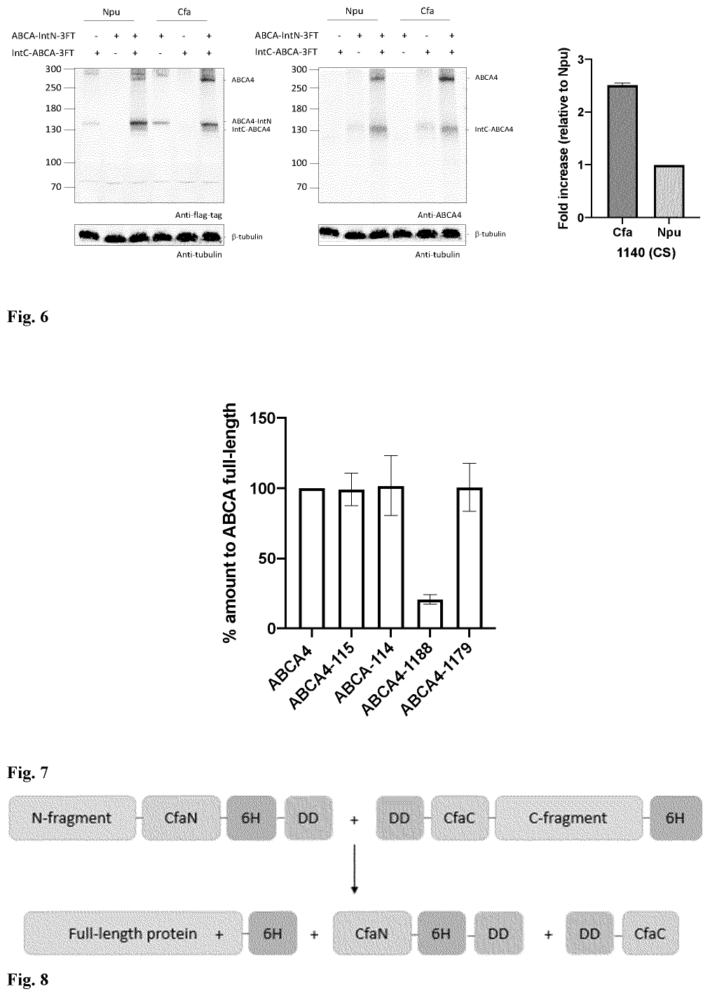

Several ABCA4 constructs were cloned in which the ABCA4 protein was split at sites that did not contain a Phe on the position +2 of the split site. Two of the additional sites that were tested were position 1140, which corresponds to a Ser at the +2 site, and position 1188 which corresponds to a Pro at the +2 site. Constructs were cloned and tested as shown above. Specifically, ABCA4(1-1139)-IntN, IntC-ABCA4(1140-2273) constructs were used to evaluate splicing ABCA4 at position 1140. And ABCA4(1-1187)-IntN and IntC-ABCA4(1188-2273) to evaluate splitting ABCA4 at position 1188. Sites were selected based on the topological structure of ABCA4 and taking into consideration the presence of folded domains. Sites were selected outside of well-defined folded domains in the intracellular side of ABCA4. Different sites were tested, and it was observed for all of them that Cfa and Cfa variants provided higher reconstitution yields than Npu and that the efficiency was also different depending on the split site. (FIGS. 5, 6 and 7). We also demonstrated that the approach allowed reconstitution of ABCA4 split at position 1179 and 1177. Analogous constructs to the ones defined above for 1140 and 1188 were cloned and used to test efficiency of splicing at positions 1179 and 1177, results at position 1179 are shown in FIG. 21.

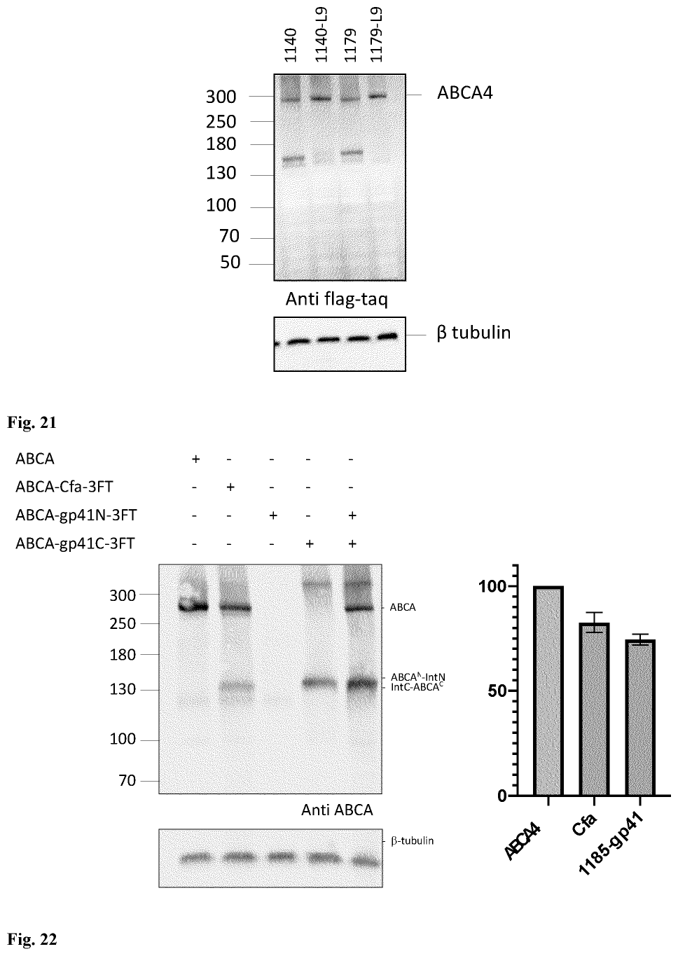

We also demonstrated that other efficient inteins such as gp41 could be used to reconstitute ABCA4 from split fragments. ABCA4 constructs split at positions 1185 and 1095 were designed, and the corresponding fusions to the N- and C-terminal inteins were cloned and their splicing activity analyzed as described above. Analysis by Western blot of the PTS reaction at the split position 1096 and the gp41 intein is shown in FIG. 22.

Table 7 below shows the sequences of the ABCA4 fragments obtained when the protein is split at different sites (1177, 1179, 1096 and 1185) as well as the corresponding ABCA4-InteinN or InteinC-ABCA4 fusions.

Besides reconstitution yield another of the major limitations of the use of split inteins in gene therapy is the presence of unreacted starting materials and/or inteins. To prevent the accumulation of undesired starting materials and to eliminate the excised intein fragments we added a degron to the constructs with the general architecture shown in FIG. 8.

We have identified several degrons that could be used in combination with inteins to develop gene therapy approaches for ABCA4, which could be translated to diseases caused by mutations on other large genes, as well as to reconstitute proteins to treat those diseases, for example to reconstitute CRISPR/Cas9 systems.

In order to identify suitable degron-intein combinations EGFP-Int constructs were cloned including the selected degron. The degron was cloned at the C-terminus of the N-intein, and the N-terminus of the C-intein. A His6 tag was included as a linker between the two elements for detection purposes. As a proof of principle, the SopE destabilizing domain (1-100) was used, as well as a degron consisting of the peptide sequence DD1 (see Table 3)

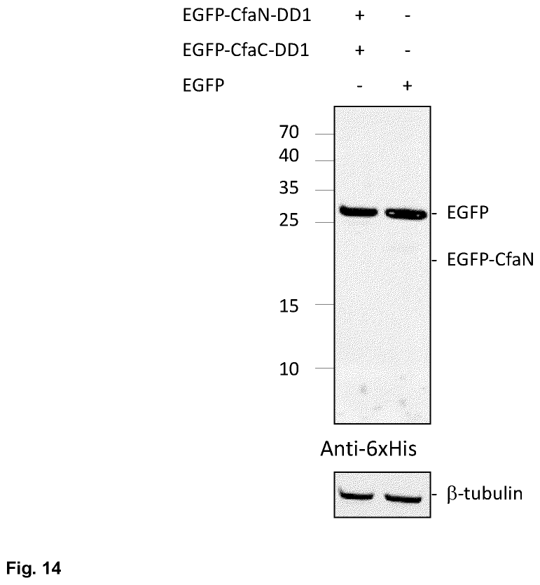

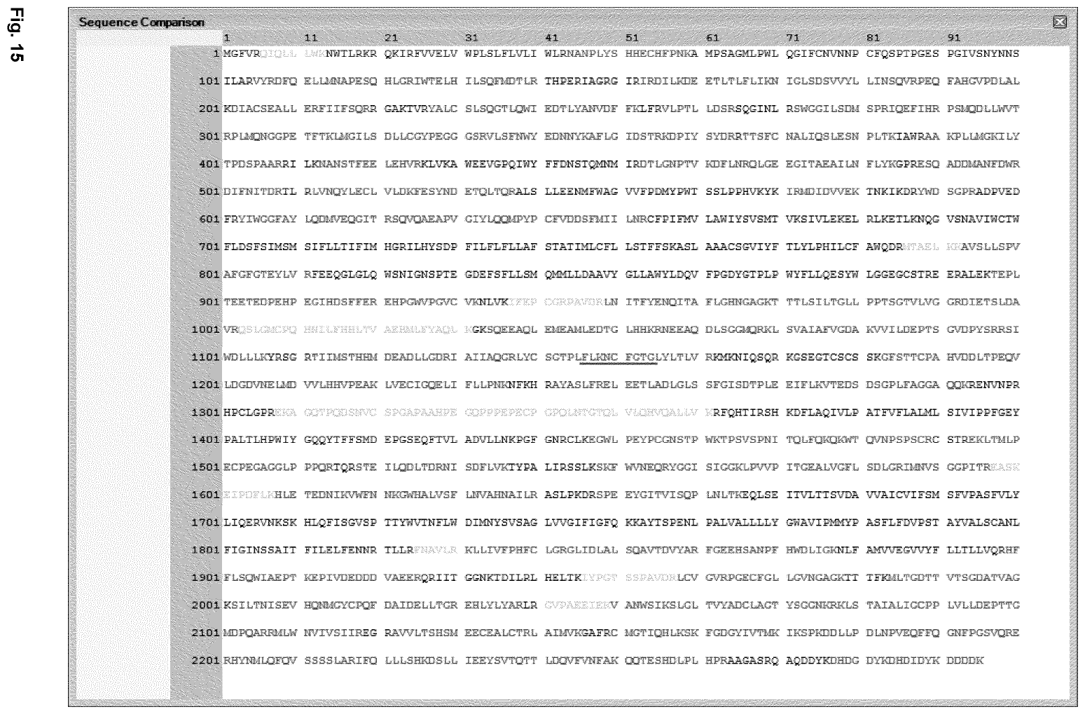

Results show that the inclusion of the degron indeed removes any detectable amounts of starting materials and inteins (see FIG. 9, FIG. 14). Importantly, and unexpectedly, it was demonstrated that including both degrons simultaneously allowed the removal of any unreacted starting materials without any loss of yield on the protein splicing reaction.

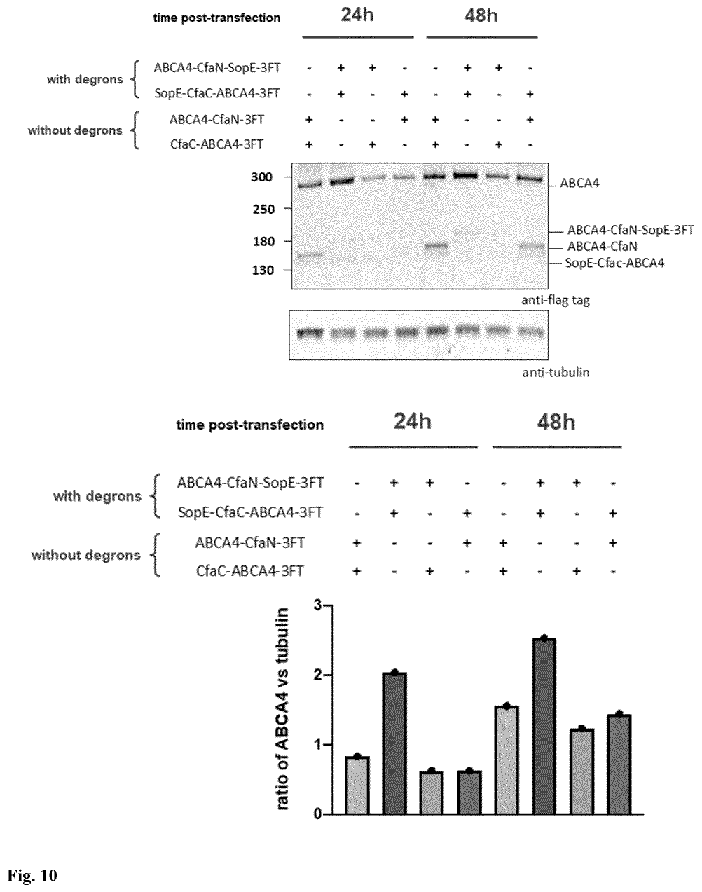

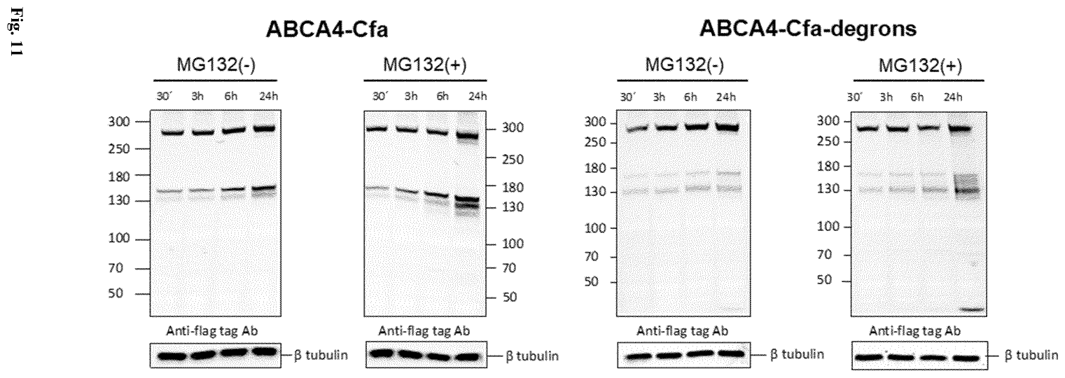

Interestingly, when the degron strategy was applied to the large gene ABCA 4 we observed a similar result but also an increase on the levels of spliced ABCA4 product, indicating an unexpected synergistic effect between consensus inteins and degrons (see FIG. 10).

The effect on these results of inhibitors of the proteosome was also studied (FIG. 11). We showed that proteosome inhibitors reduced the effect of the degron, thus confirming that SopE degradation was proteosomally mediated. Interestingly, we observed that combining inteins with degrons we were able not only to eliminate the presence of starting materials but also to maintain, or even increase the level of reconstitution of ABCA4. (FIG. 10).

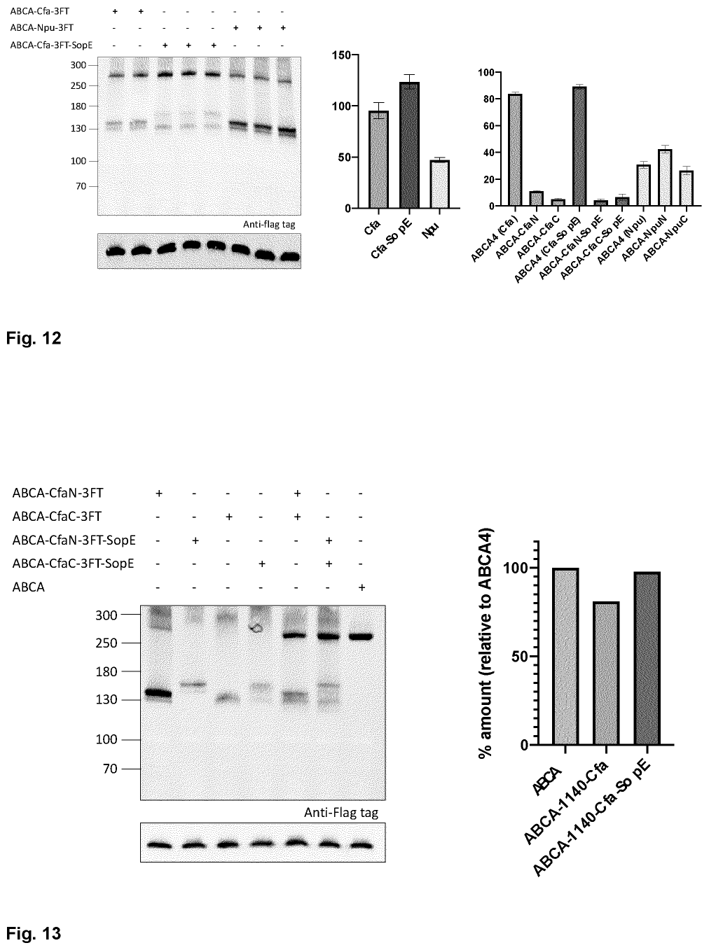

We also performed experiments to study the effect of combining consensus intein Cfa with degrons and compared it with the absence of degrons or the use of other inteins. Interestingly, we observed (FIG. 12) that the use of Cfa with degrons very significantly reduced the levels of starting materials and also increased the amount of product. We also confirmed that this effect was also observed at position 1140 of ABCA4 (FIG. 13 and FIG. 21) and 1179 (FIG. 21). Importantly, we showed that the effect observed combining Cfa (and Cfamut) inteins with several different degrons, which shared certain features, was maintained (see Example 3).

Based on the results obtained with ABCA4, other diseases, genes and proteins in which this approach could be applied include the ones shown in table 1.

### Example 2. Three-Piece Ligation Strategy Using Orthogonal Split Intein Pairs

Material and Methods:

Transfection of Three-Piece Plasmids in HEK293 Cells:

Cells were co-transfected at around 80% confluence using Lipofectamine 2000 and 1.25 μg of each plasmid in 6-well plate format. Cells transfected with three-piece plasmids were harvested after 48 h post-transfection and analyzed by Western blot. As a control, cells were co-transfected with two plasmids (POIN-CfaN+CfaC-POIM-CatN, CfaC-POIM-CatN+CatC-POIC and POIN-CfaN+CatC-POIC).

Western Blot Analysis:

Three-piece transfected cells (HEK293) were lysed in RIPA buffer supplemented with protease inhibitors and 1 mM phenylmethylsulfonyl. After lysis, samples were quantified by BCA protein assay kit. Samples with 10 μg of total protein were denatured at 95° C. for 10 minutes in 1× Laemmli sample buffer. Lysates were separated by 12% Bis-tris SDS-PAGE gels for 40 min at 165V. The antibodies used for immuno-blotting were anti-flag tag to detect the POI and anti-β-tubulin as loading control.

Results

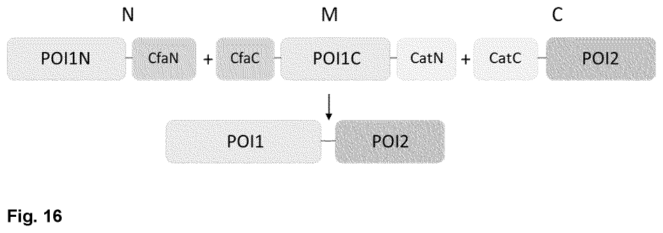

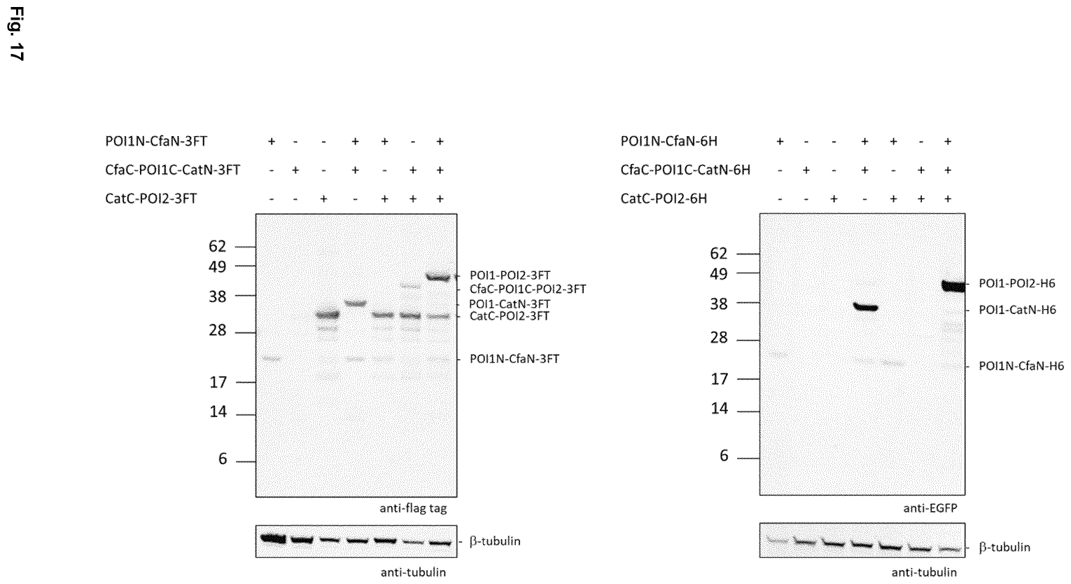

We showed combining ultrafast, consensus inteins with degrons is a viable strategy to reconstitute large proteins, while reducing the levels of starting materials and excised inteins. This strategy could be used in gene therapy to reconstitute large proteins in gene replacement strategies, as well as to generate therapeutic agents in vivo, when combined with suitable delivery vectors, such as the ones disclosed here. However, for even larger proteins this strategy might not be sufficient. For example, for proteins whose coding region is larger than 7-8 kb, splitting their coding genes in two fragments would not be sufficient to yield fragments that can be encapsulated in AAV vectors. For such proteins, the coding gene may need to be split into three pieces. In order to assemble a protein from three separate fragments using inteins, orthogonal intein pairs would be required. To achieve maximum reconstitution yields, highly efficient, orthogonal inteins would be required. We decided to use Cfa in combination with Cat inteins. These two inteins have been obtained by consensus design and share some common features, including high expression yields, thermostability, tolerance to chaotropic agents and fast splicing kinetics. We designed a series of constructs following the architecture described in FIG. 16: POIN-CfaN, CfaC-POIM-CatN and CatC-POIC. Alternatively, the positions of the Cfa and Cat inteins could be swapped, to generate constructs with the following architecture: POIN-CatN, CatC-POIM-CfaN and CfaC-POIC

We tested their orthogonality and demonstrated that both inteins were indeed orthogonal and they did not react with each other, that is that the N-fragment of the Cfa, or Cat intein, only reacted with their respective cognate pair. When the three fragments were co-transfected in equimolar amounts the main product that was detected was the one arising from the assembly of the three fragments into the full-length desired product. Importantly, we only detected low levels of unreacted materials and we did not detect any intermediate product, that is products arising from the reaction of only two of the fragments. This result indicates that the use of highly active pairs of orthogonal consensuses inteins is a suitable strategy to assemble large proteins from three individual fragments.

### Example 3

To practice the present invention, several degrons were tested. Degrons were selected from the literature, based on their size, as for AAV gene therapy application degrons smaller than 75 amino acids would be preferred to minimize the size of the resulting transgene. In some instances, degrons were designed by combining known N, or C-terminal degrons.

Degrons were cloned at the C-terminus of the N-fragment and at the N-terminus of the C-fragment, both fragments intended to reconstitute the full length EGFP protein as a representative example of a soluble cytosolic protein. In FIG. 9 a summary of the results can be observed in which it can be seen that inclusion of one, or two degrons (one on each EGFP fragment) eliminated undesired the starting material fragments without negatively affecting the splicing yields. A similar result was observed with a different degron (FIG. 14) illustrating the generality of the approach.

Degrons were cloned at the C-terminus of the N-fragment and at the N-terminus of the C-fragment, both fragments intended to reconstitute the full length ABCA4 protein at different split positions, including 1140, 1150 and 1179, as representative examples. Table 6 below shows all the degrons tested, including size, sequence, their origin and also the ligase responsible for their ubiquitination and eventual degradation.

TABLE 6

| | | | | Human |
|---|---|---|---|---|
| | | | | ortho- |
| Name | bp | | DD origin | log |
| DD1 | 39 | RISFGSPPPMAGG | H. sampiens | KLHDC2 |
| L2 | 105 | SLISLPLPTRVKFSSLLLIRI | S. cerevisiae | UBE2G2 |
| | | MKIITMTFPKKLRS | | |
| L9 | 139 | SNPFSSLFGASLLIDSVSLKS | S. cerevisiae | UBE2G2 |
| | | NWDTSSSSCLISFFSSVMFSS | | |
| | | TTRS | | |
| SopE50 | 105 | QETTLLKEKSTEKNSLAKSIL | S. typhimurium | UBE2D3 |
| | | AVKNHFIELRSKLS | | |
| SopE70 | 189 | QETTLLKEKSTEKNSLAKSIL | S. typhimurium | UBE2D3 |
| | | AVKNHFIELRSKLSERFISHK | | |
| | | NTESSATHFHRGSASEGRAVL | | |
| PEST | 90 | RSSSPSDSDTSGFSSGSDHLS | Xenopus | KLHDC2 |
| | | DLISSLRIS | | |
| M2 | 57 | VSFAFNLNSLIVGILRFHW | S. cerevisiae | TEB4 |
| M4 | 63 | YMSILRCASGKISIAAPPYIF | S. cerevisiae | LTN1 |
| V12 | 69 | TNGSVLREFTLLELEVVTRNT | s. cerevisiae | TEB4 |
| | | EL | | |
| DD3 | 33 | TNGILKLGCQG | S. cerevisiae | TEB4 |
| DD4 | 39 | RDSFGSPPPMAGG | H. sampiens | KLHDC2 |
| DD5 | 12 | GFWI | S. cerevisiae | KLHDC2 |
| DD6 | 12 | RFKG | S. cerevisiae | TEB4 |
| DD7 | 12 | KFYK | S. cerevisiae | TEB4 |
| T1 | 63 | SVLEENRPFAQQLSNVYFTIL | S. cerevisiae | TEB4 |
| T2 | 63 | KLKGLGKRCKRREDLEIRFIL | S. cerevisiae | TEB4 |
| T3 | 63 | WKLLLWVGLVLVLKHHDGAAH | S. cerevisiae | TEB4 |

Degrons were tested to confirm if they were able to induce the degradation of the starting materials, and the excised inteins, and also to confirm their effect on the yield of the protein trans-splicing reaction, that is, their ability to reconstitute the full length ABCA4 protein. Cells were co-transfected with the N- and C-terminal constructs, containing the different degrons indicated above. Cells were lysed and analyzed by Western Blot to detect the presence of the PTS product, with and without degrons. In FIG. 18, results in which the same degron was used at the N and C-fragment are shown. The gel on the left panel (FIG. 18C) shows the PTS product (ABCA4), as well as the starting materials (ABCAN-IntN-DD and DD-IntC-ABCAC) for each reaction. PTS corresponds to the result when no-degron was used, the label in the other lanes indicates which degron was used in each case. The right-hand panel (in FIG. 18C) provides the quantification of the amount of PTS product in each case. It can be seen that, surprisingly some degrons provide higher yield than the constructs without degron. Interestingly, all degrons work, and with all of them at least 25% of the product obtained without degron can be reconstituted. Some degrons allow to reconstitute more than 50% of the levels obtained without degron, and finally, there are some degrons that yield 100% (or higher) reconstitution. In particular, degrons that yield 100% (or higher) reconstitution were the following: DD1, V12, M4, L2, L9, DD3, and SopE100.

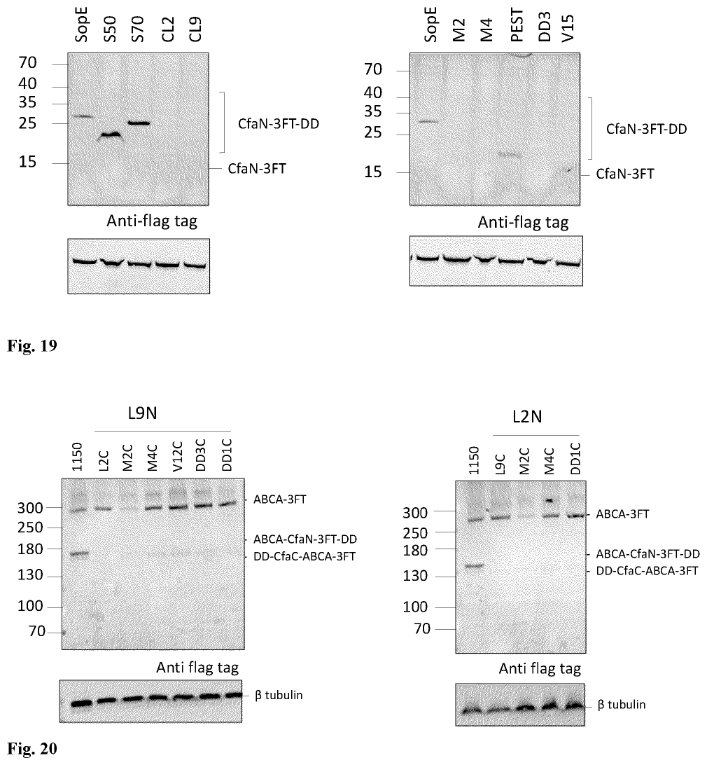

The effect of degrons on the excised intein were also studied (FIG. 19). The PTS reactions with different degrons were analysed by WB (western Blot) using a high percentage acrylamide gel to detect the inteins. As it can be seen several degrons reduce the amount of excised inteins compared to others (for example SpoE and PEST), while others, like L2 (SEQ ID NO 52), L9 (SEQ ID NO 54), M2 (SEQ ID NO 47), M4 (SEQ ID NO 35) or DD3 (SEQ ID NO 45), completely eliminate the intein band.

Additionally, we performed experiments to study the effect of using different degrons at the N- or C-terminal positions. As an example, in FIG. 20 we show a Western blot analysis wherein the degron at the N-fragment is fixed and wherein the degron at the C-fragment is changed. The results shown in FIG. 20 show that combining some of the preferred degrons a further improvement of the result can be obtained achieving a further reduction of the starting materials, without a reduction on the yield of the desired spliced product. These combinations might be useful for certain applications, particularly when completely eliminating any starting material is critical.

Based on the obtained results we could classify degrons in three categories:
Degrons that allow to reconstitute the target protein with at least 25% the yield obtained in the absence of degron.
Degrons that allow to reconstitute the target protein with yields approx. above 50% of the yield obtained in the absence of a degron.
Degrons that allow to reconstitute the target protein with yields equal or better than those obtained in the absence of a degron.

When using the combination of CfaN and CfaC or CfaCmut inteins, it was shown that several sites in ABCA4 were suitable for the use of the invention described here. Specifically, sites between the C-terminus of Nucleotide Binding Domain 1 (NBD1) and the seventh trans-membrane domain (TMD), including positions 1140, 1150, 1177, 1179 and 1188. ABCA4 reconstitution yields were best at positions 1140, 1150 and 1179, while position 1188 worked with very low efficiently, but still resulted in higher levels of ABCA4 reconstitution than those obtained using the non-consensus Npu intein.

The combination of the CfaN and CfaC mut inteins provides increased promiscuity. In fact, we showed here for the first time that this increased promiscuity is observed in a trans-membrane protein such as ABCA4, where high reconstitution yields are obtained using the CfaN-CfaCmutant pair at different splicing sites. In addition, such splicing reaction is shown to be very effective when these inteins are used (CfaN and CfaC mut inteins), as less than 10% of each of the starting materials are observed (estimated as described in FIG. 12), which was not expected based on the previously reported results using the Npu intein with ABCA4 (Auricchio et al. 2019), where a significant portion of starting material remained without reacting, suggesting that protein-trans splicing was inherently more challenging for such a membrane protein.

Based on the results reported here we conclude that any site with the following characteristics would work efficiently when the consensus intein CfaN and CfaCmutant pair are used: (1) a site outside of an enzymatic or trans-membrane domain, (2) a site such as the +1 position (as described above) of the split site should correspond to a nucleophilic residue such as Cys or Ser, and the +2 position would be any site, except Pro.

TABLE 7

| Construct | ^{a}Sequence | SEQ NO |
|---|---|---|
| ABCA4^{N}-1177 | SEQ ID NO 106 (from 1 to 1176) | 87 |
| ABCA4^{C}-1177 | SEQ ID NO 106 (from 1177 to 2273) | 88 |
| ABCA4^{N}-1179 | SEQ ID NO 106 (from 1 to 1178) | 89 |
| ABCA4^{C}-1179 | SEQ ID NO 106 from (1179 to 2273) | 90 |
| ABCA4-1177- | SEQ ID NO 87-SEQ ID NO 27 | 91 |
| Cfa^{N} | | |
| ABCA4-1177- | SEQ ID NO 29-SEQ ID NO 88 | 92 |
| Cfa^{Cmut} | | |
| ABCA4-1179- | SEQ ID NO 89-SEQ ID NO 27 | 93 |
| Cfa^{N} | | |
| ABCA4-1179- | SEQ ID NO 29-SEQ ID NO 90 | 94 |
| Cfa^{Cmut} | | |
| ABCA4^{N}-1096 | SEQ ID NO 106 (from 1 to 1095) | 95 |
| ABCA4^{C}-1096 | SEQ ID NO 106 (from 1096 to 2273) | 96 |
| ABCA4^{N}-1185 | SEQ ID NO 106 (from 1 to 1184) | 97 |
| ABCA4^{C}-1185 | SEQ ID NO 106 (from 1185 to 2273) | 98 |
| ABCA4-1096- | SEQ ID NO 95-SEQ ID NO 38 | 99 |
| gp41^{N} | | |
| ABCA4-1096- | SEQ ID NO 104-SEQ ID NO 96 | 100 |
| gp41^{C} | | |
| ABCA4-1185- | SEQ ID NO 97-SEQ ID NO 38 | 101 |
| gp41^{N} | | |
| ABCA4-1185- | SEQ ID NO 104-SEQ ID NO 98 | 102 |
| gp41^{C} | | |

### CLAUSES

1. A composition comprising a first polynucleotide encoding a polynucleotide comprising a Split intein N-fragment directly linked via a peptide bond, optionally through a peptide linker, to the N-terminal fragment of a protein to be reconstituted, and a second polynucleotide encoding a polynucleotide comprising a Split intein C-fragment directly linked via a peptide bond, optionally through a peptide linker, to the C-terminal fragment of the protein to be reconstituted;
   wherein both components of the composition may be packed together in a single formulation or separately in different formulations;
   wherein the first and the second polynucleotides, respectively, encode the N-terminal fragment and the C-terminal fragment of the protein to be reconstituted, in such a way that when both fragments are combined, the N-terminal fragment of the protein is linked to the C-terminal fragment of the protein generating the whole protein;
   wherein each polynucleotide must encode a split intein so that, once translated into a protein, the N-terminal and C-terminal sequences become separate fragments that can non-covalently re-associate, or reconstitute, into an intein that is functional for trans-splicing reactions; and wherein
   the protein to be reconstituted is of more than 25 KDa.
2. The composition of clause 2, wherein the first polynucleotide encodes a split intein N-fragment directly linked via a peptide bond to a degron, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and wherein the second polynucleotide encodes a split intein C-fragment directly linked via a peptide bond to a degron, wherein the degron is linked to the intein C-fragment via the N-terminus of the intein, with or without a linker between the intein C-fragment and the degron, and wherein the C-terminus of the Split intein C-fragment is directly linked via a peptide bond to the C-terminal fragment of the protein to be reconstituted.
3. The composition of any of clauses 1 or 2, wherein the first polynucleotide encodes the CfaN intein of SEQ ID NO 27 or any of its variants, and the second polynucleotide encodes the CfaC intein of SEQ ID NO 28, or any of its variants, wherein variants are understood as split intein N or C-fragments of SEQ ID NO: 27 or SEQ ID NO 28 having at least 90% sequence identity with any of these sequences such as CfaCmut (SEQ ID NO 29).
4. The composition of any of clauses 2 or 3, wherein degrons are selected from the list consisting of SEQ ID NO 40 to 59, 34, 35 and 37.
5. The composition of clause 2, wherein the split inteins are as defined in claim 3 and wherein the degrons are selected from the list consisting of SEQ ID NO 42, 43, 49, 50, 51, 54 and 34.
6. The composition of any of clauses 1 to 5, wherein both polynucleotides are comprised within vectors that allow the propagation of said polynucleotides in suitable host cells.
7. The composition of clause 6, wherein the are adeno associated viruses (AAV).
8. The composition of clause 7, wherein the vectors are AAVs of serotype 1, 2, 3, 4, 5, 6, 7, 8, or 9.
9. The composition of any of clauses 1 to 8, wherein the gene coding the whole protein is selected from the list consisting of any of the proteins listed in table 1.
10. The composition as defined in any of clauses 1 to 9, for use in therapy.
11. A method for expressing a gene of interest in a cell, which comprises:
(i) contacting the cell with
(a) a first polynucleotide as defined in clause 1, and
(b) a second polynucleotide as defined in clause 1,
(ii) allowing the expression of the first and the second polynucleotides so that the first and the second fusion proteins are produced and
(iii) allowing the contact between the first and second proteins so that the split intein N-fragment binds to the split intein C-fragment to form an intein intermediate and the intein intermediate reacts to covalently link the C-terminus of the first polypeptide of interest to the N-terminus of the second polypeptide of interest.
12. A method for expressing a gene of interest in a cell, which comprises:
(i) contacting the cell with
(a) a first polynucleotide as defined in clause 2, and
(b) a second polynucleotide as defined in clause 2,
(ii) allowing the expression of the first and the second polynucleotides so that the first and the second fusion proteins are produced and
(iii) allowing the contact between the first and second proteins so that the split intein N-fragment binds to the split intein C-fragment to form an intein intermediate and the intein intermediate reacts to covalently link the C-terminus of the first polypeptide of interest to the N-terminus of the second polypeptide of interest.
13. The method of any of clause 11 or 12, wherein the first polynucleotide encodes the CfaN intein of SEQ ID NO 27 or any of its variants, and the second polynucleotide encodes the CfaC intein of SEQ ID NO 28 or any of its variants, wherein variants are understood as split intein N or C-fragments of SEQ ID NO: 27 or SEQ ID NO 28 having at least 90% sequence identity with any of these sequences such as CfaCmut (SEQ ID NO 29).
14. The method of any of clauses 11 or 12, wherein the split inteins are as defined in claim 3 and wherein the degrons are selected from the list consisting of SEQ ID NO 42, 43, 49, 50, 51, 54 and 34.
15. The method of any of clauses 11 to 14, wherein both polynucleotides are comprised within adeno associated viruses (AAV).

1. A composition comprising
a. a first polynucleotide encoding a polypeptide comprising a Split intein N-fragment, wherein the Split intein N-fragment is selected from the list consisting of the CfaN of SEQ ID NO 27, the CatN of SEQ ID NO 30 and the Gp41N of SEQ ID NO 38, or any functionally equivalent variants thereof such as ConN of SEQ ID NO 39, directly linked via a peptide bond, optionally through a peptide linker, to the N-terminal fragment of a protein to be reconstituted; and
b. a second polynucleotide encoding a polypeptide comprising a Split intein C-fragment, wherein the Split intein C-fragment is selected from the list consisting of the CfaC of SEQ ID NO 28, the CatC of SEQ ID NO 31 and the Gp41C of SEQ ID NO 104, or any functionally equivalent variants thereof such as CfaCmut (SEQ ID NO 29) or ConC (SEQ ID NO 105), directly linked via a peptide bond, optionally through a peptide linker, to the C-terminal fragment of the protein to be reconstituted;
wherein both polynucleotides of the composition may be packed together in a single formulation or separately in different formulations;
wherein the first and the second polynucleotides, respectively, encode the N-terminal fragment and the C-terminal fragment of the protein to be reconstituted, in such a way that when both fragments are combined, the N-terminal fragment of the protein is linked to the C-terminal fragment of the protein generating the whole protein;
wherein the protein to be reconstituted is of more than 25 KDa; and
wherein the composition is further characterized in that:
the split intein N-fragment is further directly linked via a peptide bond to a degron, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and/or
the split intein C-fragment is further directly linked via a peptide bond to a degron, wherein the degron is linked to the intein C-fragment via the N-terminus of the intein, with or without a linker between the intein C-fragment and the degron, and wherein the C-terminus of the Split intein C-fragment is directly linked via a peptide bond to the C-terminal fragment of the protein to be reconstituted

2. The composition according to claim 1, wherein the Split intein N-fragment is the CfaN of SEQ ID NO 27, directly linked via a peptide bond, optionally through a peptide linker, to the N-terminal fragment of a protein to be reconstituted, and the Split intein C-fragment is the CfaC of SEQ ID NO 28, directly linked via a peptide bond, optionally through a peptide linker, to the C-terminal fragment of the protein to be reconstituted.

3. The composition according to claim 1, wherein the Split intein N-fragment is the CfaN of SEQ ID NO 27, directly linked via a peptide bond, optionally through a peptide linker, to the N-terminal fragment of a protein to be reconstituted, and the Split intein C-fragment is the CfaCmut of SEQ ID NO 29, directly linked via a peptide bond, optionally through a peptide linker, to the C-terminal fragment of the protein to be reconstituted.

4. The composition according to any of claims 1 to 3, wherein the degron directly linked to the split intein N-fragment and/or split intein C-fragment is selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7.

5. The composition according to any of claims 1 to 3, wherein the degron directly linked to the split intein N-fragment and/or split intein C-fragment is selected from the list consisting of DD1, DD3, PEST, SopE, L2, L9, M4, or V12.

6. The composition according to any of claims 1 to 3, wherein the degron directly linked to the split intein N-fragment and/or split intein C-fragment is selected from the list consisting of SopE, L2, L9, M4, or V12.

7. The composition according to claim 2, wherein the degron directly linked to the split intein N-fragment and/or split intein C-fragment is selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7.

8. The composition according to claim 2, wherein the degron directly linked to the split intein N-fragment and/or split intein C-fragment is selected from the list consisting of DD1, DD3, PEST, SopE, L2, L9, M4, or V12.

9. The composition according to claim 2, wherein the degron directly linked to the split intein N-fragment and/or split intein C-fragment is selected from the list consisting of SopE, L2, L9, M4, or V12.

10. The composition according to claim 3, wherein the degron directly linked to the split intein N-fragment and/or split intein C-fragment is selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7.

11. The composition according to claim 3, wherein the degron directly linked to the split intein N-fragment and/or split intein C-fragment is selected from the list consisting of DD1, DD3, PEST, SopE, L2, L9, M4, or V12.

12. The composition according to claim 3, wherein the degron directly linked to the split intein N-fragment and/or split intein C-fragment is selected from the list consisting of SopE, L2, L9, M4, or V12.

13. The composition according to any of claims 1 to 12, wherein the composition is characterized in that:
the split intein N-fragment is further directly linked via a peptide bond to a degron, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
the split intein C-fragment is further directly linked via a peptide bond to a degron, wherein the degron is linked to the intein C-fragment via the N-terminus of the intein, with or without a linker between the intein C-fragment and the degron, and wherein the C-terminus of the Split intein C-fragment is directly linked via a peptide bond to the C-terminal fragment of the protein to be reconstituted

14. The composition of claim 13, wherein
a. the first polynucleotide encoding a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaC of SEQ ID NO 28 or CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted.

15. The composition of claim 13, wherein
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and further directly linked via a peptide bond to a degron selected from the list consisting of DD1, DD3, PEST, SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaC of SEQ ID NO 28 or CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and further directly linked via a peptide bond to a degron selected from the list consisting of DD1, DD3, PEST, SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted.

16. The composition of claim 13, wherein
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and further directly linked via a peptide bond to a degron selected from the list consisting of SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaC of SEQ ID NO 28 or CfaCmut of SEQ ID NO 29, or any functionally equivalent variant thereof, directly linked via a peptide bond, optionally through a peptide linker, to the C-terminal fragment of a protein to be reconstituted, and further directly linked via a peptide bond to a degron selected from the list consisting of SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted.

17. The composition of claim 13, wherein
a. the first polynucleotide encodes a polypeptide comprising a Split intein N-fragment selected from the list consisting of Gp41N of SEQ ID NO 38 or any functionally equivalent variant thereof, directly linked via a peptide bond, optionally through a peptide linker, to the N-terminal fragment of a protein to be reconstituted, and further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of Gp41C of SEQ ID NO 104 or any functionally equivalent variant thereof, directly linked via a peptide bond, optionally through a peptide linker, to the C-terminal fragment of a protein to be reconstituted, and further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 or DD7, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted.

18. The composition of any of claims 1 to 17, wherein the first and the second polynucleotides encode the N-terminal fragment and the C-terminal fragment of the ABCA4 protein respectively, in such a way that when both polynucleotides are translated into their respective protein complexes and combined according to the methods of the invention, the N-terminal fragment of the ABCA4 protein is linked to the C-terminal fragment of the ABCA4 protein thus generating the whole ABCA4 protein.

19. The composition of claim 18, wherein
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and further directly linked via a peptide bond to a degron selected from the list consisting of CL1, Deg1, PEST, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and further directly linked via a peptide bond to a degron selected from the list consisting of CL1, Deg1, PESt, DD1, DD2, DD3, M1, M2, SopE, SopE-1-78, SopE-15-78, SopE-15-50, L2, L6, L9, L10, L11, L12, L15, L16, M3, M4, M5, V12, DD4, DD5, DD6 and DD7, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
wherein said first polynucleotide encodes positions 1-1149, 1-1139, 1-1176, or 1-1178 of the N-terminal fragment of the ABCA4 protein; and wherein the second polynucleotide encodes positions 1150-2273, 1140-2273, 1177-2273, or 1179-2273 of the C-terminal fragment of the ABCA4 protein, wherein when said first polynucleotide encodes position 1-1149 the second polynucleotide encodes positions 1150-2273, wherein when said first polynucleotide encodes position 1-1139 the second polynucleotide encodes positions 1140-2273, wherein when said first polynucleotide encodes position 1-1176 the second polynucleotide encodes positions 1177-2273, and wherein when said first polynucleotide encodes position 1-1178 the second polynucleotide encodes positions 1179-2273.

20. The composition of claim 18, wherein
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and further directly linked via a peptide bond to a degron selected from the list consisting of DD1, DD3, PEST, SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and further directly linked via a peptide bond to a degron selected from the list consisting of DD1, DD3, PEST, SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
wherein said first polynucleotide encodes positions 1-1149, 1-1139, 1-1176, or 1-1178 of the N-terminal fragment of the ABCA4 protein; and wherein the second polynucleotide encodes positions 1150-2273, 1140-2273, 1177-2273, or 1179-2273 of the C-terminal fragment of the ABCA4 protein, wherein when said first polynucleotide encodes position 1-1149 the second polynucleotide encodes positions 1150-2273, wherein when said first polynucleotide encodes position 1-1139 the second polynucleotide encodes positions 1140-2273, wherein when said first polynucleotide encodes position 1-1176 the second polynucleotide encodes positions 1177-2273, and wherein when said first polynucleotide encodes position 1-1178 the second polynucleotide encodes positions 1179-2273.

21. The composition of claim 18, wherein
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and further directly linked via a peptide bond to a degron selected from the list consisting of SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaCmut of SEQ ID NO 29 or NpuCmut of SEQ ID NO 36, or any functionally equivalent variant thereof, directly linked via a peptide bond, optionally through a peptide linker, to the C-terminal fragment of a protein to be reconstituted, and further directly linked via a peptide bond to a degron selected from the list consisting of SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
wherein said first polynucleotide encodes positions 1-1149, 1-1139, 1-1176, or 1-1178 of the N-terminal fragment of the ABCA4 protein; and wherein the second polynucleotide encodes positions 1150-2273, 1140-2273, 1177-2273, or 1179-2273 of the C-terminal fragment of the ABCA4 protein, wherein when said first polynucleotide encodes position 1-1149 the second polynucleotide encodes positions 1150-2273, wherein when said first polynucleotide encodes position 1-1139 the second polynucleotide encodes positions 1140-2273, wherein when said first polynucleotide encodes position 1-1176 the second polynucleotide encodes positions 1177-2273, and wherein when said first polynucleotide encodes position 1-1178 the second polynucleotide encodes positions 1179-2273.

22. The composition of claim 18, wherein
a. the first polynucleotide encodes a polypeptide comprising a Split intein N-fragment selected from the list consisting of CfaN of SEQ ID NO 27 or any functionally equivalent variant thereof, directly linked via a peptide bond, optionally through a peptide linker, to the N-terminal fragment of a protein to be reconstituted, and further directly linked via a peptide bond to a degron selected from the list consisting of DD1, DD3, PEST, SopE, L2, L9, M4, or V12, wherein the degron is linked to the intein N-fragment via the C-terminus of the intein, with or without a linker between the intein N-fragment and the degron, and wherein the N-terminus of the Split intein N-fragment is directly linked via a peptide bond to the N-terminal fragment of the protein to be reconstituted; and
b. the second polynucleotide encodes a polypeptide comprising a Split intein C-fragment selected from the list consisting of CfaC of SEQ ID NO 28, CfaCmut of SEQ ID NO 29 or NpuCmut of SEQ ID NO 36, or any functionally equivalent variants thereof, directly linked via a peptide bond, optionally through a peptide linker, to the C-terminal fragment of a protein to be reconstituted, and further directly linked via a peptide bond to a degron selected from the list consisting of DD1, DD3, PEST, SopE, L2, L9, M4, or V12, wherein the degron is linked to the intein C-fragment via the N-terminus of the intein, with or without a linker between the intein N-fragment and the degron, and wherein the C-terminus of the Split intein N-fragment is directly linked via a peptide bond to the N-terminal fragment of the protein to be reconstituted;
wherein said first polynucleotide encodes positions 1-1149 of the N-terminal fragment of the ABCA4 protein; and wherein the second polynucleotide encodes positions 1150-2273 of the C-terminal fragment of the ABCA4 protein, wherein when said first polynucleotide encodes position 1-1149 the second polynucleotide encodes positions 1150-2273.

23. The composition of claim 18, wherein the first polynucleotide is as defined in claim 17a and said first polynucleotide encodes positions 1-1095, or 1-1185 of the N-terminal fragment of the ABCA4 protein; and wherein the second polynucleotide is as defined in claim 17b and said second polynucleotide encodes positions 1096-2273, or 1186-2273 of the C-terminal fragment of the ABCA4 protein, wherein when said first polynucleotide encodes position 1-1095 the second polynucleotide encodes positions 1096-2273, and wherein when said first polynucleotide encodes position 1-1185 the second polynucleotide encodes positions 1186-2273.

24. The composition of any of claims 1 to 23, wherein both polynucleotides are comprised within vectors that allow the propagation or insertion of said polynucleotides in suitable host cells.

25. The composition of claim 24, wherein the vectors are adeno associated viruses (AAV).

26. The composition of claim 25, wherein the vectors are AAVs of serotype 1, 2, 3, 4, 5, 6, 7, 8, or 9.

27. The composition as defined in any of claims 1 to 26, for use in therapy.

28. A method for expressing a gene of interest in a cell, which comprises:
(i) contacting the cell with
a first polynucleotide and a second polynucleotide as defined in any of claims 1 to 12, wherein at least one of the said polynucleotides encodes a split intein fragment directly linked via a peptide bond to a degron,
(ii) allowing the expression of the first and the second polynucleotides so that the first and the second fusion proteins are produced and
(iii) allowing the contact between the first and second proteins so that the split intein N-fragment binds to the split intein C-fragment to form an intein intermediate and the intein intermediate reacts to covalently link the C-terminus of the first polypeptide of interest to the N-terminus of the second polypeptide of interest.

29. A method for expressing a gene of interest in a cell, which comprises:
(i) contacting the cell with
a first polynucleotide and a second polynucleotide as defined in any of claims 13 to 23 wherein both of the said polynucleotides encode a split intein fragment directly linked via a peptide bond to a degron, and
(ii) allowing the expression of the first and the second polynucleotides so that the first and the second fusion proteins are produced and
(iii) allowing the contact between the first and second proteins so that the split intein N-fragment binds to the split intein C-fragment to form an intein intermediate and the intein intermediate reacts to covalently link the C-terminus of the first polypeptide of interest to the N-terminus of the second polypeptide of interest.

30. The method of any of claims 28 to 29, wherein both polynucleotides are comprised within adeno associated viruses (AAV).
